(19) United States
(12) Patent Application Publication
(10) Pub. No.: US 2026/0109684 A1
(43) Pub. Date: Apr. 23, 2026

(54) QUINOLINE COMPOUNDS AS MODULATORS OF RAGE ACTIVITY AND USES THEREOF

(71) Applicants: NEW YORK UNIVERSITY, New York, NY (US); THE RESEARCH FOUNDATION FOR THE STATE UNIVERSITY OF NEW YORK, Albany, NY (US)

(72) Inventors: Ann Marie SCHMIDT, Franklin Lakes, NJ (US); Michaele MANIGRASSO, New York, NY (US); Ravichandran RAMASAMY, Ardsley, NY (US); Alexander SHEKHTMAN, Glenmont, NY (US); Robert DEVITA, Westfield, NJ (US)

(73) Assignees: NEW YORK UNIVERSITY, New York, NY (US); THE RESEARCH FOUNDATION FOR THE STATE UNIVERSITY OF NEW YORK, Albany, NY (US)

(21) Appl. No.: 19/116,027

(22) PCT Filed: Sep. 27, 2023
(86) PCT No.: PCT/US2023/033831

§ 371 Date: Mar. 27, 2025

Related U.S. Application Data
(60) Provisional application No. 63/410,824, filed on Sep. 28, 2022.

(51) Int. Cl. C07D 401/10; A61K 31/5377; A61P 17/02; C07D 215/48; C07D 401/04

(52) U.S. Cl. CPC C07D 401/10; A61K 31/5377; A61P 17/02; C07D 215/48; C07D 401/04

(57) ABSTRACT

Quinoline compounds are disclosed that have a formula represented by the following; and wherein Cy, R1, R4a, R4b, and n are as described herein. The compounds may be prepared as compositions, e g., pharmaceutical compositions, or as dosage forms, e.g., pharmaceutical dosage forms, and may be used for the prevention and treatment of a variety of conditions in mammals including humans, including by way of non-limiting example, diabetes complications, inflammation, and neurodegeneration, obesity, cancer, ischemia/reperfusion injury, cardiovascular disease, COVID-19 complications, and other diseases related to RAGE activity.

### CROSS REFERENCE TO RELATED APPLICATION

This patent application claims the benefit of U.S. Provisional Application No. 63/410,824, filed on Sep. 28, 2022, the disclosure of which is incorporated by reference herein in its entirety.

### GOVERNMENTAL SUPPORT

This invention was made with government support under R01 DK122456 and R01 DK103032 awarded by the National Institutes of Health. The government has certain rights in the invention.

### FIELD OF INVENTION

The present invention relates to quinoline compounds capable of modulating the receptor for advanced glycation end products (RAGE) activity. Specifically, this invention relates to quinoline compounds capable of modulating the interaction of RAGE and its ligands, and uses of such compounds to treat diseases or conditions related to RAGE activity. More particularly, the quinoline compounds may be used to treat diabetes complications, infection, inflammation, and neurodegeneration, obesity, cancer, ischemia/reperfusion injury, cardiovascular disease and other diseases related to RAGE activity. Also encompassed herein are compositions of quinoline compounds, pharmaceutical compositions of quinoline compounds, and methods for using same to modulate RAGE activity.

### BACKGROUND OF THE INVENTION

The receptor for advanced glycation end products (RAGE) is a multiligand cell surface macromolecule that plays a central role in the etiology of diabetes complications, obesity, inflammation, cancer and neurodegeneration. The cytoplasmic domain of RAGE, C terminal RAGE or ctRAGE (RAGE tail) is critical for RAGE-dependent signal transduction. As the most membrane proximal event, DIAPH1 (also known as mammalian Dial or mDial) binds to RAGE and is essential for RAGE ligand-stimulated phosphorylation of kinases and cellular properties such as cell proliferation/migration of smooth muscle cells; activation of cdc42 and rac1 in smooth muscle cells and transformed cells; and upregulation of early growth response 1 in hypoxic macrophages, as examples. ctRAGE contains an unusual a-turn that mediates the DIAPH1-RAGE interaction and is required for RAGE dependent signaling (Shekhtman et al, J. Bio. Chem., 2012, 287(7) 5133-5142).

RAGE-ligand interactions evoke central changes in cellular properties including stimulation of cellular migration and proliferation and leading to such pathological conditions as diabetes and its complications, Alzheimer's disease, inflammation and cancers. RAGE also plays a pivotal role in the atherosclerotic process (Schmidt, et al. (1999) Circ Res 84, 489-497). Thus, inhibition of the RAGE activity is desirable for treatment of these conditions.

US application publication, US2012/0088778 discloses azole derivatives as modulators of the interaction of RAGE and its ligands or RAGE activity. The azole compounds are reported to be useful for treatment of diseases including acute and chronic inflammation, the development of diabetic late complications, and others.

US application publication, US2010/0254983 discloses methods for treating obesity using antagonists of binding of RAGE ligands to RAGE. US application publication, US2010/0119512 discloses carboxamide derivatives as modulators of the interaction of RAGE and its ligands or RAGE activity. U.S. Pat. No. 7,361,678 discloses composition of 3,5-diphenyl-imidazole derivatives as modulators of the interaction of RAGE and its ligands or RAGE activity. International application publication, WO2007/089616, discloses tertiary amides as modulators of the interaction of RAGE and its ligands or RAGE activity. US application publication, US2010/0249038, discloses novel peptides as antagonists of RAGE.

International application publication, WO/2015/050984, discloses amino, amido, and heterocyclic compounds as modulators of RAGE activity. International application publication, WO2017/184547, discloses quinoline compounds as modulators of RAGE activity. International application publication, WO/2021/026185, discloses indole compounds as modulators of RAGE activity.

Many or most of the ligands disclosed in the above applications bind to the extracellular domain of RAGE.

In view of the above, a need exists for therapeutic agents, and corresponding pharmaceutical compositions and related methods of treatment that address conditions causally related to RAGE activity, and it is toward the fulfillment and satisfaction of that need, that the present invention is directed.

### SUMMARY OF THE INVENTION

Various non-limiting aspects and embodiments of the invention are described below.

In one aspect, the present invention provides a compound having a structure of Formula (I):

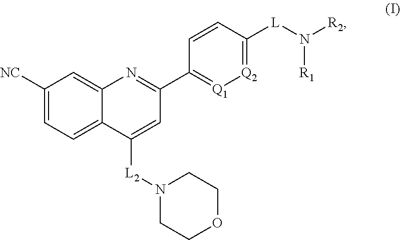

wherein:
Q₁ and Q₂ are independently ═CH— or ═N—, with a proviso that Q₁ and Q₂ are not both ═N—;
L₁ and L₂ are independently —C(═O)—, —C(═NH)—, a C₁-C₆ alkyl, or a combination thereof;
R₁ is hydrogen, a C₁-C₆ alkyl, —C(═O)—CH₃, or R₁ and Q₂ are fused to make an optionally substituted bicyclic heterocycle;
R₂ is hydrogen, a C₁-C₆ alkyl, or R₁ and R₂ are fused to make a 5- or 6-membered optionally substituted heterocycle,
or a pharmaceutically acceptable salt thereof.

In one embodiment, Q₁ is ═CH— and Q₂ is ═N—. In another embodiment, Q₁ is ═N— and Q₂ is ═CH—. In yet another embodiment, Q₁ and Q₂ are both ═CH—.

In one embodiment, L₁ is —C(═O)—. In another embodiment, L₁ is —C(═NH)—. In yet another embodiment, L₁ is a C₁-C₆ alkyl. In yet another embodiment, L₁ is a C₁-C₃ alkyl.

In one embodiment, L₁ is a —CH₂—. In another embodiment, L₁ is a —(CH₂)₂—. In yet another embodiment, L₁ is a —(C(H)CH₃)—. In yet another embodiment, L₁ is a —(CH₂)(C(H)CH₃)—.

In one embodiment, L₁ is a combination of —C(═O)— and a C₁-C₆ alkyl. In another embodiment, L₁ is a combination of —C(═O)— and a C₁-C₃ alkyl. In yet another embodiment, L₁ is —(CH₂)—C(═O)—.

In one embodiment, L₂ is —C(═O)—. In another embodiment, L₂ is a C₁-C₆ alkyl. In yet another embodiment, L₂ is a —CH₂—.

In one embodiment, R₁ is hydrogen. In another embodiment, R₁ is a C₁-C₆ alkyl. In yet another embodiment, R₁ is a C₁-C₃ alkyl. In yet another embodiment, R₁ is —CH₃. In yet another embodiment, R₁ is —C(═O)—CH₃.

In one embodiment, R₂ is hydrogen. In another embodiment, R₂ is a C₁-C₆ alkyl. In yet another embodiment, R₂ is a C₁-C₃ alkyl. In yet another embodiment, R₂ is —CH₃.

In one embodiment, R₁ and R₂ are fused to make a 5- or 6-membered optionally substituted heterocycle.

In one embodiment, R₁ and L₁ are fused to make a 5- or 6-membered optionally substituted heterocycle.

In one embodiment, R₁ and L₁ are

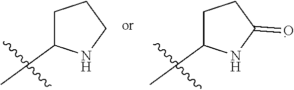 [SMILES: *C1CCCN1 ; *C1CCC(=O)N1]

In one embodiment, R₁ and Q₂ are fused to make an optionally substituted bicyclic heterocycle.

In one embodiment, the compound of the invention has the structure of Formula (II):

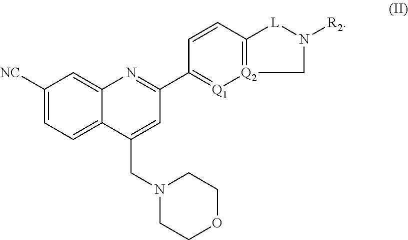

In one embodiment, the compound of the invention has the structure selected from the group consisting of:

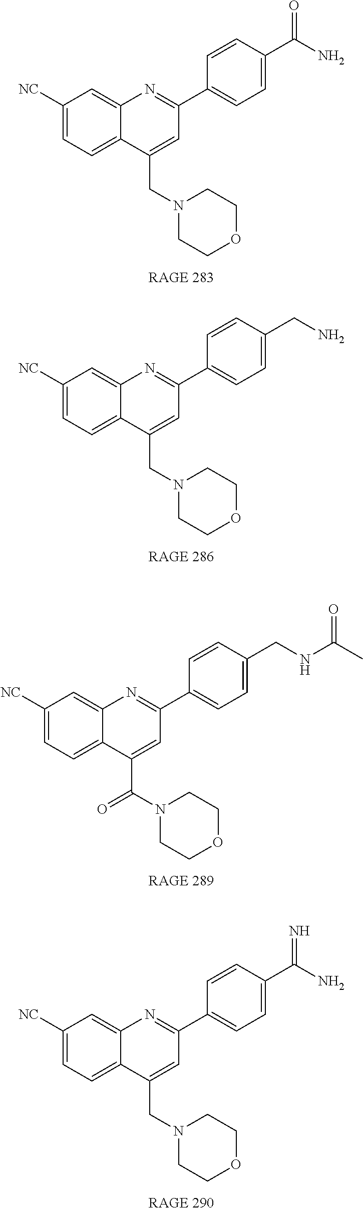 [SMILES: N#Cc1ccc2c(CN3CCOCC3)cc(-c3ccc(C(N)=O)cc3)nc2c1 ; N#Cc1ccc2c(CN3CCOCC3)cc(-c3ccc(CN)cc3)nc2c1 ; CC(=O)NCc1ccc(-c2cc(C(=O)N3CCOCC3)c3ccc(C#N)cc3n2)cc1 ; N#Cc1ccc2c(CN3CCOCC3)cc(-c3ccc(C(=N)N)cc3)nc2c1]

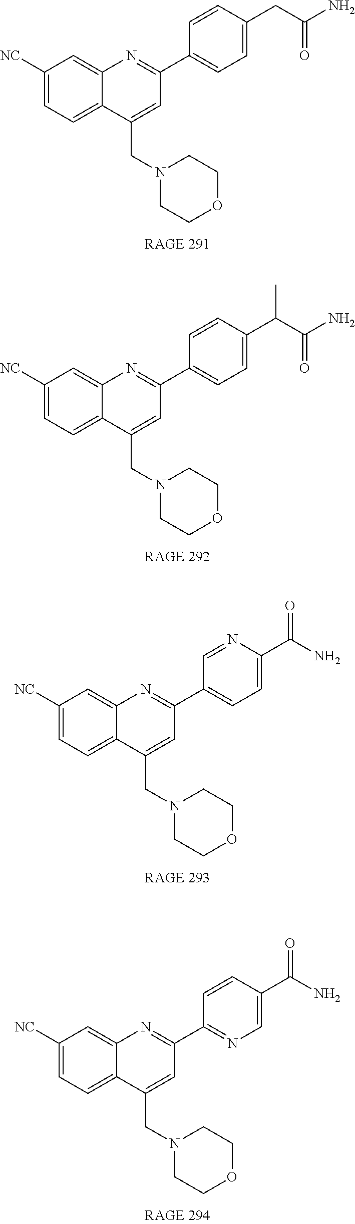 [SMILES: N#Cc1ccc2c(CN3CCOCC3)cc(-c3ccc(CC(N)=O)cc3)nc2c1 ; CC(C(N)=O)c1ccc(-c2cc(CN3CCOCC3)c3ccc(C#N)cc3n2)cc1 ; N#Cc1ccc2c(CN3CCOCC3)cc(-c3ccc(C(N)=O)nc3)nc2c1 ; N#Cc1ccc2c(CN3CCOCC3)cc(-c3ccc(C(N)=O)cn3)nc2c1]

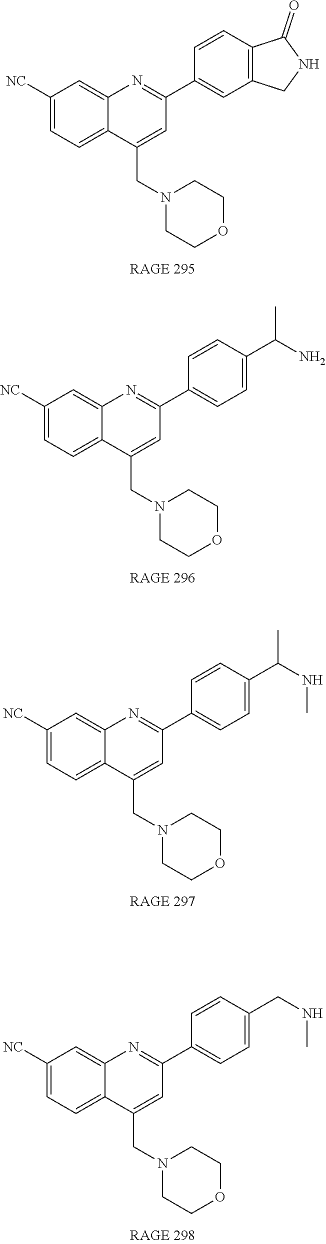 [SMILES: N#Cc1ccc2c(CN3CCOCC3)cc(-c3ccc4c(c3)CNC4=O)nc2c1 ; CC(N)c1ccc(-c2cc(CN3CCOCC3)c3ccc(C#N)cc3n2)cc1 ; CNC(C)c1ccc(-c2cc(CN3CCOCC3)c3ccc(C#N)cc3n2)cc1 ; CNCc1ccc(-c2cc(CN3CCOCC3)c3ccc(C#N)cc3n2)cc1]

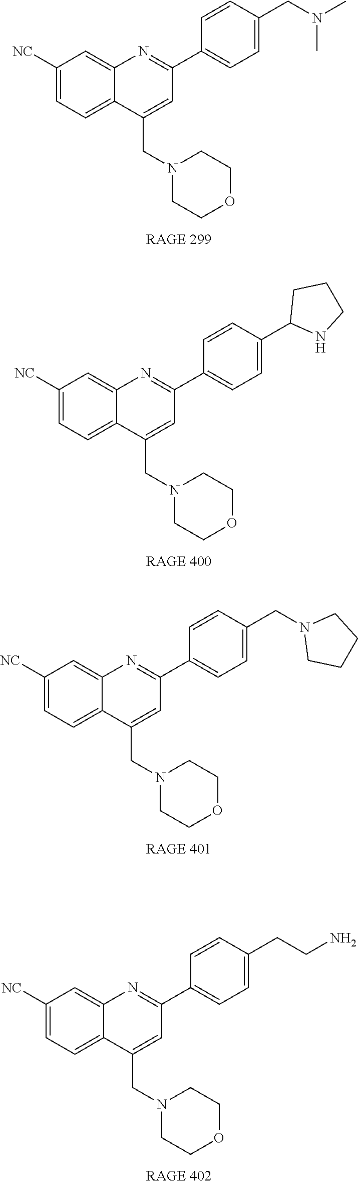 [SMILES: CN(C)Cc1ccc(-c2cc(CN3CCOCC3)c3ccc(C#N)cc3n2)cc1 ; N#Cc1ccc2c(CN3CCOCC3)cc(-c3ccc(C4CCCN4)cc3)nc2c1 ; N#Cc1ccc2c(CN3CCOCC3)cc(-c3ccc(CN4CCCC4)cc3)nc2c1 ; N#Cc1ccc2c(CN3CCOCC3)cc(-c3ccc(CCN)cc3)nc2c1]

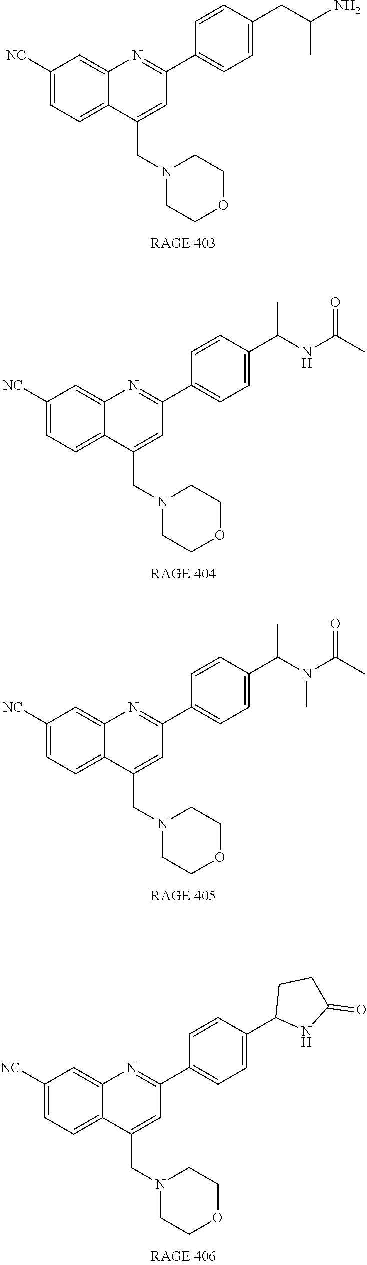 [SMILES: CC(N)Cc1ccc(-c2cc(CN3CCOCC3)c3ccc(C#N)cc3n2)cc1 ; CC(=O)NC(C)c1ccc(-c2cc(CN3CCOCC3)c3ccc(C#N)cc3n2)cc1 ; CC(=O)N(C)C(C)c1ccc(-c2cc(CN3CCOCC3)c3ccc(C#N)cc3n2)cc1 ; N#Cc1ccc2c(CN3CCOCC3)cc(-c3ccc(C4CCC(=O)N4)cc3)nc2c1]

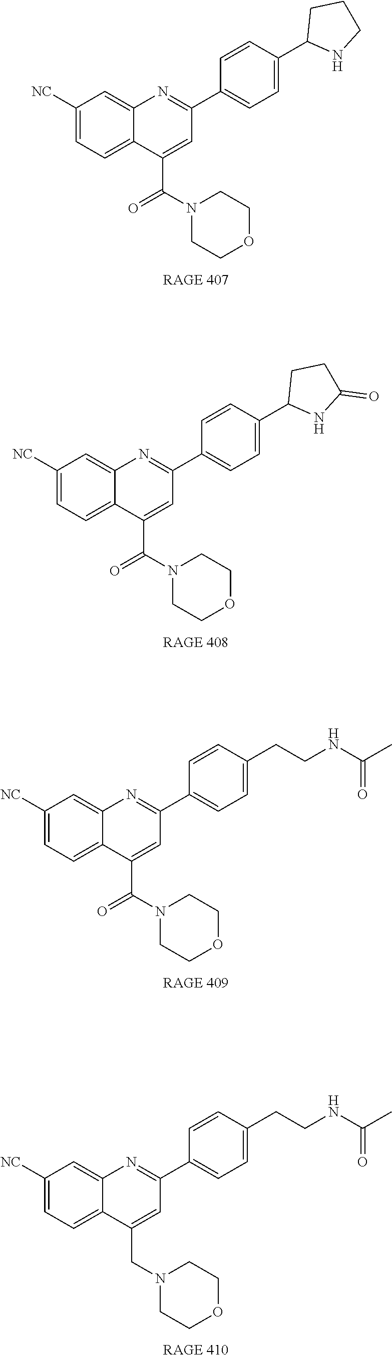 [SMILES: N#Cc1ccc2c(C(=O)N3CCOCC3)cc(-c3ccc(C4CCCN4)cc3)nc2c1 ; N#Cc1ccc2c(C(=O)N3CCOCC3)cc(-c3ccc(C4CCC(=O)N4)cc3)nc2c1 ; CC(=O)NCCc1ccc(-c2cc(C(=O)N3CCOCC3)c3ccc(C#N)cc3n2)cc1 ; CC(=O)NCCc1ccc(-c2cc(CN3CCOCC3)c3ccc(C#N)cc3n2)cc1]

or a pharmaceutically acceptable salt thereof.

In one embodiment, the compound of the invention has the structure:

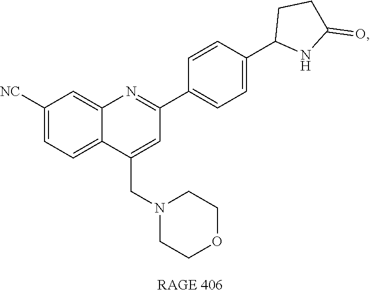 [SMILES: N#Cc1ccc2c(CN3CCOCC3)cc(-c3ccc(C4CCC(=O)N4)cc3)nc2c1]

or a pharmaceutically acceptable salt thereof.

In one embodiment, the compound is a racemic mixture of RAGE 406R and RAGE406S:

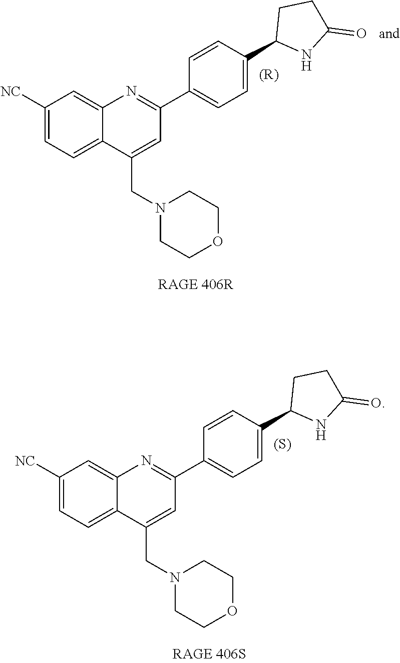

In one embodiment, the compound is RAGE 406R.

In another aspect, the present invention provides a pharmaceutical composition comprising the compound of any of the above embodiments, or a pharmaceutically acceptable salt thereof, and a pharmaceutically acceptable carrier.

In another aspect, the present invention provides a pharmaceutical dosage form comprising the compound of any of the above embodiments, or a pharmaceutically acceptable salt thereof, or the pharmaceutical composition of any of the above embodiments.

In yet another aspect, the present invention provides a method of modulating the activity of the receptor for advanced glycation end products (RAGE) in a cell comprising contacting the cell with an effective amount of the compound of any of the above embodiments, or the pharmaceutically acceptable salt thereof or a pharmaceutical composition of any of the above embodiments.

In one embodiment, modulating the activity of RAGE comprises inhibiting the activity of RAGE.

In yet another aspect, the present invention provides a method of treating or ameliorating a disease or condition associated with RAGE activity in a subject in need thereof comprising administering to the subject a therapeutically effective amount of the compound of any of the above embodiments or the pharmaceutically acceptable salt thereof or a pharmaceutical composition of any of the above embodiments.

In one embodiment, the disease or condition is selected from diabetes and its complications, insulin resistance, atherosclerosis, peripheral vascular disease and associated complications, cardiovascular disease nephropathy, retinopathy, cardio- and cerebrovascular ischemia/reperfusion injury, heart attack, myocardial infarction, ischemic cardiomyopathy, cancer, tumor invasion and metastases, acute and chronic inflammation, autoimmune diseases, neurodegeneration, arthritis, allergy asthma, obesity, pollution-associated tissue or organ damage, infection and its complications, sepsis, pneumonia, liver injury/damage, amyloidoses, skin disorders, colitis, lupus, and impaired wound healing.

In one embodiment, the disease or condition is diabetes and its complications. In another embodiment, the disease or condition is COVID-19 infection and its complications.

In yet another aspect, the present invention provides a method of treating or ameliorating diabetes and its complications in a subject in need thereof comprising administering to the subject a therapeutically effective amount of the compound of any of the above embodiments, or the pharmaceutically acceptable salt thereof or a pharmaceutical composition of any of the above embodiments.

In one embodiment, the subject is a mammal.

In one embodiment, the compound of any of the above embodiments, or the pharmaceutically acceptable salt thereof or a pharmaceutical composition of any of the above embodiments is administered orally, rectally, intraocularly, transdermally, subcutaneously, intravenously, intramuscularly, intraperitoneally, interdermally, directly into cerebrospinal fluid, intratracheally, or intranasally.

These and other aspects of the present invention will become apparent to those skilled in the art after a reading of the following detailed description of the invention, including the appended claims.

### BRIEF DESCRIPTION OF THE DRAWINGS

FIG. 1 illustrates the in vivo delayed type hypersensitivity reaction for RAGE 229, RAGE 406R, and RAGE 406S in male mice.

FIG. 2 illustrates the in vivo delayed type hypersensitivity reaction for RAGE 229, RAGE 406R, and RAGE 406S in female mice.

FIG. 3 illustrates the results of a fluorescence binding titration assay of RAGE 283 to ctRAGE.

FIG. 4 illustrates the results of a fluorescence binding titration assay of RAGE 286 to ctRAGE.

FIG. 5 illustrates the results of a fluorescence binding titration assay of RAGE 289 to ctRAGE.

FIG. 6 illustrates the results of a fluorescence binding titration assay of RAGE 290 to ctRAGE.

FIG. 7 illustrates the results of a fluorescence binding titration assay of RAGE 291 to ctRAGE.

FIG. 8 illustrates the results of a fluorescence binding titration assay of RAGE 299 to ctRAGE.

FIG. 9 illustrates the results of a fluorescence binding titration assay of RAGE 402 to ctRAGE.

FIG. 10 illustrates the results of a fluorescence binding titration assay of RAGE 406 to ctRAGE.

FIG. 11 illustrates the results of a fluorescence binding titration assay of RAGE 407 to ctRAGE.

FIG. 12 illustrates the results of a murine SMC inhibition of migration assay with RAGE 283.

FIG. 13 illustrates the results of a murine SMC inhibition of migration assay with RAGE 286.

FIG. 14 illustrates the results of a murine SMC inhibition of migration assay with RAGE 289.

FIG. 15 illustrates the results of a murine SMC inhibition of migration assay with RAGE 290.

FIG. 16 illustrates the results of a murine SMC inhibition of migration assay with RAGE 299.

FIG. 17 illustrates the results of a murine SMC inhibition of migration assay with RAGE 401.

FIG. 18 illustrates the results of a murine SMC inhibition of migration assay with RAGE 402.

FIG. 19 illustrates the results of a murine SMC inhibition of migration assay with RAGE 406.

FIG. 20 illustrates the results of a murine SMC inhibition of migration assay with RAGE 406R.

FIG. 21 illustrates the results of a murine SMC inhibition of migration assay with RAGE 406S.

FIG. 22 illustrates the results of a murine SMC inhibition of migration assay with RAGE 407.

FIGS. 23A-D illustrate the results of an in vivo test with RAGE406r in male (FIGS. 23A and 23C) and female (FIGS. 23B and 23D) ob ob mice. *p<0.05.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

RAGE and its ligands have been positioned at the center of chronic inflammation, and it is understood that chronic inflammation contributes significantly to the pathogenesis of diverse disorders. The compounds described herein are envisioned as useful for the treatment of diseases wherein inflammation plays a pathological role and RAGE contributes thereto. Such diseases and disorders include inflammatory bowel disease, delayed-type hypersensitivity, atherosclerosis, the complications of diabetes (including neuropathy and atherosclerosis), asthma, myocardial ischemia, atherosclerosis aneurysm formation, doxorubicin toxicity, acetaminophen toxicity, neurodegeneration, hyperlipidemia, preeclampsia, rheumatoid arthritis, pulmonary fibrosis, COVID-19 complications and Alzheimer's Disease. See, for example, Hofmann et al. (1999, Cell 97:889-901); Akirav et al. (2014, PLOS One9: e95678); Johnson et al. (2014, EJNMMI Res 4:26); Tekabe et al. (2014, Int J Mol Imaging 2014:695391); Song et al. (2014, Diabetes 63:1948-1965); Ullah et al. (2014, J Allergy Clin Immunol 134:440-450); Juranek et al. (2013, Brain Behav 3:701-709); Daffu et al. (2013, Int J Mol Sci 14:19891-19910); Manigrasso et al. (2014, Trends Endocrinol Metab 25:15-22); Tekabe et al. (2013, EJNMMi Res 3:37); Rai et al. (2012, J Exp Med 209:2339-2350); Ramasamy et al. (2012, Vascular Pharmacol 57:160-167); and Arumugam et al. (2012, Clin Cane Res 18:4356-4364); the entire content of each of which is incorporated herein by reference.

Further to the above, identification of inhibitors of the interaction of the RAGE cytoplasmic domain with the FHl domain of DIAPHl holds great promise for the suppression of RAGE signal transduction. Extensive experimental evidence affirms that RAGE ligands mediate their pathogenic effects via RAGE through the induction of intracellular signaling pathways. Hence, inhibitors that block RAGE-DIAPHl interaction, clearly shown to block intracellular signaling and inflammatory/cell stimulatory gene expression changes, are envisioned as therapeutic agents capable of inhibiting the effects of RAGE ligands in chronic diseases in which the ligands of RAGE accumulate. The small molecule inhibitors described herein are, therefore, set forth as novel therapeutic agents for the treatment of RAGE-associated diseases.

### Definitions

Unless defined otherwise, all technical and scientific terms used herein have the same meaning as commonly understood by one of ordinary skill in the art to which this invention belongs.

When describing the compounds, pharmaceutical compositions containing such compounds and methods of using such compounds and compositions, the following terms have the following meanings unless otherwise indicated. It should also be understood that any of the moieties defined forth below may be substituted with a variety of substituents, and that the respective definitions are intended to include such substituted moieties within their scope. It should be further understood that the terms “groups” and “radicals” can be considered interchangeable when used herein.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a mammal, e.g., a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon, bicyclic hydrocarbon, or tricyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-30 aliphatic carbon atoms. In some embodiments, aliphatic groups contain 1-20 aliphatic carbon atoms. In other embodiments, aliphatic groups contain 1-10 aliphatic carbon atoms. In still other embodiments, aliphatic groups contain 1-6 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “cycloaliphatic,” as used herein, refers to saturated or partially unsaturated cyclic aliphatic monocyclic, bicyclic, or polycyclic ring systems, as described herein, having from 3 to 14 members, wherein the aliphatic ring system is optionally substituted as defined above and described herein. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3-6 carbons. The terms “cycloaliphatic,” may also include aliphatic rings that are fused to one or more aromatic or nonaromatic rings, such as decahydronaphthyl or tetrahydronaphthyl, where the radical or point of attachment is on the aliphatic ring. In some embodiments, a carbocyclic group is bicyclic. In some embodiments, a ‘carbocyclic group is tricyclic. In some embodiments, a carbocyclic group is polycyclic. In some embodiments, “cycloaliphatic” (or “carbocycle” or “cycloalkyl”) refers to a monocyclic C₃-C₆ hydrocarbon, or a C₈-C₁₀ bicyclic hydrocarbon that is completely saturated or that contains one or more units of unsaturation, but which is not aromatic, that has a single point of attachment to the rest of the molecule, or a C₉-C₁₆ tricyclic hydrocarbon that is completely saturated or that contains one or more units of unsaturation, but which is not aromatic, that has a single point of attachment to the rest of the molecule.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 1-20 carbon atoms in its backbone (e.g., C₁-C₂₀ for straight chain, C₂-C₂₀ for branched chain), and alternatively, about 1-10 carbon atoms, or about 1 to 6 carbon atoms. In some embodiments, a cycloalkyl ring has from about 3-10 carbon atoms in their ring structure where such rings are monocyclic or bicyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

As used herein, the term “alkenyl” refers to an alkyl group, as defined herein, having one or more double bonds.

As used herein, the term “alkynyl” refers to an alkyl group, as defined herein, having one or more triple bonds.

The term “heteroalkyl” is given its ordinary meaning in the art and refers to alkyl groups as described herein in which one or more carbon atoms is replaced with a heteroatom (e.g., oxygen, nitrogen, sulfur, and the like). Examples of heteroalkyl groups include, but are not limited to, alkoxy, poly(ethylene glycol)-, alkyl-substituted amino, tetrahydrofuranyl, piperidinyl, morpholinyl, etc.

The term “aryl” used alone or as part of a larger moiety as in “aralkyl,” “aralkoxy,” or “aryloxyalkyl.” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone of as part of a larger moiety, e.g., “heteroaralkyl,” or “heteroaralkoxy,” refer to groups having 5 to 10 ring atoms (i.e., monocyclic or bicyclic), in some embodiments 5, 6, 9, or 10 ring atoms. In some embodiments, such rings have 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quatemized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be monocyclic, bicyclic, tricyclic, tetracyclic, and/or otherwise polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen.

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic, tricyclic, tetracyclic, and/or otherwise polycyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring.

The term “unsaturated,” as used herein, means that a moiety has one or more units of unsaturation.

The term “halogen” means F, Cl, Br, or I; the term “halide” refers to a halogen radical or substituent, namely —F, —Cl, —Br, or —I.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention.

Unless otherwise stated, all tautomeric forms of the compounds of the invention are within the scope of the invention.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this invention.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Unless otherwise stated, all crystalline forms of the compounds of the invention and salts thereof are also within the scope of the invention. The compounds of the invention may be isolated in various amorphous and crystalline forms, including without limitation forms which are anhydrous, hydrated, non-solvated, or solvated. Example hydrates include hemihydrates, monohydrates, dihydrates, and the like. In some embodiments, the compounds of the invention are anhydrous and non-solvated. By “anhydrous” is meant that the crystalline form of the compound contains essentially no bound water in the crystal lattice structure, i.e., the compound does not form a crystalline hydrate.

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. The different crystalline lattices can be identified by solid state characterization methods such as by X-ray powder diffraction (PXRD). Other characterization methods such as differential scanning calorimetry (DSC), thermogravimetric analysis (TGA), dynamic vapor sorption (DVS), solid state NMR, and the like further help identify the crystalline form as well as help determine stability and solvent/water content.

Crystalline forms of a substance include both solvated (e.g., hydrated) and non-solvated (e.g., anhydrous) forms. A hydrated form is a crystalline form that includes water in the crystalline lattice. Hydrated forms can be stoichiometric hydrates, where the water is present in the lattice in a certain water/molecule ratio such as for hemihydrates, monohydrates, dihydrates, etc. Hydrated forms can also be non-stoichiometric, where the water content is variable and dependent on external conditions such as humidity.

In some embodiments, the compounds of the invention are substantially isolated. By “substantially isolated” is meant that a particular compound is at least partially isolated from impurities. For example, in some embodiments a compound of the invention comprises less than about 50%, less than about 40%, less than about 30%, less than about 20%, less than about 15%, less than about 10%, less than about 5%, less than about 2.5%, less than about 1%, or less than about 0.5% of impurities. Impurities generally include anything that is not the substantially isolated compound including, for example, other crystalline forms and other substances.

### The Compounds

The present invention provides quinoline compounds capable of modulating receptor for advanced glycation end products (RAGE) activity.

Specifically, the invention provides quinoline compounds capable of modulating the interaction of RAGE and its ligands, and uses of such compounds to treat diseases or conditions related to RAGE activity.

More specifically, the invention provides quinoline compounds capable of modulating the interaction of RAGE and its ligands binding to the intracellular domain of the RAGE, and uses of such compounds to treat diseases or conditions related to RAGE activity.

In one aspect, the present invention provides a method for preventing, treating or ameliorating in a mammal a disease or condition that is causally related to RAGE activity in vivo, which comprises administering to the mammal an effective disease-treating or condition-treating amount of a compound according to Formula (I):

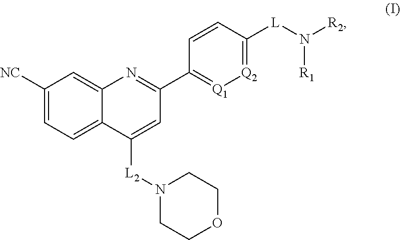

wherein:
Q₁ and Q₂ are independently —CH— or ═N—, with a proviso that Q₁ and Q₂ are not both ═N—;
L₁ and L₂ are independently —C(═O)—, —C(═NH)—, a C₁-C₆ alkyl, or a combination thereof;
R₁ is hydrogen, a C₁-C₆ alkyl, —C(═O)—CH₃, or R₁ and Q₂ are fused to make an optionally substituted bicyclic heterocycle;
R₂ is hydrogen, a C₁-C₆ alkyl, or R₁ and R₂ are fused to make a 5- or 6-membered optionally substituted heterocycle,
or a pharmaceutically acceptable salt thereof.

In one embodiment of the compound of Formula (I), Q₁ is ═CH— and Q₂ is ═N—. In another embodiment, Q₁ is ═N— and Q₂ is ═CH—. In yet another embodiment, Q₁ and Q₂ are both ═CH—.

In one embodiment of the compound of Formula (I), L₁ is —C(═O)—. In another embodiment, L₁ is —C(═NH)—.

In one embodiment, L₁ is a C₁-C₆ alkyl. In another embodiment, L₁ is a C₁-C₃ alkyl. In another embodiment, L₁ is a —CH₂—. In another embodiment, L₁ is a —(CH₂)₂—. In another embodiment, L₁ is a —(C(H)CH₃)—. In another embodiment, L₁ is a —(CH₂)(C(H)CH₃)—.

In one embodiment, L₁ is a combination of —C(═O)— and a C₁-C₆ alkyl. In another embodiment, L₁ is a combination of —C(═O)— and a C₁-C₃ alkyl. In another embodiment, L₁ is —(CH₂)—C(═O)—.

In one embodiment of the compound of Formula (I), L₂ is —C(═O)—. In another embodiment, L₂ is a C₁-C₆ alkyl. In another embodiment, L₂ is a —CH₂—.

In one embodiment of the compound of Formula (I), R₁ is hydrogen.

In another embodiment, R₁ is a C₁-C₆ alkyl. In another embodiment, R₁ is a C₁-C₃ alkyl.

In another embodiment, R₁ is —CH₃.

In another embodiment, R₁ is —C(═O)—CH₃.

In one embodiment of the compound of Formula (I), R₂ is hydrogen.

In one embodiment of the compound of Formula (I), R₂ is a C₁-C₆ alkyl. In another embodiment, R₂ is a C₁-C₃ alkyl. In another embodiment, R₂ is —CH₃.

In one embodiment of the compound of Formula (I), R₁ and R₂ are fused to make a 5- or 6-membered optionally substituted heterocycle.

In one embodiment of the compound of Formula (I), R₁ and L₁ are fused to make a 5- or 6-membered optionally substituted heterocycle. In one embodiment, R₁ and L₁ are

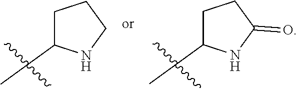 [SMILES: *C1CCCN1 ; *C1CCC(=O)N1]

In one embodiment of the compound of Formula (I), R₁ and Q₂ are fused to make an optionally substituted bicyclic heterocycle.

In one embodiment of the compound of Formula (I), the compound has the structure of Formula (II):

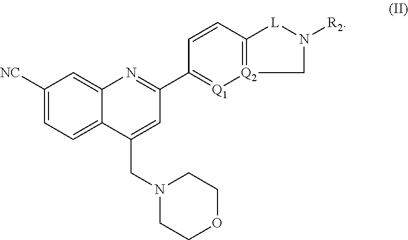

In one embodiment of the compound of Formula (I), the compound has the structure selected from the group consisting of:

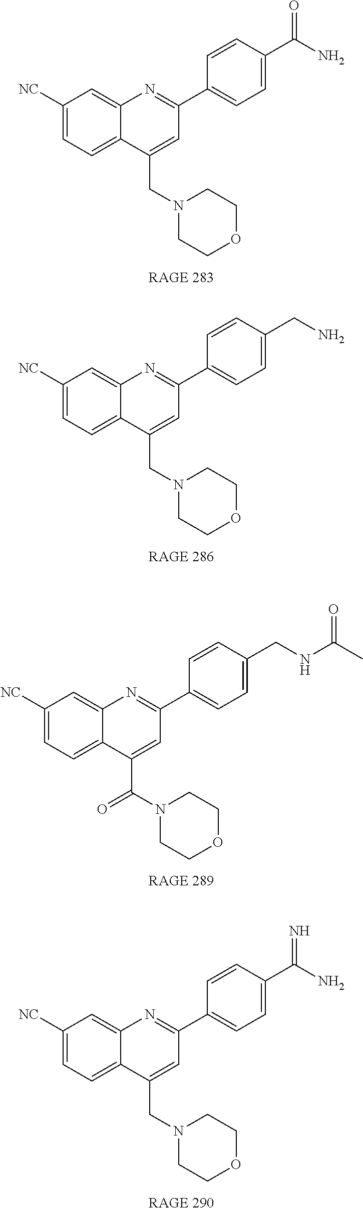 [SMILES: N#Cc1ccc2c(CN3CCOCC3)cc(-c3ccc(C(N)=O)cc3)nc2c1 ; N#Cc1ccc2c(CN3CCOCC3)cc(-c3ccc(CN)cc3)nc2c1 ; CC(=O)NCc1ccc(-c2cc(C(=O)N3CCOCC3)c3ccc(C#N)cc3n2)cc1 ; N#Cc1ccc2c(CN3CCOCC3)cc(-c3ccc(C(=N)N)cc3)nc2c1]

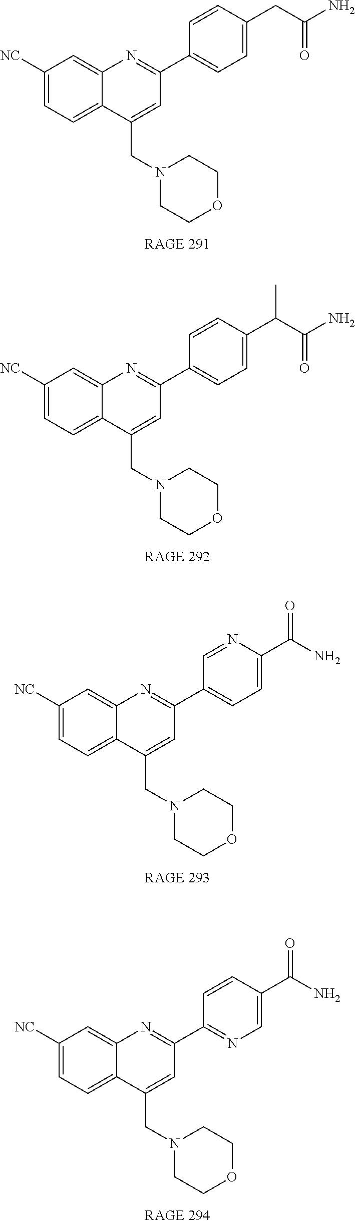 [SMILES: N#Cc1ccc2c(CN3CCOCC3)cc(-c3ccc(CC(N)=O)cc3)nc2c1 ; CC(C(N)=O)c1ccc(-c2cc(CN3CCOCC3)c3ccc(C#N)cc3n2)cc1 ; N#Cc1ccc2c(CN3CCOCC3)cc(-c3ccc(C(N)=O)nc3)nc2c1 ; N#Cc1ccc2c(CN3CCOCC3)cc(-c3ccc(C(N)=O)cn3)nc2c1]

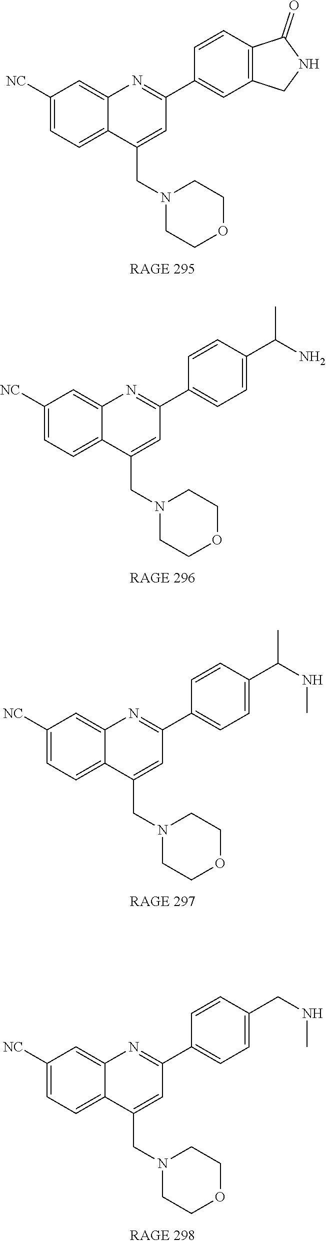 [SMILES: N#Cc1ccc2c(CN3CCOCC3)cc(-c3ccc4c(c3)CNC4=O)nc2c1 ; CC(N)c1ccc(-c2cc(CN3CCOCC3)c3ccc(C#N)cc3n2)cc1 ; CNC(C)c1ccc(-c2cc(CN3CCOCC3)c3ccc(C#N)cc3n2)cc1 ; CNCc1ccc(-c2cc(CN3CCOCC3)c3ccc(C#N)cc3n2)cc1]

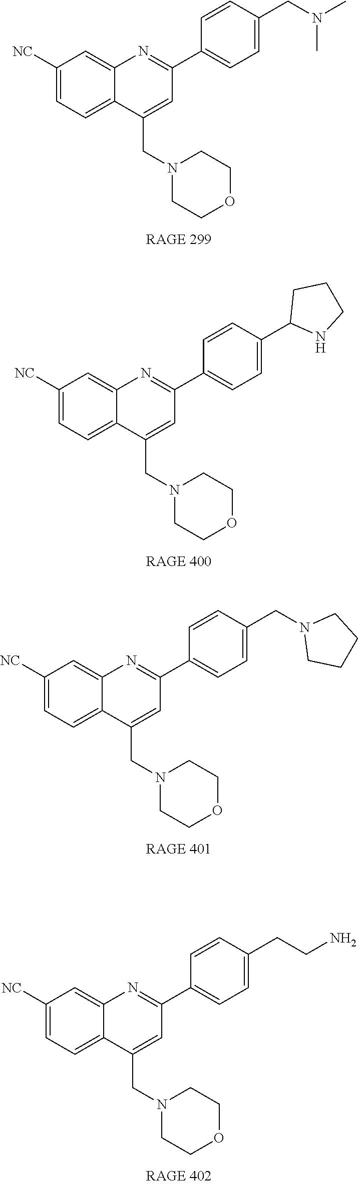 [SMILES: CN(C)Cc1ccc(-c2cc(CN3CCOCC3)c3ccc(C#N)cc3n2)cc1 ; N#Cc1ccc2c(CN3CCOCC3)cc(-c3ccc(C4CCCN4)cc3)nc2c1 ; N#Cc1ccc2c(CN3CCOCC3)cc(-c3ccc(CN4CCCC4)cc3)nc2c1 ; N#Cc1ccc2c(CN3CCOCC3)cc(-c3ccc(CCN)cc3)nc2c1]

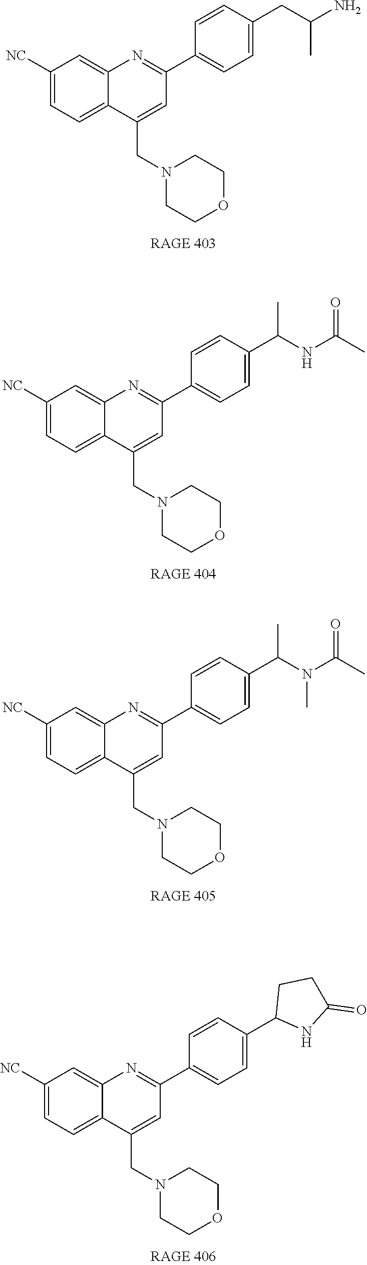 [SMILES: CC(N)Cc1ccc(-c2cc(CN3CCOCC3)c3ccc(C#N)cc3n2)cc1 ; CC(=O)NC(C)c1ccc(-c2cc(CN3CCOCC3)c3ccc(C#N)cc3n2)cc1 ; CC(=O)N(C)C(C)c1ccc(-c2cc(CN3CCOCC3)c3ccc(C#N)cc3n2)cc1 ; N#Cc1ccc2c(CN3CCOCC3)cc(-c3ccc(C4CCC(=O)N4)cc3)nc2c1]

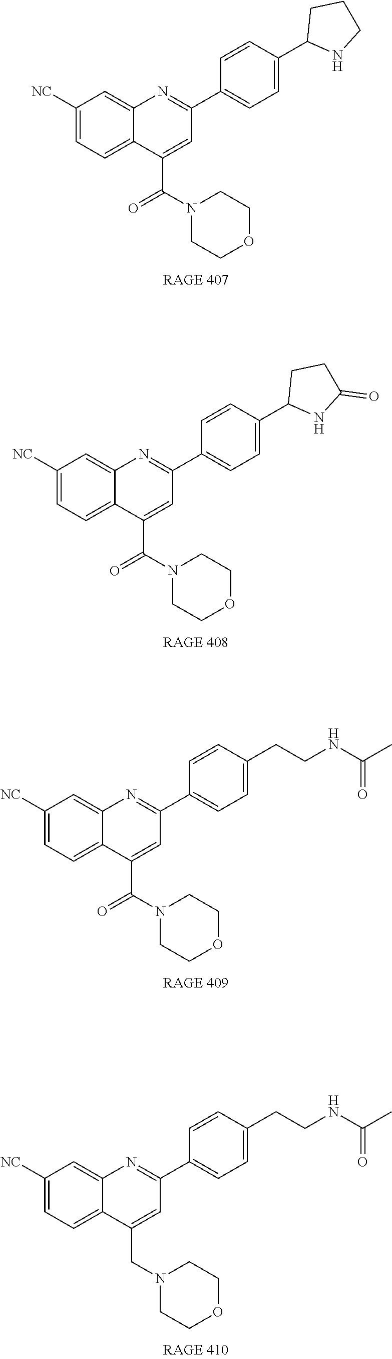 [SMILES: N#Cc1ccc2c(C(=O)N3CCOCC3)cc(-c3ccc(C4CCCN4)cc3)nc2c1 ; N#Cc1ccc2c(C(=O)N3CCOCC3)cc(-c3ccc(C4CCC(=O)N4)cc3)nc2c1 ; CC(=O)NCCc1ccc(-c2cc(C(=O)N3CCOCC3)c3ccc(C#N)cc3n2)cc1 ; CC(=O)NCCc1ccc(-c2cc(CN3CCOCC3)c3ccc(C#N)cc3n2)cc1]

or a pharmaceutically acceptable salt thereof.

In one embodiment of the compound of Formula (I), the compound has the structure:

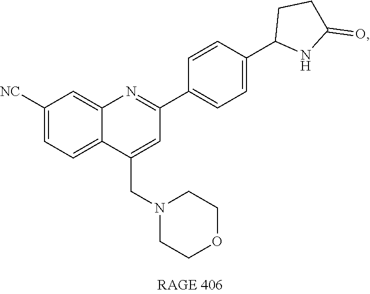 [SMILES: N#Cc1ccc2c(CN3CCOCC3)cc(-c3ccc(C4CCC(=O)N4)cc3)nc2c1]

or a pharmaceutically acceptable salt thereof.

In one embodiment of the compound of Formula (I), the compound is a racemic mixture of RAGE 406R and RAGE406S:

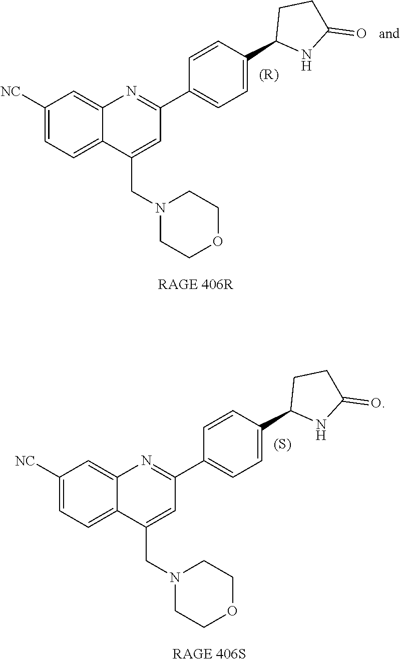

In one embodiment of the compound of Formula (I), the compound is RAGE 406R:

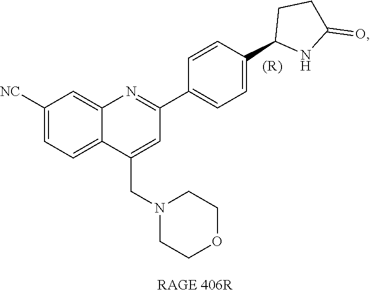

In one embodiment of the compound of Formula (I), the compound is Rage 406S:

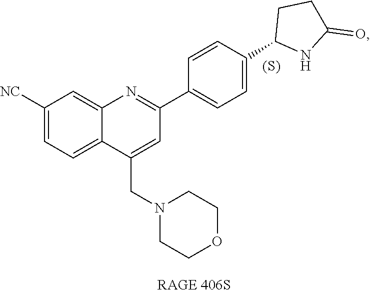

In another aspect, the present invention provides a pharmaceutical composition comprising any of the herein-described compounds or a pharmaceutically acceptable salt thereof and a pharmaceutically acceptable carrier.

In yet another aspect, the present invention provides a pharmaceutical dosage form comprising any of the herein-described compounds or pharmaceutical compositions or a pharmaceutically acceptable salt thereof and a pharmaceutically acceptable carrier.

### Pharmaceutical Compositions

When employed as pharmaceuticals, the compounds of this invention are typically administered in the form of a pharmaceutical composition. Such compositions can be prepared in a manner well known in the pharmaceutical art and comprise at least one active compound.

Generally, the compounds of this invention are administered in a therapeutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

The pharmaceutical compositions of this invention can be administered by a variety of routes including oral, rectal, intraocular, transdermal, subcutaneous, intravenous, intramuscular, intraperitoneal, interdermal, directly into cerebrospinal fluid, intratracheal, and intranasal. Depending on the intended route of delivery, the compounds of this invention are preferably formulated as either injectable or oral compositions or as salves, as lotions or as patches all for transdermal administration. In some embodiments, the compounds of this invention are formulated for local injection in suitable carriers to optimize tissue specific delivery.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the active compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10%) by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10%) by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention. The compounds of this invention can also be administered by a transdermal device. Accordingly, transdermal administration can be accomplished using a patch either of the reservoir or porous membrane type, or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compounds of this invention can also be administered locally to the eye for the treatment of diabetic neuropathy. Suitable compositions include those administrable by eye drops, injections or the like. In the case of eye drops, the composition can also optionally include, for example, ophthalmologically compatible agents such as isotonizing agents, buffering agents, surfactants, stabilization agents, and other ingredients. For injection, the compound can be provided in an injection grade saline solution, in the form of an injectable liposome solution, slow-release polymer system or the like.

The compounds of this invention can also be administered in sustained release forms or from sustained release drug delivery systems. A description of representative sustained release materials can be found in Remington's Pharmaceutical Sciences.

The following formulation examples illustrate representative pharmaceutical compositions of this invention. The present invention, however, is not limited to the following pharmaceutical compositions.

### Formulation 1—Tablets

A compound of the invention is admixed as a dry powder with a dry gelatin binder in an approximate 1:2 weight ratio. A minor amount of magnesium stearate is added as a lubricant. The mixture is formed into 240-270 mg tablets (80-90 mg of active amide compound per tablet) in a tablet press.

### Formulation 2—Capsules

A compound of the invention is admixed as a dry powder with a starch diluent in an approximate 1:1 weight ratio. The mixture is filled into 250 mg capsules (125 mg of active amide compound per capsule).

### Formulation 3—Liquid

A compound of the invention (125 mg), sucrose (1.75 g) and xanthan gum (4 mg) are blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

### Formulation 4—Tablets

A compound of the invention is admixed as a dry powder with a dry gelatin binder in an approximate 1:2 weight ratio. A minor amount of magnesium stearate is added as a lubricant. The mixture is formed into 450-900 mg tablets (150-300 mg of active amide compound) in a tablet press.

### Formulation 5—Injection

A compound of the invention is dissolved or suspended in a buffered sterile saline injectable aqueous medium to a concentration of approximately 5 mg/ml.

### Formulation 6—Topical

Stearyl alcohol (250 g) and a white petrolatum (250 g) are melted at about 75° C. and then a mixture of a compound of the invention (50 g) methylparaben (0.25 g), propylparaben (0.15 g), sodium lauryl sulfate (10 g), and propylene glycol (120 g) dissolved in water (about 370 g) is added and the resulting mixture is stirred until it congeals.

### Methods of Treatment

Types 1 and 2 diabetes are on the rise in the United States and world-wide (International Diabetes Federation. (2012) IDF Diabetes Atlas (5th edn), International Diabetes Federation, Brussels, Belgium; Patterson et al., “Trends in childhood type 1 diabetes incidence in Europe during 1989-2008: evidence of non-uniformity over time in rates of increase,” Diabetologia 55:2142-2147 (2012); Lipman et al., “Increasing Incidence of Type 1 Diabetes in Youth: Twenty years of the Philadelphia Pediatric Diabetes Registry,” Diabetes Care 36:1597-1603 (2013)). Type 1 diabetes incidence is also increasing world-wide in adults, which can lead to a significant reduction in lifespan (Lancet Diabetes Endocrinol. 2022 October; 10 (10): 741-760). The long-term consequences of diabetes ensue from the direct and indirect effects of hyperglycemia. Diabetes attacks the macro- and microvasculature and is well-established as a leading cause of heart attacks and stroke, blindness, renal failure, amputations, and peripheral neuropathies. The strong epidemiological links between diabetes and Alzheimer's disease raise the possibility that devastating loss of quality and duration of life in the form of irreversible chronic disease often accompany diabetes. Despite significant advances in the treatment of hyperglycemia, definitive means to prevent the complications of diabetes are not yet on the immediate horizon. Indeed, rigorous control of hyperglycemia, particularly in older individuals, may be fraught with significant sequelae, such as striking hypoglycemia, seizures, cardiac ischemia and death (Nathan et al., “Medical management of hyperglycemia in type 2 diabetes: a consensus algorithm for the initiation and adjustment of therapy: a consensus statement of the American Diabetes Association and the European Association for the Study of Diabetes,” Diabetes Care 3:193-203 (2009); (UKPDS), U.P.D.S.G. Intensive blood glucose control with sulphonylureas or insulin compared with conventional treatment and risk of complications in patients with type 2 diabetes (UKPDS 33). UK Prospective Diabetes Study (UKPDS) Group. Lancet, pp. 837-853 (1998); The Diabetes Control and Complications Trial Research Group (1993) The effect of intensive treatment of diabetes on the development and progression of long term complications in insulin-dependent diabetes mellitus. The Diabetes Control and Complications Trial Research Group. New Engl. J. Med. p. 977-986).

The products of nonenzymatic glycation and oxidation of proteins and lipids, the advanced glycation endproducts (AGEs), form and accumulate to accelerated degrees in hyperglycemia (Frye et al., “Role of the Maillard reaction in aging of tissue proteins. Advanced glycation end product-dependent increase in imidazolium cross-links in human lens proteins,” J. Biol. Chem. 273:18714-18719 (1998)). AGEs may be detected in the plasma, urine, skin and other tissues of diabetic subjects and their presence has been linked to the development of complications of diabetes. AGEs impart their effects in part by non-receptor mediated mechanisms, such as by cross-linking of the body's proteins, particularly those that are long-lived such as in basement membranes. AGEs also exert their effects by receptor-dependent mechanisms; the chief receptor for AGE is the receptor for AGE or RAGE. Extensive evidence reveals that expression of RAGE, a member of the immunoglobulin superfamily of cell surface molecules, is increased in animal model and human diabetic tissues, such as in the macro- and microvascular tissues. RAGE is a multi-ligand receptor and the finding that RAGE binds at least certain members of the proinflammatory S100/calgranulin family and high mobility group box 1 (HMGB1) indicate that inflammatory mechanisms contribute integrally to the pathogenesis of complications. Indeed, non-AGE RAGE ligands also accumulate in human and animal model diabetic tissues (Yan et al., “Tempering the wrath of RAGE: an emerging therapeutic strategy against diabetic complications, neurodegeneration, and inflammation,” Ann. Med. 41:408-422 (2009); Yan et al., “The RAGE axis: a fundamental mechanism signaling danger to the vulnerable vasculature,” Circ. Res. 106:842-853 (2010)). Once thought highly unlikely, the role of inflammation in at least certain forms/stages of diabetic complications is now widely appreciated. Pharmacological and genetic approaches by multiple laboratories, working independently, have provided very strong support for roles for RAGE in the pathogenesis of diabetic complications. For example, administration of antibodies to RAGE or soluble RAGE (the latter the extracellular ligand binding domain of RAGE) or genetic deletion of RAGE significantly reduces accelerated diabetic atherosclerosis in mice; ischemia/reperfusion injury in the diabetic hearts; pathological and functional indices of nephropathy; pathological and functional indices of neuropathy; and improves wound healing in diabetic animals (Yan et al., “Tempering the wrath of RAGE: an emerging therapeutic strategy against diabetic complications, neurodegeneration, and inflammation,” Ann. Med. 41:408-422 (2009); Yan et al., “The RAGE axis: a fundamental mechanism signaling danger to the vulnerable vasculature,” Circ. Res. 106:842-853 (2010)).

Accumulating evidence reveals that levels of soluble RAGEs (cell surface cleaved RAGE and the endogenous secretory (splice variant)) may be biomarkers of diabetes and its complications in human subjects; levels of soluble RAGEs appear to be modulated by therapeutic interventions, thereby raising the significance of measuring these forms of circulating RAGE.

In this direct context, the inventors and others demonstrated that the cytoplasmic domain of RAGE is essential for the impact of RAGE ligand-RAGE interaction in modulation of gene expression and generation of vascular and inflammatory cell dysfunction. The cytoplasmic domain of RAGE does not appear to exert its downstream signaling impact simply by endogenous phosphorylation; hence, the inventors sought to test the premise that intracellular binding effectors were essential to bind to the RAGE tail and thus facilitate engagement of intracellular signaling pathways. Toward that end, the inventors performed a yeast-two-hybrid assay using the RAGE tail as “bait.” From this experimental work, the inventors discovered and published in 2008 that the cytoplasmic domain of RAGE interacts with the forming molecule, DIAPH1 and that DIAPH1 is required for the impact of RAGE signaling in multiple cell types such as smooth muscle cells, macrophages, cardiomyocytes and endothelial cells (Hudson et al., “Interaction of the RAGE cytoplasmic domain with diaphanous-1 is required for ligand-stimulated cellular migration through activation of Rac1 and Cdc42,” J. Biol. Chem. 283:34457-34468 (2008); Rai et al., “Signal transduction in receptor for advanced glycation end products (RAGE): solution structure of C-terminal rage (cTRAGE) and its binding to mDial,” J. Biol. Chem. 287:5133-5144 (2012); Xu et al., Advanced glycation end product (AGE)-receptor for AGE (RAGE) signaling and up-regulation of Egr-1 in hypoxic macrophages,” J. Biol. Chem. 285:23233-23240 (2010); Toure et al., “Formin mDial mediates vascular remodeling via integration of oxidative and signal transduction pathways,” Circ. Res. 110:1279-1293 (2012), Zhu et al., Biochim Biophys Acta Biomembr. 2019 January; 1861 (1): 43-49, and Syed et al, Eur Biophys J. 2018 January; 47 (1): 39-48).

Fundamental observations link DIAPH1 to the pathological indices of RAGE signal transduction directly relevant to diabetic complications. Thus, modulating the interaction between RAGE and DIAPH1 is desirable for treating diseases and conditions where RAGE is implicated.

Further to the above, it will be appreciated that the compounds described herein act as modulators of RAGE binding to its intracellular ligands (e.g., DIAPH1) and thereby reduce or prevent the activation of NF-κB regulated genes, such as the cytokines IL-1 and TNF-α, and minimize the generation of oxidative stress. The ability of the compounds described herein to antagonize or inhibit the binding of physiological ligands to the intracellular tail of RAGE renders them well suited to use as therapeutic agents for treating or managing diseases or conditions related to RAGE activity. More particularly, the quinoline compounds described herein may be used to treat, for example, diabetes complications, inflammation, neurodegeneration, obesity, cancer, ischemia/reperfusion injury, cardiovascular disease, Alzheimer's Disease, and other diseases understood to be related to RAGE activity. Such compounds may be used to impair downstream signaling events resulting from, for example, AGE-RAGE interaction, which contributes to diabetic complications, S100A12/EN-RAGE/calgranulin-RAGE interaction, which contributes to inflammatory diseases, β-amyloid-RAGE interaction, which contributes to Alzheimer's Disease, and high mobility group box 1 (HMGB1)-RAGE interaction, which contributes to, e.g., inflammation and cancer.

### Diabetes and Diabetes Complications

Further to the above, the quinoline compounds described herein are useful for managing and/or treating complications associated with diabetes. Nonenzymatic glycoxidation of macromolecules results in the formation of advanced glycation endproducts (AGEs). The term AGEs refers to a heterogeneous group of compounds generated through the non-enzymatic glycation or glycoxidation of proteins, lipids, and nucleic acids. More particularly, AGEs are the result of a series of complex biochemical reactions that involve the formation of Amadori products, glyceraldehyde-3-phosphate, and the reactive carbonyl methylglyoxal (MG). See, for example, Manigrasso et al. (2014, Trends in Endocrin Metab 25:15-22); the entire content of which, including references cited therein, is incorporated herein by reference. Nonenzymatic glycoxidation of macromolecules is known to be enhanced in the presence of hyperglycemia and other conditions associated with systemic or local oxidant stress. It is also known to be enhanced in renal failure and at sites of inflammation, and amongst other locales associated with neurodegeneration, obesity, and cancer. Schmidt et al. (1995, Nature Med. 1:1002-1004), for example, have shown that AGEs accumulate generally in the vasculature and tissues of patients with diabetes. Other research has demonstrated that AGEs also accumulate in the vasculature focally, as observed in the joint amyloid composed of AGE-P2-microglobulin found in patients with dialysis-related amyloidosis (Abedini et al. 2013, FEBS Lett 587:1119-1127; Miyata et al. 1993, J. Clin. Invest. 92:1243-1252; Miyata et al. 1996, J. Clin. Invest. 98:1088-1094). AGE production is also directly accelerated by hyperglycemia. AGE formation is also frequently associated with an increase in reactive oxygen species (ROS) (Fu et al. 1994, Diabetes 43:676-683). Although AGEs accumulate slowly in both plasma and tissues during aging (Brownlee et al. 1988, N Engl J Med 318:1315-1321; Hallam et al. 2010, Aging Cell 9:776-784; Schleicher et al. 1997, J Clin Inv 99:457-468), they are markedly increased in patients with diabetes (Makita et al. 1991, N Engl J Med 325:836-842).

Suitable animal models in which to study diabetes complications are known in the art and are described in, for example, Manigrasso et al. (2014, Trends in Endocrin Metab 25:15-22); Stirban et al. (2014, Molecular Metabolism 3:94-108); Johnson et al. (2014, EJNMMI Res 4:26); Tekabe et al. (2014, Int J Mol Imaging Article Id 695391); Kaida et al. (2013, Diabetes 62:3241-3250); Tekabe et al. (2013, EJNMMi Res 3:37); Calcutt et al. (2009, Nat Rev Drug Discov 8:417-429); Dauch et al. (2013, J Neuroinfl animation 10:64); Juranek et al. (2013, Diabetes 62:931-943): Singh et al. (2014, Korean J Physiol Pharmacol 18:1-14); Ramasamy et al. (2012, Vascular Pharmacol 57:160-167); Montagnani (2008, Br J Pharmacol 154:725-726); Nakamura et al. (1993, Am J Pathol 143:1649-1656); Lin et al. (2003, Atherosclerosis 168:213-220); Hofmann et al. (2002, Diabetes 51:2082-2089); Lin et al. (2002, Atherosclerosis 163:303-311); Vlassara et al. (1992, Proc Natl Acad Sci 89:12043-12047); Brownlee et al. (1986, Science 232:1629-1632); Li et al. (1996, Proc Natl Acad Sci 93:3902-3907); Park et al. (1998, Nature Med 4:1025-1031); Kislinger et al. (2001, Arteriosclerosis, Thrombosis, and Vascular Biology 21:905-910); Bucciarelli et al. (2002, Circulation 106:2827-2835); Wendt et al. (2006, Atherosclerosis 185:70-77); the entire content of each of which is incorporated herein by reference.

### Diabetic Complications—Heart

More particularly, animal models of human diabetes involving diabetic complications of the heart include those involving ex vivo isolated perfused heart ischemia/reperfusion, left anterior descending coronary artery ligation, and cardiac autonomic neuropathy. References describing such models are known in the art and described in, for example, Stables et al. (2014, Autonom Neurosci 177:746-80), Bucciarelli et al. (2000, Circulation (Supplement) 102: #563, II-1 17), and Aleshin et al. (2008, Am J Physiol Heart Circ Physiol 294: H1823-H1832); the entire content of each of which is incorporated herein by reference.

### Diabetic Complications—Kidney

More particularly, animal models of human diabetes involving diabetic complications of the kidney include OVE26 mice, streptozotocin induced animals, Db/db mice, and nephrectomy. References describing such models are known in the art and described in, for example, Kaur et al. (2014, Inflammopharmacology 22:279-293), Reiniger et al. (2010, Diabetes 59:2043-2054), and Wendt et al. (2003, American Journal of Pathology 162:1123-1137); the entire content of each of which is incorporated herein by reference.

### Diabetic Complications—Retinopathy

More particularly, animal models of human diabetes involving diabetic complications leading to retinopathy include streptozotocin induced animals, Db/db mice, and Akita mice. References describing such models are known in the art and described in, for example, Lai et al. (2013, J Diabetes Res 013:106594) and Barile et al. (2005, Invest Ophthalmol Vis Sci 46:2916-2924); the entire content of each of which is incorporated herein by reference.

### Diabetic Complications—Neuropathy

More particularly, animal models of human diabetes involving diabetic complications leading to neuropathy include Swiss Webster mice, Db/db mice, and Sciatic nerve transection/crush. References describing such models are known in the art and described in, for example, Juranek et al. (2010, Biochem Insights 2010:47-59), Juranek et al. (2013, Diabetes 62:931-943), Islam (2013, J Diabetes Res 2013:149452); the entire content of each of which is incorporated herein by reference.

Animal models of diabetes in general include streptozotocin induced animals, Akita mice, Db/db mice, and Ob/ob mice. These animal models are known in the art and described in, for example, Park et al. (1998, Nature Medicine 4:1025-1031), Wendt et al. (2006, Atherosclerosis 185:70-77), Wang et al. (2014, Curr Diabetes Rev 10:131-145), and Acharjee et al. (2013, Can J Diabetes 37:269-276); the entire content of each of which is incorporated herein by reference.

### Immune/Inflammatory Responses

The quinoline compounds described herein are envisioned as useful for treating inflammation. In that inflammation is a common feature underlying all of the diseases and conditions described herein, it is reasonable to expect that these compounds will also be efficacious in the context of, for example, diabetes complications, obesity, cancer, ischemia/reperfusion injury, cardiovascular disease, neurodegeneration, Alzheimer's Disease, cystic fibrosis, multiple sclerosis, rheumatoid arthritis, psoriasis, atopic dermatitis, and eczema.

As alluded to above, RAGE is a receptor for many members of the SlOO/calgranulins, a family of closely related calcium-binding polypeptides that accumulate at sites of chronic immune/inflammatory responses, such as those observed in cystic fibrosis and rheumatoid arthritis. RAGE, moreover, is known to mediate the proinflammatory effects of SlOO/calgranulins on a variety of cells, including lymphocytes and mononuclear phagocytes. Indeed, RAGE-ligand interactions with, e.g., proinflammatory SlOO/calgranulins, high mobility group box 1 (HMGB1), and/or AGEs are implicated as having a pivotal role in the inflammatory cascade in general. See, for example, Ramasamy et al. (2012, Vascular Pharmacol 57:160-167); Andersson et al. (2011, Annu Rev Immunol 29:139-162); the entire content of each of which, including references cited therein, is incorporated herein by reference. Studies using in vitro models and in animal models of the delayed-type hypersensitivity (DTH) response, colitis in IL-10 null mice, collagen-induced arthritis, and experimental autoimmune encephalitis models further underscore the fundamental role of RAGE-ligand interactions in various inflammatory diseases including rheumatoid arthritis and multiple sclerosis.

RAGE is also been implicated in inflammatory diseases of the skin such as but not limited to psoriasis, atopic dermatitis, and eczema. Psoriasis may, moreover, be accompanied by arthropathic symptoms that are similar to those seen in rheumatoid arthritis. High levels of proinflammatory cytokines, particularly IL-1 and IL-8, are detected in psoriatic lesions. IL-8 is a chemotactic factor for neutrophils, which are known to synthesize and secrete SlOO proteins. As indicated herein above, SI 00 proteins are RAGE ligands, which interaction leads to the propagation of immune and inflammatory responses that contribute and lead to a variety of diseases/conditions described herein. Psoriasin (S100A7), a member of the SI 00 gene family, is a secreted protein isolated from psoriatic skin. Linkage of psoriasis genetic susceptibility to distinct overexpression of SI 00 proteins in the skin has, furthermore, been demonstrated (Semprini et. al. 2002, Hum. Genet. 111:310-3). The compounds described herein are therefore envisioned as therapeutic agents for psoriasis in light of their ability to inhibit RAGE mediated downstream signaling.

### High Mobility Group Box 1 (HMGB1)

HMGBl, which is also known as amphoterin, has dual activities. It was originally characterized as a structural protein localized to the nucleus where it functions to stabilize DNA structure and modulate transcriptional activity (Stros et al. 2010, Biochem Biophys Acta 1799:101-113). HMGBl was also later discovered to be an actively secreted cytokine, produced by macrophages and other inflammatory cells during the innate immune response to invasion (Wang et al. 1999, Science 285:248-251). Like other members of the proinflammatory cytokine family, biologically active HMGBl can be expressed on the plasma membrane or released by activated inflammatory cells to accumulate in vivo during infection and injury. HMGBl acts as an effector molecule capable of altering the metabolic and immunological activities of hematopoietic, epithelial, and neuronal cells. The breadth of its effector functions is reflected in its known activities, which include significant roles in fever, anorexia, acute-phase responses, and vascular leakage syndrome. HMGBl acts in synergy with other cytokines and pathogen-derived molecules in these diseases/conditions. The contribution of HMGBl to these and other pathological conditions is underscored by the numerous demonstrations that administration of agents that specifically inhibit HMGBl activity (antibodies, antagonist proteins, release inhibitors) to animals with ischemia and inflammatory diseases interrupts the progression of tissue injury and suppresses inflammatory responses in treated animals. See, Andersson et al. (2011, Annu Rev Immunol 29:139-162) for a review).

The available evidence thus demonstrates that HMGB 1 is a general mediator of inflammation, implicated in a plethora of inflammatory and autoimmune diseases. In that HMGB1 is a ligand for RAGE, these findings underscore the role of RAGE as a general mediator of inflammation and render apparent that targeting RAGE activity with the intent to inhibit downstream signaling therefrom has significant promise and use of the compounds described herein for the treatment of subjects afflicted with diseases/conditions characterized by inflammation and/or autoimmunity would attenuate clinical signs and symptoms of inflammation in such subjects.

Animal models of autoimmunity/inflammation include those involving delayed type hypersensitivity, rheumatoid arthritis, systemic lupus erythematosis, ulcerative colitis, Crohn's disease, psoriasis, Behcet's syndrome, Type 1 diabetes, vasculitis, glomerulonephritis, and sarcoidosis. Such animal models are known in the art and described in, for example, Hofmann et al. (1999, Cell 97:889-901), Hofmann et al. (2002, Genes and Immunity 3:123-135), Webb et al. (2014, Biochem Pharmacol 87:121-130), Sakata et al. (2012, Exp Diabetes Res 2012:256707), Goyal et al. (2014, Inflammopharmacology 22:219-233), Lu et al. (2014, Life Sci 108 (1): 1-6), Starr et al. (2014, Aging Dis 5:126-136); the entire content of each of which is incorporated herein by reference.

### Obesity

Animal models of human obesity are known in the art and involve feeding mice a 45% high fat diet or a 60% high fat diet. Such models are described in, for example, Song et al. (2014, Diabetes 63 (6): 1948-1965) and Aydin et al. (2014, Nutrition 30:1-9); the entire content of each of which is incorporated herein by reference.

### Cancer

Abnormal expression of RAGE and its ligands has been reported in a number of cancers, including prostatic, colorectal, pancreatic, lung, and oral squamous cell cancers. It is, moreover, thought that the interaction of RAGE with its ligands contributes to cancer invasion and metastasis. The interaction between RAGE and HMGBl triggers the activation of key cell signaling pathways, such as NF-κB, p38, p44/42 MAPKs, and activation of these pathways contributes to cancer progression and metastasis (Sims et al. 2010, Annu Rev Immunol 28:367-388; Sparvero et al. 2009, J Transl Med 7:17; Lodgson et al. 2007, Curr Mol Med 7:777-789; Kuniyasu et al. 2003, Oncol Rep 10:445-448; Kuniyasu et al. 2003, Int J Cancer 104:722-727; Sasahira et al. 2005, Virchows Arch 446:41 1-415; Kuniyasu et al. 2005, Am J Pathol 166:751-760; Kuniyasu et al. 2004, Pathobiology 71:129-136; Sasahira et al. 2007, Virchows Arch 450:287-295; Kuniyasu et al. 2002, J Pathol 196:163-170; the entire content of each of which is incorporated herein by reference). Further to the above, Rai et al. (2012, J Exp Med 209:2339-2350) and Arumugam et al. (2012, Clin Cane Res 18:4356-4364), for example, describe animal model systems in which the contribution of RAGE to various cancers has been investigated and validated.

Further to the above, RAGE and its ligand HMGB 1 are believed to play an important role in prostate cancer. Indeed, Zhao et al. (2014, Am J Cancer Res 4:369-377) addressed the significance of these effector molecules in a retrospective study designed to investigate the expression of RAGE and HMGB 1 and their clinical impact on prostate cancer progression and prognosis. The expression of RAGE and FIMGB 1 was assessed by immunohistochemistry in cancer lesions from 85 confirmed prostate cancer cases. Zhao et al. demonstrated that there is a strong correlation between RAGE and FnVIGB 1 expression (PO.OOl) and the expression of RAGE, FIMGB 1 and their co-expression were all associated with advanced tumor clinical stage (P<0.05 for all). RAGE expression was also associated with the prostate specific antigen (PSA) level (P=0.014). Co-expression of RAGE and FnVIGB 1 was also associated with poor overall survival in patients with stage III and IV prostate cancer (P=0.047). These results suggest that the expression of RAGE and HMGB 1 is associated with progression and poor prognosis of prostate cancer. RAGE and HMGB 1 are, therefore, proposed to be molecular targets for novel forms of therapy for prostate cancer.

### Tumors/Tumorigenesis

Animal models for various forms of human cancers are known in the art and include those recapitulating aspects of human lung cancer, melanoma, colon cancer, pancreatic cancer, and breast cancer and bio-models of cancer for in silico screening. Such animal models are known in the art and are described in, for example, Taguchi et al. (2000, Nature 405:354-360), Arumugam et al. (2004, Journal of Biological Chemistry 279:5059-5065), Huang et al. (2006, Surgery 139:782-788), Huang et al. (2006, Surgery 139:782-788), Fuentes et al. (2007, Dis Colon Rectum 50:1230-1240), Arumugam et al. (2012, Clin Cancer Res 18:4356-4364), Yu et al. (2014, J Gastric Cancer 14:67-86), Fleet (2014, Am J Physiol Gastrointest Liver Physiol. 307 (3): G249-59), Lindner (2014, Semin Oncol 41:146-155), Wang et al. (2014, Biofabrication 6 (2): 022001), Budhu et al. (2014, Curr Opin Genet Dev 24:46-51, 2014); the entire content of each of which is incorporated herein by reference.

### Ischemia/Reperfusion Injury

In, for example, animal models of hind limb ischemia in mice with or without diabetes, suppressing RAGE ligands has led to improvement of angiogenic response to limb ischemia. See, for example, Tamarat et al. (2003, Proc Natl Acad Sci 100:14); Goova et al. (2001, Am J Pathol 159:513-525); Tekabe et al. (2010, J Nuc Med 51:92-97); Tekabe et al. (2013, EJNMMi Res 3:37); Bucciarelli et al. (2008, Diabetes 57:1941-1951); Shang et al. (2010, PLOS 5: el0092); Ma et al. (2009, J Cell Mol Med 13:1751-1764); the entire content of each of which is incorporated herein by reference.

### Erectile Dysfunction

Relaxation of the smooth muscle cells in the cavernosal arterioles and sinuses results in increased blood flow into the penis, raising corpus cavernosum pressure to culminate in penile erection. Nitric oxide is considered the principle stimulator of cavernosal smooth muscle relaxation (See Wingard et al. (2001, Nature Medicine 7:119-122). In that RAGE activation produces oxidants via an NADH oxidase-like enzyme (Yan et al. 1994, J. Biol. Chem. 269:9889-9887), it is thought to suppress nitric oxide circulation. Inhibiting activation of RAGE signaling pathways is, therefore, predicted to attenuate oxidant generation. Inhibition of RAGE-mediated activation of Rho-kinases is also predicted to enhance and stimulate penile erection independently of nitric oxide. Accordingly, compounds such as those described herein that act to inhibit downstream RAGE signaling may be used to advantage to promote and facilitate penile erection.

### Respiratory Diseases

Patients with chronic obstructive pulmonary disease exhibit increased RAGE expression in the lung and elevated soluble RAGE levels in the bronchial alveolar fluid (Yan et al. 2003, Nature Med 9:287-293; Miniati et al. 2011, Respir Res 12:37). Increased RAGE receptor and ligand levels have also been detected in asthmatic patients (Watanabe et al. 2010, Respir Med 105:519-525), indicating an active role for RAGE in lung inflammation. See also Wu et al. (2013, Mol Cell Biochem 380:249-257); Sukkar et al. (2012, Br J Pharmacol 167: Inline).

Furthermore, in severe exacerbations of asthma there is an intense, mechanistically heterogeneous inflammatory response involving neutrophil and eosinophil accumulation and activation. Neutrophils are, moreover, a significant source of SI 00 proteins, key ligands for RAGE implicated in the propagation of the immune response and inflammation as described herein above. Accordingly, inhibitors of RAGE downstream signaling would be expected to be efficacious in the treatment of asthma. In that the propagation step in the immune response in the lung driven by S100-RAGE interaction is thought to lead to the activation and/or recruitment of inflammatory cells, such as neutrophils, which are significant sources of damaging proteases in chronic obstructive pulmonary diseases such as emphysema, the compounds described herein that act as RAGE inhibitors can be used to treat chronic obstructive pulmonary diseases.

Animal models for assessing the therapeutic potential of compounds described herein in the context of respiratory disease (e.g., asthma) are presented in, for example, Akirav et al. (2014, PLOS One9: e95678); and Constant et al. (2002, J Clin Invest 110:1441-1448); the entire content of each of which is incorporated herein by reference.

### Amyloidoses

Compounds described herein are also envisioned as useful for treating amyloidoses and Alzheimer's Disease (AD). RAGE is known to bind β-sheet fibrillar material and deposition of amyloid has been shown to enhance expression of RAGE. The brains of AD patients exhibit increased expression of RAGE in neurons and glia (Yan et al. 1996, Nature 382:685-691). Binding of Aβ-RAGE on microglia activates these cells, as reflected by increased motility and expression of cytokines, whereas binding of Aβ-RAGE on neurons initially activates the cells, but ultimately leads to cytotoxicity. Inhibition of RAGE-amyloid interaction decreases expression of cellular RAGE and cell stress markers (as well as NF-κB activation) and diminishes amyloid deposition (Yan et al. 2000, Nat. Med. 6:643-651). These findings suggest that a role for RAGE-amyloid interaction exists, both with respect to perturbation of cellular properties in an environment enriched for amyloid at early stages of disease and progressively during the course of disease as amyloid accumulates.

### Neurodegeneration

Animal models of human neurodegenerative diseases are known and include mouse models of Alzheimer's Disease, humanized mouse models of Amyotrophic lateral sclerosis, and mouse models of Huntington's disease. Such animal models are described in, for example, Millington et al. (2014, Biomed Res Inst 2014:309129), Yan et al. (1996, Nature 382:685-691), Yan et al. (1997, Proc. Natl. Acad. Sci. 94:5296-5301), Bard et al. (2014, J Biomol Screen 19:191-204), Neha et al. (2014, Life Sci 109 (2): 73-86), and Turner et al. (2013, Amyotroph Lateral Scler Frontotemporal Degener. 14 Suppl 1:19-32); the entire content of each of which is incorporated herein by reference.

### Atherosclerosis

Examples of animal models of human atherosclerotic disease include apolipoprotein E null mice and Low Density Lipoprotein Receptor null mice. See, for example, Kapourchali et al. (2014, World J Clin Cases 2:126-132), Harja et al. (2008, J. Clin. Invest. 1118:183-194), Nagareddy et al. (2013, Cell Metab 17:695-708); the entire content of each of which is incorporated herein by reference.

In light of that which is understood in the art and described herein regarding the prominent role of RAGE in diseases/conditions characterized by acute and chronic inflammation, methods are presented herein for treating such diseases/conditions, including but not limited to diabetic complications, ischemia, skin inflammation (e.g., psoriasis and atopic dermatitis), lung inflammation (e.g., asthma and chronic obstructive pulmonary disease), vascular permeability, nephropathy, atherosclerosis, retinopathy, Alzheimer's Disease, erectile dysfunction, and tumor invasion and/or metastasis, which methods comprise administering to a subject in need thereof a compound described herein in a therapeutically effective amount. In a particular embodiment, at least one compound described herein is utilized, either alone or in combination with one or more known therapeutic agents. In a further particular embodiment, the present invention provides a method for treating RAGE mediated human diseases, wherein treatment alleviates one or more symptoms resulting from that disorder, the method comprising administration to a human in need thereof a therapeutically effective amount of a compound described herein.

In vitro assays relating to RAGE-mediated diseases and animal model systems thereof are described in US2012/0088778, US2010/0254983, US2010/0119512, U.S. Pat. No. 7,361,678, WO2007/089616, and US2010/0249038, the entire content of each of which is incorporated herein by reference.

Further to the above, the present compounds are modulators of interaction between RAGE and RAGE ligands and are used as therapeutic agents for the treatment of conditions in mammals that are causally related or attributable to RAGE activity. Accordingly, the compounds and pharmaceutical compositions of this invention find use as therapeutics for preventing and/or treating a variety of conditions related to, for example, diabetes complications in mammals, including humans.

In a method of treatment aspect, this invention provides a method of treating a mammal susceptible to or afflicted with a condition associated with diabetes complications, Alzheimer's disease, cancers, arthiritis, nephropathy, acute and chronic inflammation, retinopathy, atherosclerosis, erectile dysfunction, tumor invasion and metastasis, and others, which method comprises administering an effective amount of one or more of the pharmaceutical compositions just described.

In additional method of treatment aspects, this invention provides methods of treating a mammal susceptible to or afflicted with an inflammatory condition causally related or attributable to RAGE activity. Such condition and disorders include, without limitation, diabetes and its complications, impaired wound healing, peripheral vascular disease and associated complications, obesity, Alzheimer's disease, cancers, arthritis, nephropathy, acute and chronic inflammation, retinopathy, atherosclerosis, cardiovascular disease, erectile dysfunction, tumor invasion and metastases, neuropathy, cardio- and cerebrovascular ischemia/reperfusion injury, heart attack, stroke, myocardial infarction, ischemic cardiomyopathy, renal ischemia, sepsis, pneumonia, infection, liver injury, liver damage, Amyotrophic lateral sclerosis, neuropathy infection, allergy, asthma, organ damage from pollutants, amyloidoses asthma, pollution-associated tissue damage, skin disorders, colitis, skin aging, lupus, and others. Such methods comprise administering an effective condition-treating or condition-preventing amount of one or more of the pharmaceutical compositions just described.

As a further aspect of the invention there is provided the present compounds for use as a pharmaceutical especially in the treatment or prevention of the aforementioned conditions and diseases. Also provided herein is the use of the present compounds in the manufacture of a medicament for the treatment or prevention of one of the aforementioned conditions and diseases.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prevention and/or treatment of long-term conditions, such as, e.g., arthritis, diabetes, or asthma, the regimen for treatment usually stretches over many months or years, so oral dosing is preferred for patient convenience and tolerance. With oral dosing, one to five and especially two to four and typically three oral doses per day are representative regimens. Using these dosing patterns, each dose provides from about 0.01 to about 20 mg/kg of the compound of the invention, with preferred doses each providing from about 0.1 to about 10 mg/kg and especially about 1 to about 5 mg/kg.

Transdermal doses are generally selected to provide similar or lower blood levels than are achieved using injection doses. Modes of administration suitable for mucosal sites are also envisioned herein and include without limitation: intra-anal swabs, enemas, intranasal sprays, and aerosolized or vaporized compounds and/or compositions for delivery to the lung mucosa. One of skill in the art would choose an appropriate delivery mode/s based on a variety of parameters, including the organ or tissue site in a patient with a disease or condition that is most severely affected by the disease or condition.

When used to prevent the onset of an inflammatory condition or autoimmune disorder, the compounds of this invention will be administered to a patient at risk for developing the condition or disorder, typically on the advice and under the supervision of a physician, at the dosage levels described above. Patients at risk for developing a particular condition generally include those that have a family history of the condition, or those who have been identified by genetic testing or screening to be particularly susceptible to developing the condition.

When used to treat diabetic eye disease, the compounds of this invention can be administered intraocularly.

The compounds of this invention can be administered as the sole active agent or they can be administered in combination with other agents, including other compounds that demonstrate the same or a similar therapeutic activity and are determined to safe and efficacious for such combined administration.

### EXAMPLES

The following examples illustrate specific aspects of the instant description. The examples should not be construed as limiting, as the examples merely provide specific understanding and practice of the embodiments and their various aspects.

### General Synthetic Procedures

The quinoline compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

All commercially available starting materials and solvents were of reagent grade and used without further purification. All reactions were conducted under an atmosphere of dry nitrogen unless otherwise specific. Thin layer chromatography (TLC) plates were visualised with ultraviolet light. Flash chromatography refers to column chromatography over silica gel (100-200 mesh) using glass columns. Alternatively, automated chromatography was performed using Biotage Isolera system with ultraviolet detection at 214, 254 or 280 nm and employing Biotage normal phase or reverse phase silica cartridges. Solvents used for samples are specified in the experimental procedures for each compound. The following system was used for liquid chromatography mass spectrometry (LC-MS): Agilent 1260 (quaternary pump), XBridge analytical column C18, 2.7 μm, 4.6×30 mm, 45° C., 1 μL injection volume, 1.8 mL/min. Mobile phase: Acetonitrile (0.05% FA)—water (0.05% FA), gradient changes from 5% acetonitrile to 95% acetonitrile in 1.0 min, hold 1.0 min, total 2.5 min. The following system was used for Waters UPLC (no mass spectrometry): Waters Acquity BEH C18, 2.1×50 mm, 1.7 μm, 0.5 mL/min, 45° C.; Mobile phase: Acetonitrile (0.05% TFA)-water (0.05% TFA); gradient: 5%-95% acetonitrile over 2 min then hold at 95% acetonitrile for 0.7 min. Nuclear magnetic resonance (NMR) spectra were measured with a Bruker spectrometer operating at 400 MHz (¹H), 376 MHz (¹⁹F) or 100 MHz (¹³C). Proton NMR data are reported with chemical shifts (8) in parts-per-million (ppm) relative to the residual signal of deuterated solvents.

### Example 1: Synthesis of Analog RAGE 283 According to the Invention

Compound RAGE 283 according to the present disclosure was prepared as shown in Scheme 1 and described below.

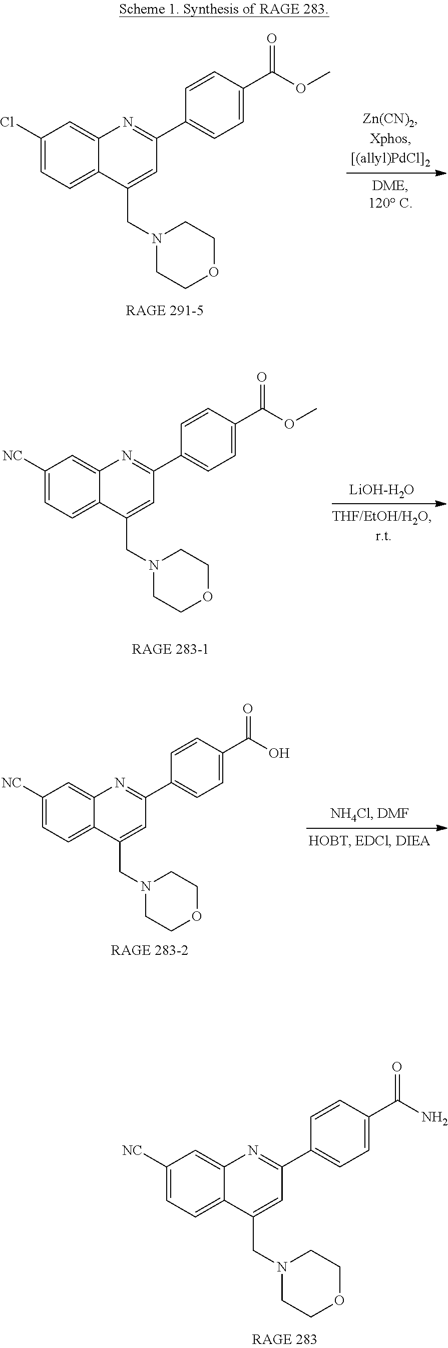 [SMILES: COC(=O)c1ccc(-c2cc(CN3CCOCC3)c3ccc(Cl)cc3n2)cc1 ; COC(=O)c1ccc(-c2cc(CN3CCOCC3)c3ccc(C#N)cc3n2)cc1 ; "LiOH-H2O" ; N#Cc1ccc2c(CN3CCOCC3)cc(-c3ccc(C(=O)O)cc3)nc2c1 ; N#Cc1ccc2c(CN3CCOCC3)cc(-c3ccc(C(N)=O)cc3)nc2c1]

### Example 1A: Preparation of Compound RAGE 283-1

A mixture of methyl 4-(7-chloro-4-(morpholinomethyl)quinolin-2-yl)benzoate (500 mg, 1.26 mmol), Zn(CN)₂ (295 mg, 2.52 mmol), Xphos (120 mg, 0.252 mmol) and [Pd(allyl)Cl]₂ (46 mg, 0.126 mmol) in DME (20 mL) was stirred at 120° C. for 24 h. The mixture reaction was filtered and washed with EtOAc (20 mL×3). The filtrate was concentrated in vacuo to give the crude, which was purified by silica gel chromatography (DCM/MeOH=20/1) to give RAGE 283-1 (400 mg, 82.0%) as a yellow solid. LC-MS [M+H]⁺: 387.2.

### Example 1B: Preparation of Compound RAGE 283-2

To a solution of methyl 4-(7-cyano-4-(morpholinomethyl)quinolin-2-yl)benzoate (400 mg, 1.03 mmol) in THF/EtOH/H₂O (5 mL/5 mL/5 mL) was added LiOH—H₂O (130 mg, 3.09 mmol). The resulting mixture was stirred at room temperature for 3 h. After the reaction was completed, the reaction mixture was acidified with 1M HCl until pH=3, concentrated in vacuo to give the crude product which was purified by silica gel chromatography (eluent: DCM/MeOH=10/1) to give RAGE 283-2 (350 mg, yield: 90.8%) as a white solid. LC-MS [M+H]⁺: 374.2.

### Example 1C: Preparation of Compound RAGE 283

A mixture of 4-(7-cyano-4-(morpholinomethyl)quinolin-2-yl)benzoic acid (150 mg, 0.4 mmol), NH₄Cl (106 mg, 2.0 mmol), EDCl (153 mg, 0.8 mmol), HOBT (108 mg, 0.8 mmol) and DIPEA (258 mg, 2.0 mmol) in DMF (10 mL) was stirred at 25° C. for 16 h. The reaction was poured into H₂O (30 mL) and extracted with EtOAc (30 mL×3), The combined organic layers were washed with brine, dried over Na₂SO₄, concentrated in vacuo to give the crude, which was purified by Prep-HPLC to give RAGE 283 (32 mg, yield: 21.4%) as a white solid. LC-MS [M+H]⁺: 373.3, Rt=1.440 min. Purity: 99.63 (214 nm), 99.67 (254 nm). NMR (400 MHz, DMSO-d6) δ 8.64 (s, 1H), 8.53 (d, J=8.9 Hz, 1H), 8.38 (s, 1H), 8.36 (s, 1H), 8.33 (s, 1H), 8.13 (s, 1H), 8.09 (s, 1H), 8.06 (s, 1H), 7.95-7.92 (m, 1H), 7.51 (s, 1H), 4.04 (s, 2H), 3.58-3.55 (m, 4H), 2.53-2.49 (m, 4H).

### Example 2: Synthesis of Analog RAGE 286 According to the Invention

Compound RAGE 286 according to the present disclosure was prepared as shown in Scheme 2 and described below.

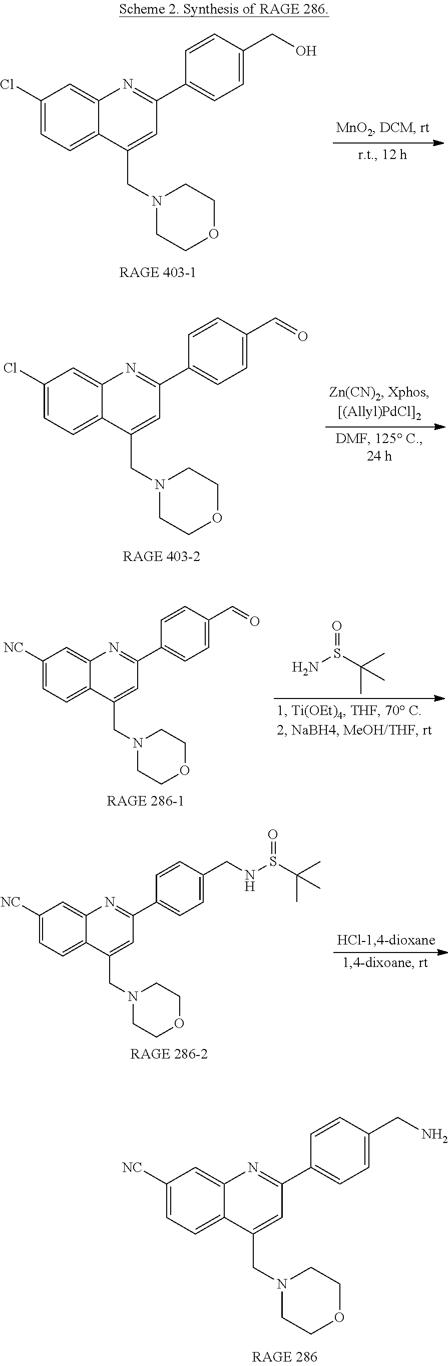

### Example 2A: Preparation of Compound RAGE 403-2

To a solution of (4-(7-chloro-4-(morpholinomethyl)quinolin-2-yl)phenyl) methanol (600 mg, 1.63 mmol) in CH₂Cl₂ (30 mL) was added MnO₂ (3048 mg, 16.3 mmol). The resulting mixture was stirred at room temperature for 18 h. The mixture reaction was filtered and washed with CH₂Cl₂ (30 mL×2). The filtrate was concentrated in vacuo to give the crude product, which was purified by silica gel column chromatography (eluent: DCM/MeOH=20/1) to give RAGE 403-2 (410 mg, yield: 68.7%) as yellow solid. LC-MS [M+H]⁺: 367.1.

### Example 2B: Preparation of Compound RAGE 286-1

A mixture of methyl 4-(7-chloro-4-(morpholinomethyl)quinolin-2-yl)benzaldehyde (410 mg, 1.12 mmol), Zn(CN)₂ (262 mg, 2.24 mmol), Xphos (107 mg, 0.224 mmol) and [Pd(allyl)Cl]₂ (41 mg, 0.112 mmol) in DMF (20 mL) was stirred at 125° C. for 24 h. After the reaction was completed, the reaction was poured into H₂O (50 ml) and extracted with EtOAc (40 ml×3), The combined organic layers were washed with brine, dried over Na₂SO₄, concentrated in vacuo to give the crude, which was purified silica gel chromatography (eluent: DCM/MeOH=20/1) to give RAGE 286-1 (300 mg, 75.1%) as a yellow solid. LC-MS [M+H]⁺: 358.2.

### Example 2C: Preparation of Compound RAGE 286-2

To a reaction mixture of 2-(4-formylphenyl)-4-(morpholinomethyl)quinoline-7-carbonitrile (300 mg, 0.84 mmol) and 2-methylpropane-2-sulfinamide (203 mg, 1.68 mol) in THF (20 mL) was added Ti(i-PrO)₄ (358 mg, 1.26 mmol). The resulting solution was stirred at 70° C. for 16 h. Then MeOH (4 mL) and NaBH₄ (96 mg, 2.52 mmol) was successively added at 0° C. and the solution was stirred for additional one hour at room temperature. The reaction was quenched with saturated aqueous NH₄Cl (sat. aq., 30 mL) and extracted with EtOAc (3×30 mL). The combined organic layers were washed with brine, dried over Na₂SO₄, concentrated in vacuo to give the crude, which was purified by silica gel chromatography (eluent: DCM/MeOH=30/1) to RAGE 286-2 (200 mg, yield: 51.5%) as yellow solid. LC-MS [M+H]⁺: 463.2.

### Example 2D: Preparation of Compound RAGE 286

To a reaction mixture of N-(4-(7-cyano-4-(morpholinomethyl)quinolin-2-yl)benzyl)-2-methylpropane-2-sulfinamide (200 mg, 0.42 mmol) in 1,4-dioxane (6 mL) was added HCl (3 mL, 4M solution in dioxane, 12 mmol). The reaction was stirred at room temperature for 0.5 h. The pH of reaction was adjusted to 8˜9 with NaHCO₃ (sat. aq.) and extracted with EtOAc (3×30 mL). The combined organic layers were washed with brine, dried over Na₂SO₄, concentrated in vacuo to give the crude, which was purified by Prep-HPLC to afford RAGE 286 (35 mg, yield: 22.6%) as white solid. LC-MS [M+H]⁺: 359.1, Rt=0.870 min. Purity: 100 (214 nm), 100 (254 nm). ¹H NMR (400 MHz, DMSO-d6) δ 8.60 (s, 1H), 8.52 (d, J=8.8 Hz, 1H), 8.35 (d, J=8.9 Hz, 2H), 8.29 (s, 1H), 8.27 (s, 1H), 7.95-7.92 (m, 1H), 7.66 (d, J=8.9 Hz, 2H), 4.12 (s, 2H), 4.02 (d, J=8 Hz, 2H), 3.59-3.55 (m, 4H), 2.53-2.49 (m, 4H).

### Example 3: Synthesis of Analog RAGE 289 According to the Invention

Compound RAGE 289 according to the present disclosure was prepared as shown in Scheme 3 and described below.

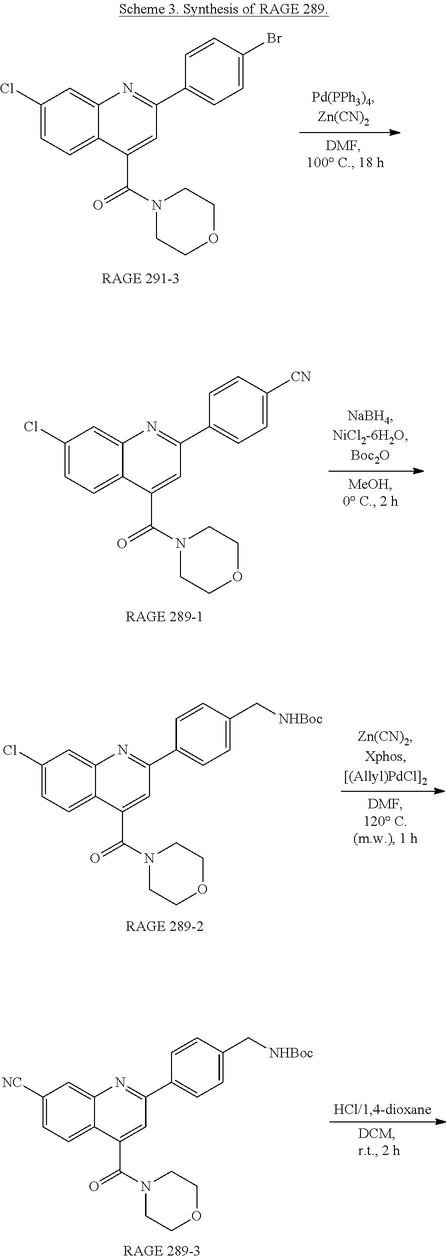 [SMILES: O=C(c1cc(-c2ccc(Br)cc2)nc2cc(Cl)ccc12)N1CCOCC1 ; N#Cc1ccc(-c2cc(C(=O)N3CCOCC3)c3ccc(Cl)cc3n2)cc1 ; CC(C)(C)OC(=O)NCc1ccc(-c2cc(C(=O)N3CCOCC3)c3ccc(Cl)cc3n2)cc1 ; CC(C)(C)OC(=O)NCc1ccc(-c2cc(C(=O)N3CCOCC3)c3ccc(C#N)cc3n2)cc1]

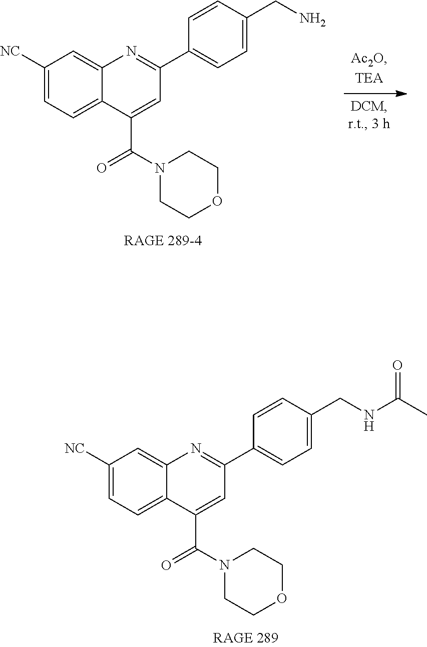 [SMILES: N#Cc1ccc2c(C(=O)N3CCOCC3)cc(-c3ccc(CN)cc3)nc2c1 ; CC(=O)NCc1ccc(-c2cc(C(=O)N3CCOCC3)c3ccc(C#N)cc3n2)cc1]

### Example 3A: Preparation of Compound RAGE 289-1

A mixture of (2-(4-bromophenyl)-7-chloroquinolin-4-yl)(morpholino)methanone (3.0 g, 6.95 mmol), Zn(CN)₂ (1.63 g, 13.9 mmol) and Pd(PPh₃)₄ (803 mg, 0.695 mmol) in DMF (50 mL) was stirred at 100° C. for 18 h. After the reaction was completed, the reaction was poured into H₂O (100 ml) and extracted with EA (80 ml×3), The combined organic layers were washed with brine, dried over Na₂SO₄, concentrated in vacuo to give the crude, which was purified silica gel chromatography (eluent: DCM/MeOH=20/1) to give RAGE 289-1 (2.4 g, 91.4%) as a yellow solid. LC-MS [M+H]⁺: 378.1.

### Example 3B: Preparation of Compound RAGE 289-2

To a reaction mixture of 4-(7-chloro-4-(morpholine-4-carbonyl)quinolin-2-yl)benzonitrile (500 mg, 1.32 mmol) and NiCl₂*6H₂O (150 mg, 0.66 mmol) in MeOH (10 mL) was added Boc₂O (576 mg, 2.64 mmol). The reaction was stirred at room temperature under N₂ for 30 min. The solution was then cooled to 0° C. and NaBH₄ (166 mg, 3.96 mmol) was added in portions. The resulting reaction was stirred at room temperature for another 2 hours. The reaction was quenched with saturated aqueous NH₄Cl (30 mL) and extracted with ethyl acetate (3×30 mL). The combined organic layers were washed with brine, dried over Na₂SO₄, concentrated in vacuo to give the crude, which was purified silica gel chromatography (eluent: DCM/MeOH=20/1) to give the RAGE 289-2 (230 mg, yield: 36.1%) as yellow solid. LC-MS [M+H]⁺: 482.2.

### Example 3C: Preparation of Compound RAGE 289-3

A mixture of tert-butyl (4-(7-chloro-4-(morpholine-4-carbonyl)quinolin-2-yl)benzyl)carbamate (230 mg, 0.477 mmol), Zn(CN)₂ (112 mg, 0.954 mmol), Xphos (45 mg, 0.095 mmol) and [Pd(allyl)Cl]₂ (18 mg, 0.048 mmol) in DMF (3 mL) was stirred at 120° C. with microwave for 1 hour. After the reaction was completed, the reaction was poured into H₂O (20 ml) and extracted with EtOAc (20 mL×3), The combined organic layers were washed with brine (30 mL), dried over Na₂SO₄, concentrated in vacuo to give the crude, which was purified silica gel chromatography (eluent: DCM/MeOH=20/1) to give RAGE 289-3 (190 mg, 84.3%) as a yellow solid. LC-MS [M+H]⁺: 473.2.

### Example 3D: Preparation of Compound RAGE 289-4

To a reaction mixture of tert-butyl (4-(7-cyano-4-(morpholine-4-carbonyl)quinolin-2-1)benzyl)carbamate (190 mg, 0.40 mmol) in DCM (6 mL) was added HCl (4M solution in dioxane, 3 mL, 12 mmol). The reaction was stirred at room temperature for 2 h. After completed, the reaction was concentrated, the pH of the residue was adjusted to 8˜9 with NaHCO₃ (sat. aq.) and extracted with DCM (3×30 mL). The combined organic layers were washed with brine (30 mL), dried over Na₂SO₄, concentrated in vacuo to give the crude, which was purified silica gel chromatography (eluent: DCM/MeOH=15/1) to give RAGE 289-4 (100 mg, yield: 66.8%) as white solid. LC-MS [M+H]⁺: 373.2, Rt=0.986 min. Purity: 97.94 (214 nm), 97.50 (254 nm). ¹H NMR (400 MHz, DMSO) δ 8.69 (s, 1H), 8.42-8.34 (m, 3H), 8.31 (s, 1H), 8.02 (d, J=8.5 Hz, 1H), 7.98-7.93 (m, 1H), 7.64 (d, J=8.2 Hz, 2H), 4.05 (s, 2H), 3.89 (s, 1H), 3.81-3.77 (m, 4H), 3.65 (s, 1H), 3.45 (s, 1H), 3.23 (s, 1H), 3.16 (s, 1H).

### Example 3E: Preparation of Compound RAGE 289

To a solution of 2-(4-(aminomethyl)phenyl)-4-(morpholine-4-carbonyl)quinoline-7-carbonitrile (100 mg, 0.27 mmol) in DCM (10 ml) was added Et₃N (82 mg, 0.81 mmol) at 0° C., then acetyl chloride (107 mg, 1.36 mmol) was added dropwise at 0° C. The resulting reaction was stirred at room temperature for 3 h. The reaction was quenched with H₂O (15 mL), extracted with EtOAc (15 mL×3). The combined organic layers were washed with brine (30 mL), dried over Na₂SO₄, concentrated in vacuo to give the crude, which was purified by Prep-HPLC to provide RAGE 289 (50 mg, 44.9%) as a white solid. LC-MS [M+H]⁺: 415.2, Rt=1.203 min. Purity: 100 (214 nm), 100 (254 nm). ¹H NMR (400 MHz, DMSO-d6) δ 8.69 (s, 1H), 8.47-8.44 (m, 1H), 8.35 (s, 1H), 8.31 (d, J=8.9 Hz, 2H), 8.01 (d, J=8.8 Hz, 1H), 7.95-7.92 (m, 1H), 7.46 (d, J=8.9 Hz, 2H), 4.35 (d, J=4.2 Hz, 2H), 3.92-3.90 (m, 1H), 3.80-3.70 (m, 3H), 3.37-3.22 (m, 2H), 3.22-3.15 (m, 2H), 1.91 (s, 3H).

### Example 4: Synthesis of Analog RAGE 290 According to the Invention

Compound RAGE 290 according to the present disclosure was prepared as shown in Scheme 4 and described below.

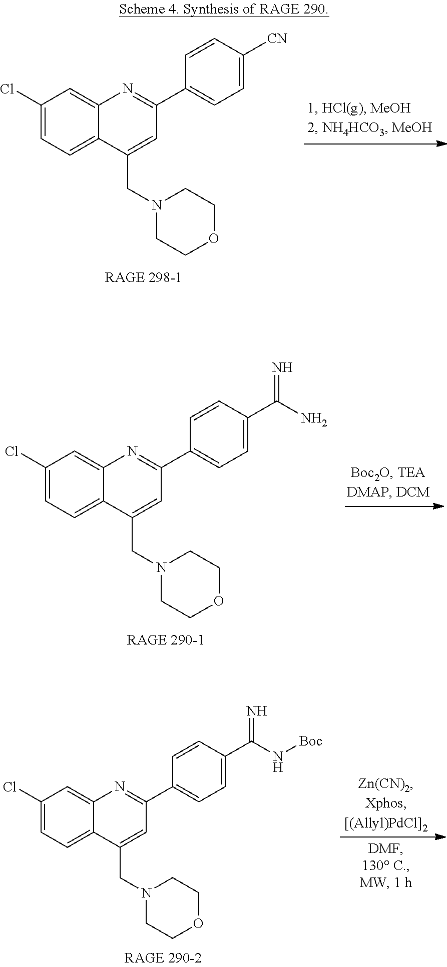 [SMILES: N#Cc1ccc(-c2cc(CN3CCOCC3)c3ccc(Cl)cc3n2)cc1 ; N=C(N)c1ccc(-c2cc(CN3CCOCC3)c3ccc(Cl)cc3n2)cc1 ; CC(C)(C)OC(=O)NC(=N)c1ccc(-c2cc(CN3CCOCC3)c3ccc(Cl)cc3n2)cc1]

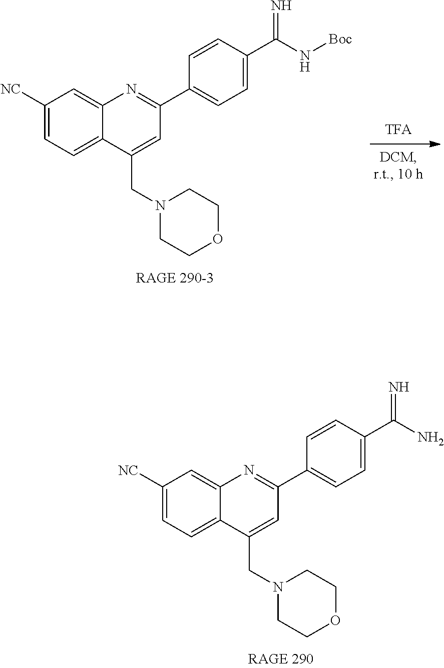 [SMILES: CC(C)(C)OC(=O)NC(=N)c1ccc(-c2cc(CN3CCOCC3)c3ccc(C#N)cc3n2)cc1 ; N#Cc1ccc2c(CN3CCOCC3)cc(-c3ccc(C(=N)N)cc3)nc2c1]

### Example 4A: Preparation of Compound RAGE 290-1

To a reaction mixture of 4-(7-chloro-4-(morpholinomethyl)quinolin-2-yl)benzonitrile (500 mg, 1.37 mmol) in MeOH (20 mL) was bubbled the HCl (gas) under ice bath. The reaction was stirred at room temperature for 3 h. Then the reaction solution was concentrated and dried under high vacuum. The resulting mixture was diluted with MeOH (20 mL), NH₄HCO₃ (216 mg, 2.74 mmol) was added thereto. The resulting mixture was stirred at room temperature for 16 h. After the reaction was completed, the reaction was concentrated in vacuo to give the crude product RAGE 290-1 (500 mg, yield: 95.5%) as yellow solid. LC-MS [M+H]⁺: 381.2.

### Example 4B: Preparation of Compound RAGE 290-2

To a solution of 4-(7-chloro-4-(morpholinomethyl)quinolin-2-yl)benzimidamide (500 mg, 1.31 mmol), DMAP (16 mg, 0.13 mmol) in DCM (20 mL) was added Et₃N (397 mg, 3.93 mmol) and Boc₂O (342 mg, 1.57 mmol). The resulting mixture was stirred at room temperature for 16 h. After the reaction was completed, the reaction was concentrated in vacuo to give the crude, which was purified by silica gel chromatography (eluent: EtOAc/PE=1/1) to provide RAGE 290-2 (350 mg, 55.4%) as yellow solid. LC-MS [M+H]⁺: 481.2.

### Example 4C: Preparation of Compound RAGE 290-3

A mixture of tert-butyl ((4-(7-chloro-4-(morpholinomethyl)quinolin-2-yl)phenyl) (imino)methyl)carbamate (350 mg, 0.728 mmol), Zn(CN)₂ (170 mg, 1.456 mmol), Xphos (70 mg, 0.146 mmol) and [Pd(allyl)Cl]₂ (27 mg, 0.073 mmol) in DMF (3 mL) was stirred at 130° C. for 1 h with microwave. After the reaction was completed, the reaction was poured into H₂O (20 mL) and extracted with EA (20 mL×3), The combined organic layers were washed with brine (30 mL), dried over Na₂SO₄, concentrated in vacuo to give the crude, which was purified silica gel chromatography (eluent: DCM/MeOH=20/1) to give RAGE 290-3 (150 mg, 43.7%) as a yellow solid. LC-MS [M+H]⁺: 472.2.

### Example 4D: Preparation of Compound RAGE 290

To a reaction mixture of tert-butyl ((4-(7-cyano-4-(morpholinomethyl)quinolin-2-yl)phenyl) (imino)methyl)carbamate (150 mg, 0.318 mmol) in DCM (10 mL) was added TFA (1 mL). The reaction was stirred at room temperature for 10 h. The pH of reaction was adjusted to 8˜9 with NaHCO₃ (sat. aq.) and extracted with DCM (3×30 mL). The combined organic layers were washed with brine (30 mL), dried over Na₂SO₄, concentrated in vacuo to give the crude, which was purified by Prep-HPLC to provide N-(4-(7-cyano-4-(morpholine-4-carbonyl)quinolin-2-yl)benzyl) acetamide (35.8 mg, yield: 30.3%) as white solid. LC-MS [M+H]⁺: 372.1, RT=0.867 min. Purity: 98.59 (214 nm), 98.09 (254 nm). ¹H NMR (400 MHz, DMSO-d6) δ 9.65 (s, 3H), 8.65 (d, J=4 Hz, 1H), 8.55-8.50 (m, 3H), 8.37 (s, 1H), 8.02 (d, J=8 Hz, 2H), 7.98-7.95 (m, 1H), 4.05 (s, 2H), 3.57-3.56 (m, 4H), 2.53-2.49 (m, 4H).

### Example 5: Synthesis of Analog RAGE 291 According to the Invention

Compound RAGE 291 according to the present disclosure was prepared as shown in Scheme 5 and described below.

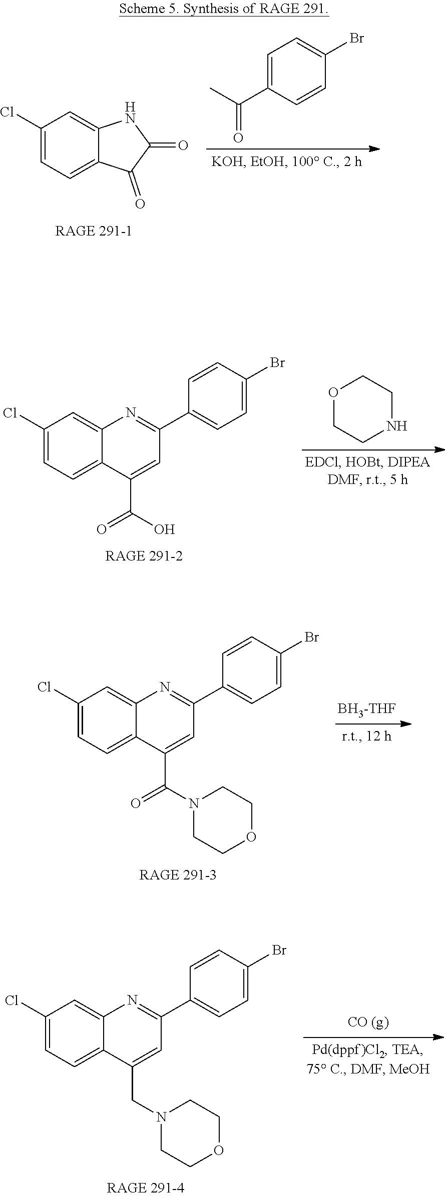

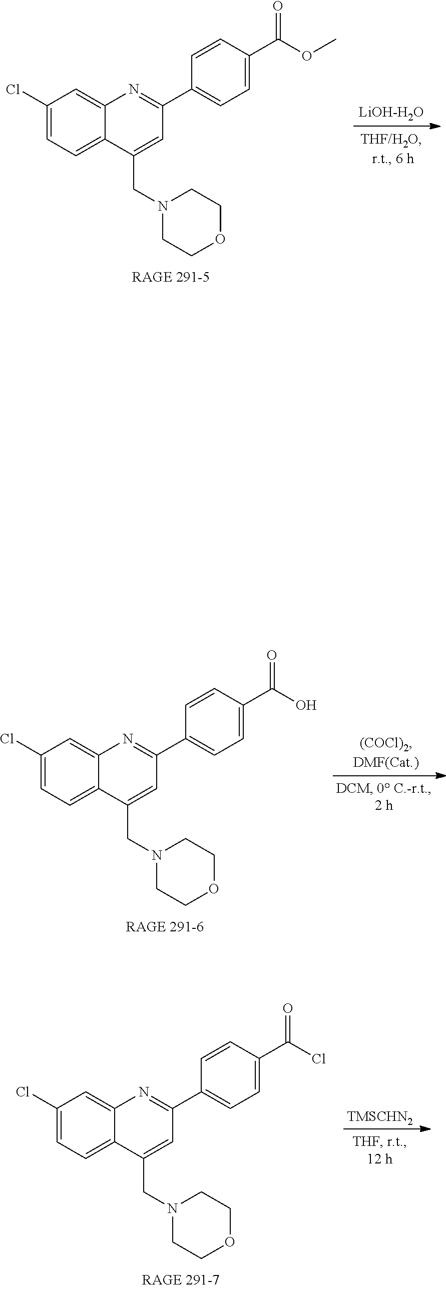 [SMILES: COC(=O)c1ccc(-c2cc(CN3CCOCC3)c3ccc(Cl)cc3n2)cc1 ; "LiOH-H2O" ; O=C(O)c1ccc(-c2cc(CN3CCOCC3)c3ccc(Cl)cc3n2)cc1 ; "(COCl)2, DMF(Cat.)" ; O=C(Cl)c1ccc(-c2cc(CN3CCOCC3)c3ccc(Cl)cc3n2)cc1 ; "TMSCHN2"]

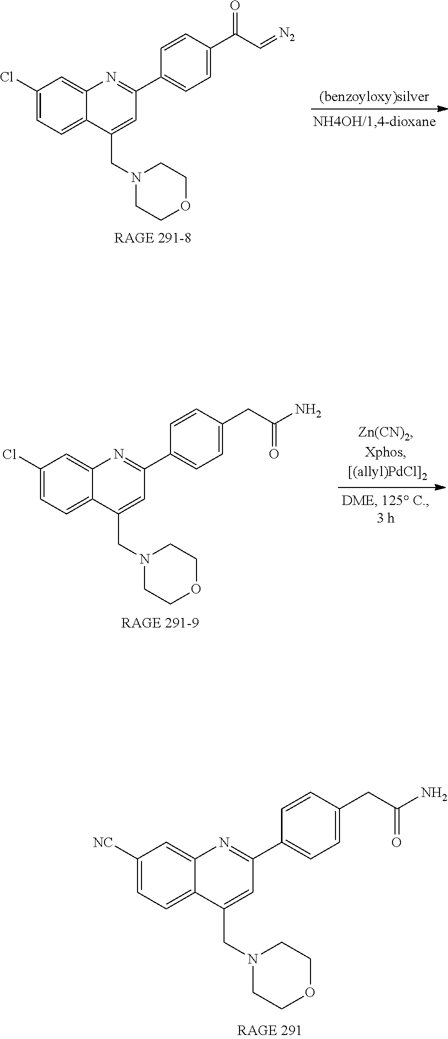

### Example 5A: Preparation of Compound RAGE 291-2

A solution of 1-(4-bromophenyl)ethan-1-one (49.3 g, 247.8 mmol) in EtOH (300 mL) was added to a solution of 6-chloroindoline-2,3-dione (30.0 g, 165.2 mmol) in 6 M aqueous KOH (200 mL) at 100° C. The reaction was monitored by LC-MS until the starting material consumed. Then the reaction mixture was concentrated to remove EtOH, then adjusted the pH to 4˜5 by HCl (6 N). The precipitate was filtered and dried to give RAGE 291-2 (40 g, 66.8%) as a red solid. LC-MS [M+H]⁺: 364.0

### Example 5B: Preparation of Compound RAGE 291-3

A solution of 2-(4-bromophenyl)-7-chloroquinoline-4-carboxylic acid (40 g, 110.3 mmol), morpholine (14.40 g, 165.5 mmol), EDCI (42.13 g, 220.6 mmol), HOBT (29.78 g, 220.6 mmol) and DIPEA (42.69 g, 330.9 mmol) in DMF (600 mL) was stirred at room temperature for 5 h. The reaction was poured into H₂O (800 mL) and extracted with EtOAc (500 mL×3), The combined organic layers were washed with brine (600 mL), dried over Na₂SO₄, concentrated in vacuo to give the crude, which was purified silica gel chromatography (eluent: EtOAc/PE=1/1) to provide RAGE 291-3 (39 g, 82.9%) as a yellow solid. LC-MS [M+H]⁺: 433.1.

### Example 5C: Preparation of Compound RAGE 291-4

To a solution of (2-(4-bromophenyl)-7-chloroquinolin-4-yl)(morpholino)methanone (39 g, 90.3 mmol) in THF (400 mL) was added BH₃-THF (1 M solution in THF, 270.9 mL, 270.9 mmol) at 0° C. The solution was stirred at room temperature for 12 h under N₂. The reaction was quenched by adding 200 mL MeOH at 0° C. The resulting mixture was heated to 50° C. for 0.5 h, then concentrated under vacuum to get crude product. The crude product was dissolved with water (50 mL) and extracted from water by EtOAc (50 mL×3). The combined organic layers were washed with brine (60 mL), dried over Na₂SO₄, concentrated in vacuo to give the crude, which was purified silica gel chromatography (eluent: DCM/MeOH=25/1) to provide RAGE 291-4 (25.0 g, yield 66.3%) as a yellow solid. LC-MS [M+H]⁺: 419.1.

### Example 5D: Preparation of Compound RAGE 291-5

To a mixture of 4-((2-(4-bromophenyl)-7-chloroquinolin-4-yl)methyl)morpholine (3.3 g, 7.9 mmol) and Et₃N (2.39 g, 23.7 mmol) in DMF/MeOH (15 mL/15 mL) was added Pd(dppf)Cl₂ (578 mg, 0.79 mmol). The resulting reaction mixture was stirred at 75° C. for 16 h under CO atmosphere. After cooling to room temperature, H₂O (100 mL) was added and the mixture was extracted with EtOAc (100 mL×3). The combined organic layers were washed with brine (100 mL), dried over anhydrous Na₂SO₄, and concentrated in vacuo to give the crude, which was purified by column chromatography (eluent: DCM/MeOH=30/1) to give RAGE 291-5 (1.3 g, yield: 41.5%) as a yellow solid. LC-MS [M+H]⁺: 397.2.

### Example 5E: Preparation of Compound RAGE 291-6

To a solution of methyl 4-(7-chloro-4-(morpholinomethyl)quinolin-2-yl)benzoate (1.3 g, 3.28 mmol) in THF/H₂O (10 mL/5 mL) was added LiOH—H₂O (413 mg, 9.84 mmol). The resulting mixture was stirred at room temperature for 3 h. After the reaction was completed, the reaction mixture was concentrated to remove, the pH of the aqueous layer was acidified with HCl (1 N) to 3. The yellow solid was precipitated and filtered to give RAGE 291-6 (1.2 g, yield: 95.7%) as a yellow solid. LC-MS [M+H]⁺: 383.1.

### Example 5F: Preparation of Compound RAGE 291-7

To a cooled (0° C.) and stirred solution of 4-(7-chloro-4-(morpholinomethyl)quinolin-2-yl)benzoic acid (1.2 g, 3.13 mol) and DMF (46 mg, 0.31 mol) in DCM (30 mL) was added (COCl)₂ (478 mg, 3.76 mmol) dropwise. The reaction mixture was warm to room temperature and stirred for 0.5 h. The reaction was concentrated to give the crude product RAGE 291-7 (1.25 g, yield: 99.4%) as a yellow solid. LC-MS [M+H]⁺: 397.2.

### Example 5G: Preparation of Compound RAGE 291-8

To a cooled (0° C.) and stirred solution of 4-(7-chloro-4-(morpholinomethyl)quinolin-2-yl)benzoyl chloride (1.25 g, 3.11 mol) in MeCN (30 mL) was added dropwise TMSCH₂N₂ (6.2 ml, 6.22 mol). The reaction mixture was stirred at room temperature for 16 h. After the reaction was completed, the reaction was concentrated to give the crude product RAGE 291-8 (1.1 g, yield: 86.8%) as a yellow solid. LC-MS [M+H]⁺: 407.2.

### Example 5H: Preparation of Compound RAGE 291-9

To a solution of 1-(4-(7-chloro-4-(morpholinomethyl)quinolin-2-yl)phenyl)-2-diazoethan-1-one (1.1 g, crude) in dioxane/NH₄OH (20 mL/5 mL) was added silver benzoate (618 mg, 2.7 mmol). The reaction mixture was heated at 120° C. for 1 h. The mixture was cooled to room temperature and filtered through a short pad of celite. The filtrated was diluted with ethyl acetate (30 mL×3), and washed with brine (40 mL), dried over Na₂SO₄, and concentrated. The residue was purified by chromatography (eluent: PE/EtOAc=2/1)) to give RAGE 291-9 (400 mg, yield: 37.4%) as yellow solid. LC-MS [M+H]⁺: 396.2.

### Example 5I: Preparation of Compound RAGE 291

A mixture of methyl 2-(4-(7-chloro-4-(morpholinomethyl)quinolin-2-yl)phenyl) acetamide (300 mg, 0.76 mmol), Zn(CN)₂ (178 mg, 1.52 mmol), Xphos (72 mg, 0.15 mmol) and [Pd(Allyl)Cl]₂ (29 mg, 0.08 mmol) in DME (10 mL) was stirred at 125° C. for 3 h. The mixture reaction was filtered and washed with EtOAc (20 mL×3). The filtrate was concentrated in vacuo to give the crude, which was purified by Prep-HPLC to give RAGE 291 (26 mg, 8.9%) as a white solid. LC-MS [M+H]⁺: 387.2, Rt=0.893 min. Purity: 98.76 (214 nm), 98.83 (254 nm). ¹H NMR (400 MHz, MeOD-d4) δ 8.56-8.48 (m, 2H), 8.19-8.15 (m, 3H), 7.76 (d, J=8.8 Hz, 1H), 7.52 (d, J=8.9 Hz, 2H), 4.05 (s, 2H), 3.65-3.60 (m, 4H), 3.30 (s, 2H), 2.61-2.57 (m, 4H).

### Example 6: Synthesis of Analog RAGE 292 According to the Invention

Compound RAGE 292 according to the present disclosure was prepared as shown in Scheme 6 and described below.

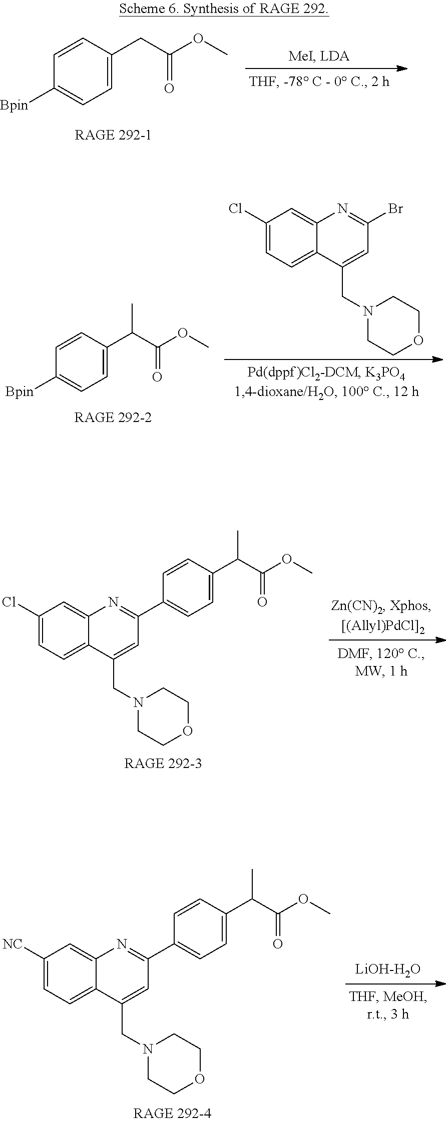

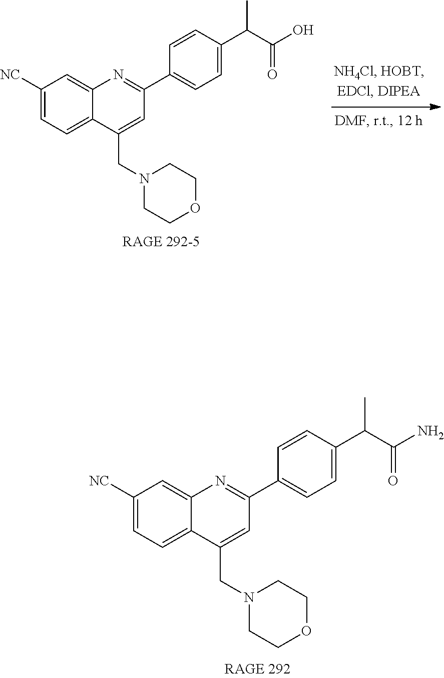 [SMILES: CC(C(=O)O)c1ccc(-c2cc(CN3CCOCC3)c3ccc(C#N)cc3n2)cc1 ; CC(C(N)=O)c1ccc(-c2cc(CN3CCOCC3)c3ccc(C#N)cc3n2)cc1]

### Example 6A: Preparation of Compound RAGE 292-2

To a solution of methyl 2-(4-(4,4,5,5-tetramethyl-1,3,2-dioxaborolan-2-yl)phenyl)acetate (1.0 g, 3.62 mmol) in THF (20 mL) was added LDA (3.6 mL, 1 M solution in hexanes, 3.6 mmol) dropwise. After stirred at −78° C. for 1 h, a solution of Mel (1.03 g, 7.24 mmol) in THF (5 mL) was added thereto slowly. The reaction was warm to room temperature and stirred for 2 hours. After completed, the reaction was quenched with saturated aqueous NH₄Cl (40 mL) and extracted with EtOAc (3×40 mL). The combined organic layers were washed with brine (60 mL), dried over Na₂SO₄, concentrated in vacuo to give the crude product, which was purified silica gel chromatography (eluent: EA/PE=1/10) to give RAGE 292-2 (850 mg, yield: 80.9%) as colorless oil. LC-MS [M+H]⁺: 320.2.

### Example 6B: Preparation of Compound RAGE 292-3

A mixture of methyl 2-(4-(4,4,5,5-tetramethyl-1,3,2-dioxaborolan-2-yl)phenyl)propanoate (450 mg, 1.55 mmol), 4-((2-bromo-7-chloroquinolin-4-yl)methyl)morpholine (530 mg, 1.55 mmol), K₃PO₄ (986 mg, 4.65 mmol) and Pd(dppf)Cl₂ (117 mg, 0.16 mmol) in dioxane/H₂O (20 mL/2 mL) was stirred at 100° C. for 12 h. After completed, the mixture reaction was filtered and washed with EtOAc (20 mL×3). The filtrate was concentrated in vacuo to give the crude, which was purified silica gel chromatography (eluent: DCM/MeOH=20/1) to give RAGE 292-3 (450 mg, 68.3%) as a yellow solid. LC-MS [M+H]⁺: 425.2

### Example 6C: Preparation of Compound RAGE 292-4

A mixture of methyl 2-(4-(7-chloro-4-(morpholinomethyl)quinolin-2-yl)phenyl)propanoate (450 mg, 1.06 mmol), Zn(CN)₂ (248 mg, 2.12 mmol), Xphos (100 mg, 0.21 mmol) and [Pd(Allyl)Cl]₂ (40 mg, 0.11 mmol) in DMF (5 mL) was stirred at 120° C. by microwave for 1 h. After the reaction was completed, the reaction was poured into H₂O (20 mL) and extracted with EtOAc (20 mL×3), The combined organic layers were washed with brine (50 mL), dried over Na₂SO₄, concentrated in vacuo to give the crude, which was purified silica gel chromatography (eluent: DCM/MeOH=20/1) to give RAGE 292-4 (320 mg, 72.7%) as a yellow solid. LC-MS [M+H]⁺: 416.2.

### Example 6D: Preparation of Compound RAGE 292-5

To a solution of methyl 2-(4-(7-cyano-4-(morpholinomethyl)quinolin-2-yl)phenyl)propanoate (400 mg, 0.77 mmol) in THF/MeOH/H₂O (5 mL/5 mL/5 mL) was added LiOH—H₂O (97 mg, 2.31 mmol). The resulting mixture was stirred at room temperature for 3 h. After the reaction was completed, the reaction mixture was acidified with 1M HCl until pH=3, concentrated in vacuo to give the crude product, which was purified silica gel chromatography (eluent: DCM/MeOH=10/1) to give RAGE 292-5 (280 mg, yield: 90.6%) as a white solid. LC-MS [M+H]⁺: 402.2.

### Example 6E: Preparation of Compound RAGE 292

A mixture of 2-(4-(7-cyano-4-(morpholinomethyl)quinolin-2-yl)phenyl)propanoic acid (280 mg, 0.70 mmol), NH₄Cl (186 mg, 3.5 mmol), EDCI (267 mg, 1.4 mmol), HOBT (189 mg, 1.4 mmol) and DIPEA (451 mg, 3.5 mmol) in DMF (10 mL) was stirred at room temperature for 16 h. The reaction was poured into H₂O (30 mL) and extracted with EA (30 mL×3), The combined organic layers were washed with brine (30 mL), dried over Na₂SO₄, concentrated in vacuo to give the crude, which was purified by Prep-HPLC to give RAGE 292 (100 mg, yield: 35.8%) as a white solid. LC-MS [M+H]⁺: 401.2, Rt=0.993 min. Purity: 100 (214 nm), 100 (254 nm). ¹H NMR (400 MHz, DMSO-d6) δ 8.59 (d, J=1.2 Hz, 1H), 8.50 (d, J=8.0 Hz, 1H), 8.24 (s, 2H), 8.22 (s, 1H), 7.92-7.90 (m, 1H), 7.53 (d, J=8.0 Hz, 2H), 7.47 (s, 1H), 6.90 (s, 1H), 4.03 (s, 2H), 3.68-3.59 (m, 1H), 3.58-3.57 (m, 4H), 2.52-2.50 (m, 4H), 1.38 (d, J=8.0 Hz, 3H).

### Example 7: Synthesis of Analog RAGE 293 According to the Invention

Compound RAGE 293 according to the present disclosure was prepared as shown in Scheme 7 and described below.

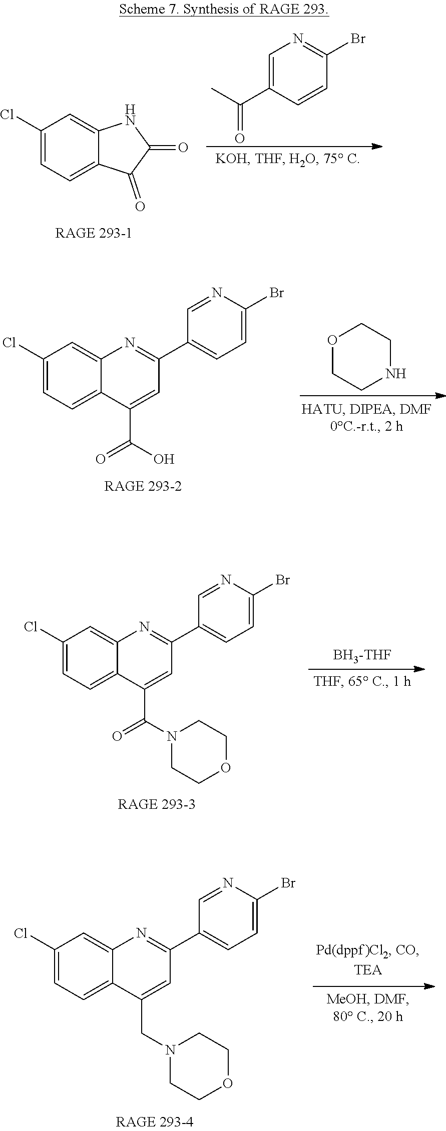

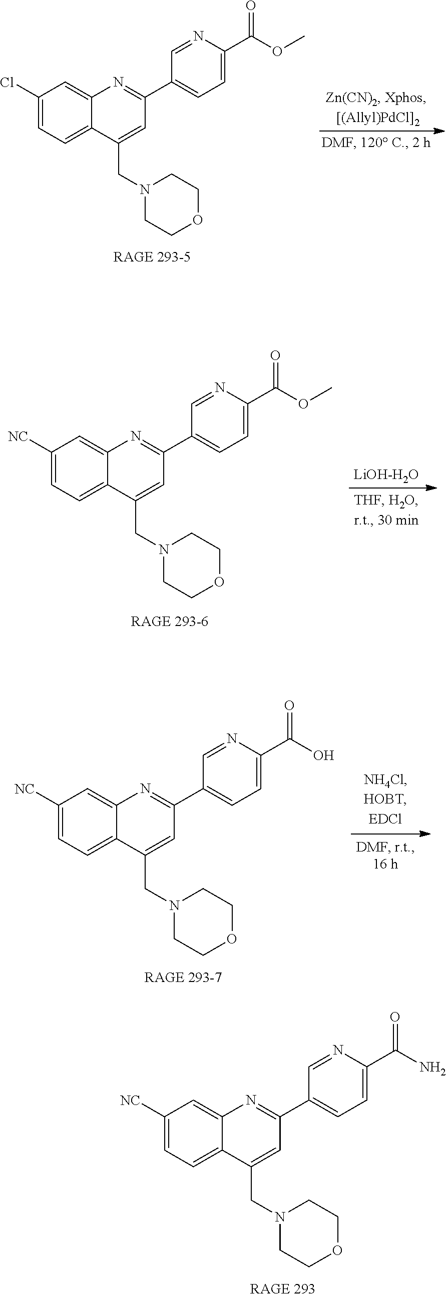 [SMILES: COC(=O)c1ccc(-c2cc(CN3CCOCC3)c3ccc(Cl)cc3n2)cn1 ; COC(=O)c1ccc(-c2cc(CN3CCOCC3)c3ccc(C#N)cc3n2)cn1 ; "LiOH-H2O" ; N#Cc1ccc2c(CN3CCOCC3)cc(-c3ccc(C(=O)O)nc3)nc2c1 ; N#Cc1ccc2c(CN3CCOCC3)cc(-c3ccc(C(N)=O)nc3)nc2c1]

### Example 7A: Preparation of Compound RAGE 293-2

To a solution of 1-(6-bromopyridin-3-yl)ethan-1-one (6.6 g, 33.1 mmol) in THF (80 mL) was added to a solution of 6-chloroindoline-2,3-dione (4.0 g, 22.1 mmol) in 6 M aqueous KOH (20 mL) at 75° C. The reaction was monitored by LC-MS until the starting material consumed. Then the reaction mixture was concentrated to remove THE, the pH of the residue was adjusted to 4˜5. The precipitate was filtered and dried to give the desired RAGE 293 (6.4 g, yield: 80%) as a red solid. LC-MS [M+H]⁺: 363.2.

### Example 7B: Preparation of Compound RAGE 293-3

A solution of 2-(6-bromopyridin-3-yl)-7-chloroquinoline-4-carboxylic acid (6.4 g, 17.7 mmol), morpholine (2.3 g, 26.6 mmol), HATU (10.1 g, 26.6 mmol) and DIPEA (6.8 g, 53.1 mmol) in DMF (50 mL) was stirred at room temperature for 2 h. The reaction was diluted with H₂O (200 mL), and then extracted with EtOAc (100 mL×3). The combined organic phase was washed with brine (100 mL), dried over Na₂SO₄, filtered and concentrated in vacuo to provide the crude, which was purified with silica gel chromatography (eluent: PE/EtOAc=1/1) to give desired RAGE 293-3 (6.5 g, yield: 85.5%) as a yellow solid. LC-MS [M+H]⁺: 432.2.

### Example 7C: Preparation of Compound RAGE 293-4

To a solution of (2-(6-bromopyridin-3-yl)-7-chloroquinolin-4-yl)(morpholino)methanone (4.0 g, 9.3 mmol) in THF (80 mL) was added BH₃-THF (1 M solution in THF, 27.9 mL, 27.9 mmol) at 0° C. The solution was heated to 20° C. for 8 h under N₂. The reaction was quenched by adding 20 mL of MeOH at 0° C. The mixture was heated to 65° C. for 1 h. The resulting solution was concentrated under vacuum to get crude product. The crude product was dissolved into water (50 mL) and pH was adjusted to round 10 by adding 1 M NaOH. The product was extracted with EtOAc (50 mL×3). The combined organic layers were washed with brine, dried over anhydrous Na₂SO₄ and concentrated to get crude desired RAGE 293-4 (2.6 g, crude) as a yellow solid, which was used into next step without further purification. LC-MS [M+H]⁺: 418.2

### Example 7D: Preparation of Compound RAGE 293-5

To a solution of 4-(7-chloro-4-(morpholinomethyl)quinolin-2-yl) aniline (1.2 g, 2.9 mmol) in MeOH/DMF (20 ml/20 ml) was added Pd(dppf)Cl₂ (219 mg, 0.3 mmol) and TEA (879 mg, 8.7 mmol). Mixture was stirred at 80° C. for 20 h under CO atmosphere. The reaction was quenched with H₂O (60 mL), extracted with EtOAc (30 mL×3). The combined organic layers were washed with brine (30 mL), dried over Na₂SO₄, concentrated in vacuo to give the crude, which was purified with silica gel chromatography (petroleum ether/EtOAc=1/1) to give desired RAGE 293-5 (300 mg, 26%) as a yellow solid. LC-MS [M+H]⁺: 398.2.

### Example 7E: Preparation of Compound RAGE 293-6

To a solution of (2-(6-bromopyridin-3-yl)-7-chloroquinolin-4-yl)(morpholino)methanone (300 mg, 0.8 mmol), Zn(CN)₂ (278 mg, 2.4 mmol), Xphos (38 mg, 0.08 mmol) and [Pd(allyl)Cl]₂ (29 mg, 0.08 mmol) in DMF (10 mL) was stirred at 120° C. for 2 h. The mixture reaction was filtered and washed with EtOAc (20 mL×3). The filtrate was concentrated in vacuo to give the crude, which was purified silica gel chromatography (DCM/MeOH=10/1) to give desired RAGE 293-6 (190 mg, 61%) as a yellow solid. LC-MS [M+H]⁺: 389.2.

### Example 7E: Preparation of Compound RAGE 293-7

To a solution of methyl 5-(7-cyano-4-(morpholinomethyl)quinolin-2-yl) picolinate (190 mg, 0.5 mmol) and LiOH (63 mg, 1.5 mmol) in THF/H₂O (5 mL/5 mL) was stirred at room temperature for 0.5 h. The organic layer was evaporated, and the water phase was acidified with 1 N HCl to pH=3 and extracted with EtOAc (30 mL×3). The combined organic layers were washed with brine, dried over Na₂SO₄, and concentrated to give desired RAGE 293-7 (100 mg, crude) as a yellow solid. LC-MS [M+H]⁺: 375.2.

### Example 7F: Preparation of Compound RAGE 293

To a solution of 5-(7-cyano-4-(morpholinomethyl)quinolin-2-yl) picolinic acid (100 mg, crude), NH₄Cl (79.5 mg, 1.5 mmol), EDCI (87 mg, 0.45 mmol) and HOBT (61 mg, 0.45 mmol) in DMF (10 mL) was added DIPEA (116 mg, 0.9 mmol). The resulting reaction was stirred at room temperature for 16 h. H₂O (30 mL) was added to the reaction, extracted with EtOAc (30 mL×3). The combined organic layers were washed with brine, dried over Na₂SO₄, concentrated in vacuo to give the crude, which was purified with Prep-TLC (DMC/MeOH=10/1) to give RAGE 293 (30 mg, yield: 27%) as a white solid. LC-MS [M+H]⁺: 374.1, Rt=0.920 min. Purity: 100 (214 nm), 99.88 (254 nm). ¹H NMR (400 MHz, CDCl₃) δ 9.39 (d, J=4.0 Hz, 1H), 8.68-8.66 (m, 1H), 8.57 (s, 1H), 8.42-8.38 (m, 2H), 8.08 (s, 1H), 7.91 (s, 1H), 7.75-7.73 (m, 1H), 5.68 (s, 1H), 4.01 (s, 2H), 3.76-3.74 (m, 4H), 2.58-2.54 (m, 4H).

### Example 8: Synthesis of Analog RAGE 294 According to the Invention

Compound RAGE 294 according to the present disclosure was prepared as shown in Scheme 8 and described below.

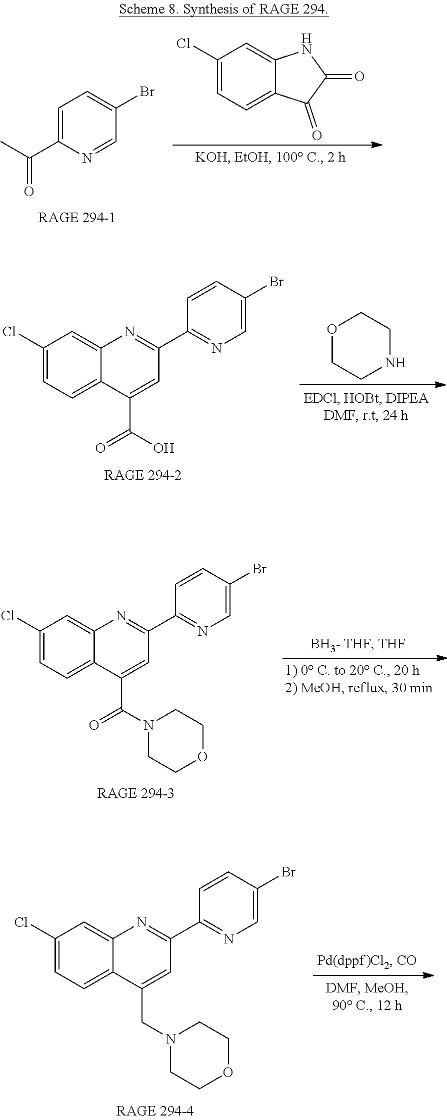

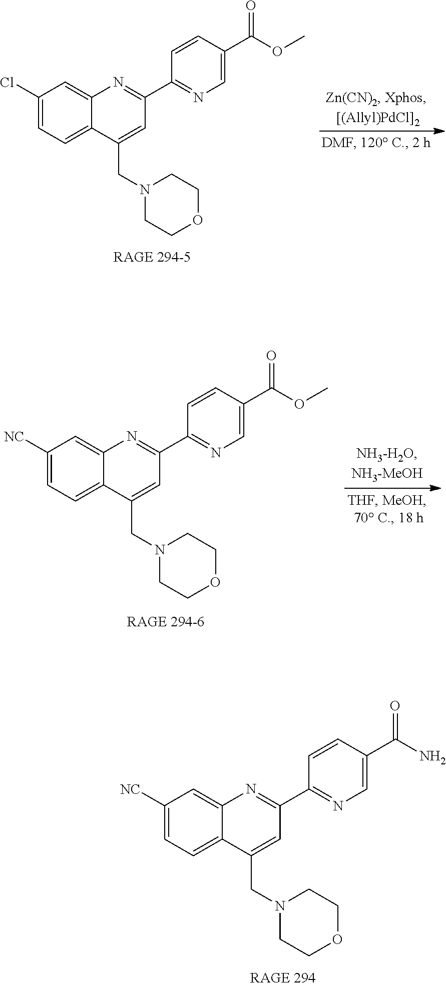 [SMILES: COC(=O)c1ccc(-c2cc(CN3CCOCC3)c3ccc(Cl)cc3n2)nc1 ; COC(=O)c1ccc(-c2cc(CN3CCOCC3)c3ccc(C#N)cc3n2)nc1 ; N#Cc1ccc2c(CN3CCOCC3)cc(-c3ccc(C(N)=O)cn3)nc2c1]

### Example 8A: Preparation of Compound RAGE 294-2

To a solution of 1-(5-bromopyridin-2-yl)ethan-1-one (46.4 g, 232.1 mmol) in EtOH (450 mL) was added a solution of 6-chloroindoline-2,3-dione (35 g, 193.4 mmol) in 6M aqueous KOH (150 mL). The mixture was stirred at 100° C. for 2 hours. After the reaction was completed, the reaction was concentrated in vacuo to remove EtOH, the residue was dissolved in water (350 mL). The pH of the solution was adjusted to 3 by HCl (4 N). The yellow solid was formed. The mixture was filtered, washed with water (200 mL) and DCM (250 mL). The collected filtered cake was dried to give the RAGE 294-2 (70 g crude) as a yellow solid, which was used into the next step without further purification. LC-MS [M+H]⁺: 363.1.

### Example 8B: Preparation of Compound RAGE 294-3

To a solution of 2-(5-bromopyridin-2-yl)-7-chloroquinoline-4-carboxylic acid (70 g crude) in DMF (350 mL) was added morpholine (42.1 g, 483.5 mmol), HOBT (39.1 g, 289.5 mmol), EDCI (55.3 g, 289.5 mmol) and DIPEA (74.7 g, 579 mmol). The resulting reaction was stirred at room temperature for 24 hours. After the reaction was completed, the reaction was diluted with brine (400 mL), extracted with EtOAc (400 mL×3). The combined organic layers were washed with brine (500 mL), dried over Na₂SO₄, concentrated in vacuo to give the crude product, which was purified by silica gel column chromatography (petroleum ether/ethyl acetate, 2:1) to provide RAGE 294-3 (45 g, yield: 53.9%) as a yellow solid. LC-MS [M+H]⁺: 432.2.

### Example 8C: Preparation of Compound RAGE 294-4

To a solution of (2-(5-bromopyridin-2-yl)-7-chloroquinolin-4-yl)(morpholino)methanone (39 g, 90.3 mmol) in THF (225 mL) was added BH₃-THF (225 mL, 1 M solution in THF, 225 mmol) at 0° C. slowly. The resulting reaction was stirred at room temperature for 20 hours. After the reaction was completed, the reaction was quenched with MeOH (80 mL), and then refluxed for another 30 min. The resulting solution was concentrated in vacuo to give the crude, which was purified by silica gel column chromatography (DCM/MeOH, 15:1) to provide RAGE 294-4 (25 g, yield: 66.1%) as a yellow solid. LC-MS [M+H]⁺: 418.2.

### Example 8D: Preparation of Compound RAGE 294-5

A mixture of 4-((2-(5-bromopyridin-2-yl)-7-chloroquinolin-4-yl)methyl)morpholine (20 g, 47.8 mmol), Pd(dppf)Cl₂ (3.46 g, 4.78 mmol) and TEA (14.5 g, 143.4 mmol) in DMF/MeOH (50 mL/50 mL) was stirred at 90° C. under CO atmosphere for 12 hours. After the reaction was completed, the reaction was diluted with water (100 mL), extracted with EtOAc (100 mL×3). The combined organic layers were washed with brine (100 mL), dried over Na₂SO₄, concentrated in vacuo to give the crude product which was purified by silica gel column chromatography (petroleum ether/ethyl acetate, 3:1) to provide RAGE 294-5 (6.5 g, yield: 34.2%) as a white solid. LC-MS [M+H]⁺: 398.2

### Example 8E: Preparation of Compound RAGE 294-6

A mixture of methyl 6-(7-chloro-4-(morpholinomethyl)quinolin-2-yl) nicotinate (1 g, 2.52 mmol) Zn(CN)₂ (589.7 mg, 5.04 mmol), Xphos (238 mg, 0.5 mmol) and [Pd(Allyl)Cl]₂ (183 mg, 0.5 mmol) in DMF (7 mL) was degassed for 10 mins, and heated with microwave at 120° C. for 2 hours. After the reaction was completed, the reaction cooled to room temperature, diluted with H₂O (40 mL), extracted with EtOAc (50 mL×3). The combined organic layers were washed with brine (80 mL), dried over Na₂SO₄, concentrated in vacuo to give the crude, which was purified by silica gel column chromatography (DCM/MeOH, 20:1) to provide RAGE 294-6 (450 mg, yield: 46%) as a yellow solid. LC-MS [M+H]⁺: 389.2.

### Example 8F: Preparation of Compound RAGE 294

A solution of methyl 6-(7-cyano-4-(morpholinomethyl)quinolin-2-yl) nicotinate (167 mg, 0.43 mmol) and NH₄OH (10 mL) in THF (15 mL)/MeOH (7 M, NH₃ solution in MeOH, 10 mL) was sealed in tube and stirred at 70° C. for 18 hours. After the reaction was completed, the reaction was concentrated in vacuo to give the crude product, which was purified by silica Prep-HPLC to provide RAGE 294 (30 mg, yield: 18.7%) as a white solid. LC-MS [M+H]⁺: 374.2. LC-MS [M+H]⁺: 374.1, Rt=0.959 min. Purity: 98.35 (214 nm), 96.41 (254 nm). ¹H NMR (400 MHz, CDCl3) δ 9.12 (s, 1H), 8.61-8.59 (m, 2H), 8.42-8.40 (m, 2H), 8.20-8.27 (m, 1H), 7.62 (s, 1H), 7.52 (s, 1H), 6.29 (s, 1H), 3.88 (s, 2H), 3.58-3.55 (m, 4H), 2.46-2.45 (m, 4H).

### Example 9: Synthesis of Analog RAGE 295 According to the Invention

Compound RAGE 295 according to the present disclosure was prepared as shown in Scheme 9 and described below.

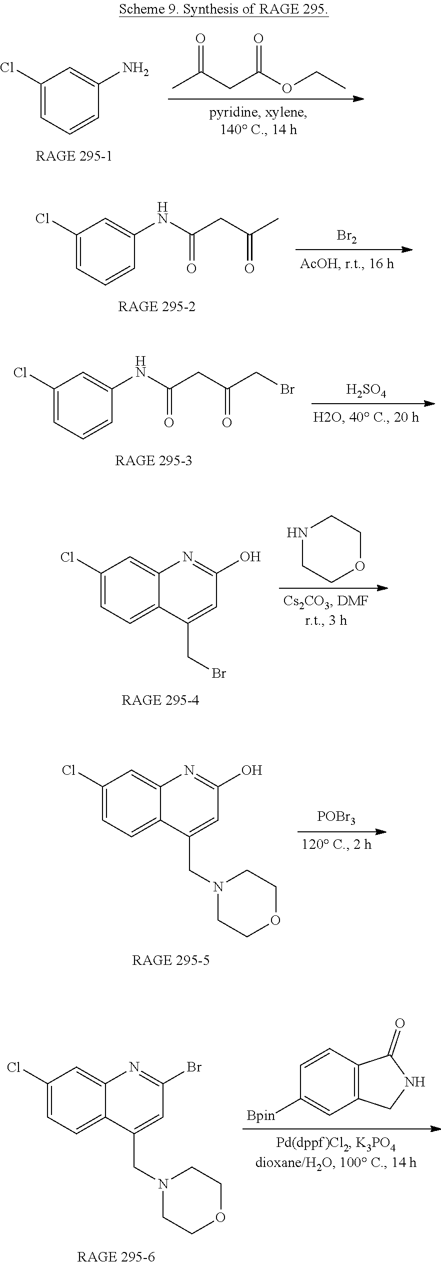

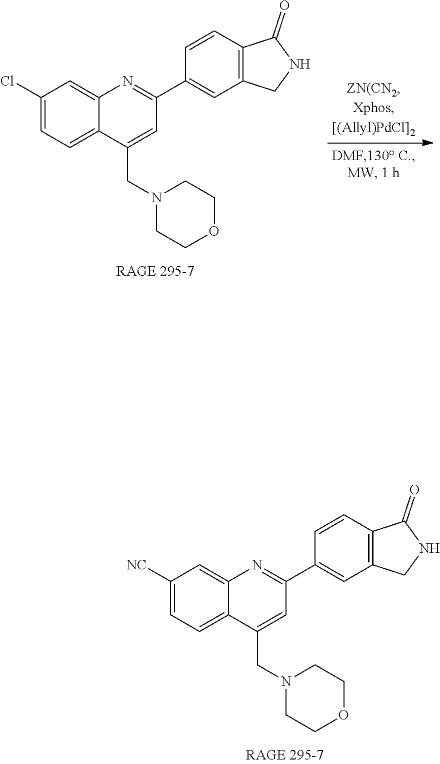 [SMILES: O=C1NCc2cc(-c3cc(CN4CCOCC4)c4ccc(Cl)cc4n3)ccc21 ; N#Cc1ccc2c(CN3CCOCC3)cc(-c3ccc4c(c3)CNC4=O)nc2c1]

### Example 9A: Preparation of Compound RAGE 295-2

To a solution of 3-chloroaniline (10 g, 78.7 mmol) in xylene (200 mL) was added ethyl 3-oxobutanoate (20.5 g, 157.4 mmol) and pyridine (12.4 g, 157.4 mmol). The resulting mixture was stirred at 140° C. for 14 h. After the reaction was completed, the reaction was quenched with saturated aqueous H₂O (300 mL), extracted with EtOAc (150 mL×3). The combined organic layers were washed with brine (40 mL), dried over Na₂SO₄, concentrated in vacuo to give the crude product which was purified by silica gel column chromatography (petroleum ether/ethyl acetate, 5:1) to provide RAGE 295-2 (5 g, yield: 30.0%) as a yellow solid. LC-MS [M+H]⁺: 212.2.

### Example 9B: Preparation of Compound RAGE 295-3

To a solution of N-(3-chlorophenyl)-3-oxobutanamide (5 g, 23.6 mmol) in AcOH (80 mL) was added Br₂ (5.7 g, 35.4 mmol) dropwise at 0° C. The resulting mixture was stirred at room temperature for 16 h. After the reaction was completed, the reaction mixture was evaporated and the pH of the residue was adjusted with NaHCO₃ (sat. aq.) to 8, extracted with EtOAc (50 mL×3) and concentrated in vacuo to give the crude, which was purified with silica gel chromatography (petroleum ether/ethyl acetate, 5/1) to give desired RAGE 295-3 (6 g, yield: 87.7%) as a yellow solid. LC-MS [M+H]⁺: 290.2.

### Example 9C: Preparation of Compound RAGE 295-4

To a solution of 4-bromo-N-(3-chlorophenyl)-3-oxobutanamide (6 g, 20.8 mmol) in H₂O (100 mL) was added H₂SO₄ (20.4 g, 208 mmol) dropwise at 0° C. The resulting mixture was stirred at 40° C. for 20 h. After the reaction was completed, the reaction was quenched with saturated aqueous NaHCO₃ (300 mL), extracted with EtOAc (100 mL×3). The combined organic layers were washed with brine (100 mL), dried over Na₂SO₄, concentrated in vacuo to give the crude, which was purified with silica gel chromatography (petroleum ether/ethyl acetate, 1/1) to give desired RAGE 295-4 (4 g, yield: 70.7%) as a yellow solid. LC-MS [M+H]⁺: 272.2.

### Example 9D: Preparation of Compound RAGE 295-5

To a solution of 4-(bromomethyl)-7-chloroquinolin-2-ol (4 g, 14.8 mmol) in DMF (60 mL) was added morpholine (1.9 g, 22.2 mmol) and Cs₂CO₃ (9.6 g, 29.6 mmol). The resulting mixture was stirred at room temperature for 3 h. After the reaction was completed, the reaction was quenched with H₂O (150 mL), extracted with EtOAc (100 mL×3). The combined organic layers were washed with brine (100 mL), dried over Na₂SO₄, concentrated in vacuo to give the crude product which was purified by silica gel column chromatography (petroleum ether/ethyl acetate, 2:1) to provide RAGE 295-5 (3 g, yield: 72.6%) as a yellow solid. LC-MS [M+H]⁺: 279.2.

### Example 9E: Preparation of Compound RAGE 295-6

A solution of 7-chloro-4-(morpholinomethyl)quinolin-2-ol (1 g, 3.6 mmol) and POBr₃ (5 g) was stirred at 120° C. for 2 h. The organic layer was evaporated, and the pH of the aqueous phase was adjusted with saturated aqueous NaHCO₃ to 8, and then extracted with EtOAc (30 mL×3). The combined organic layers were washed with brine (30 mL), dried over Na₂SO₄, concentrated in vacuo to give desired RAGE 295-6 (650 mg, yield: 52.9%)) as a yellow solid. LC-MS [M+H]⁺: 341.2.

### Example 9F: Preparation of Compound RAGE 295-7

To a solution of 4-((2-bromo-7-chloroquinolin-4-yl)methyl)morpholine (400 mg, 1.2 mmol) in dioxane/H₂O (10 ml/1 ml) was added Pd(dppf)Cl₂ (88 mg, 0.12 mmol), 5-(4,4,5,5-tetramethyl-1,3,2-dioxaborolan-2-yl) isoindolin-1-one (466 mg, 1.8 mmol) and K₃PO₄ (763 mg, 3.6 mmol). The reaction was stirred at 140° C. for 14 h under nitrogen, then was quenched with H₂O (20 mL), extracted with EtOAc (20 mL×3). The combined organic layers were washed with brine (30 mL), dried over Na₂SO₄, concentrated in vacuo to give the crude, which was purified with silica gel chromatography (petroleum ether/ethyl acetate, 1/1) to give desired RAGE 295-7 (230 mg, yield: 48.6%) as a yellow solid. LC-MS [M+H]⁺: 394.2.

### Example 9G: Preparation of Compound RAGE 295

To a solution of 5-(7-chloro-4-(morpholinomethyl)quinolin-2-yl) isoindolin-1-one (200 mg, 0.5 mmol), Zn(CN)₂ (176 mg, 1.5 mmol), Xphos (24 mg, 0.05 mmol) and [Pd(allyl)Cl]₂ (18 mg, 0.05 mmol) in DMF (3 mL) was stirred at 130° C. by microwave for 1 h. The mixture reaction was filtered and washed with EtOAc (10 mL×3). The filtrate was concentrated in vacuo to give the crude, which was purified silica gel chromatography (DCM/MeOH=10/1) to give desired RAGE 295 (10 mg, yield: 5.2%) as a yellow solid. LC-MS [M+H]⁺: 385.2. LC-MS [M+H]⁺: 385.2, Rt=0.922 min. Purity: 98.17 (214 nm), 97.72 (254 nm). ¹H NMR (400 MHz, DMSO-d6) δ 8.72 (s, 1H), 8.61 (s, 1H), 8.50 (d, J=8.0 Hz, 1H), 8.46 (s, 1H), 8.38 (d, J=8.0 Hz, 1H), 8.32 (s, 1H), 7.92 (d, J=8.0 Hz, 1H), 7.83 (d, J=8.0 Hz, 1H), 4.49 (s, 2H), 4.01 (s, 2H), 3.54-3.45 (m, 4H), 2.55-2.51 (m, 4H).

### Example 10: Synthesis of Analog RAGE 296 According to the Invention

Compound RAGE 296 according to the present disclosure was prepared as shown in Scheme 10 and described below.

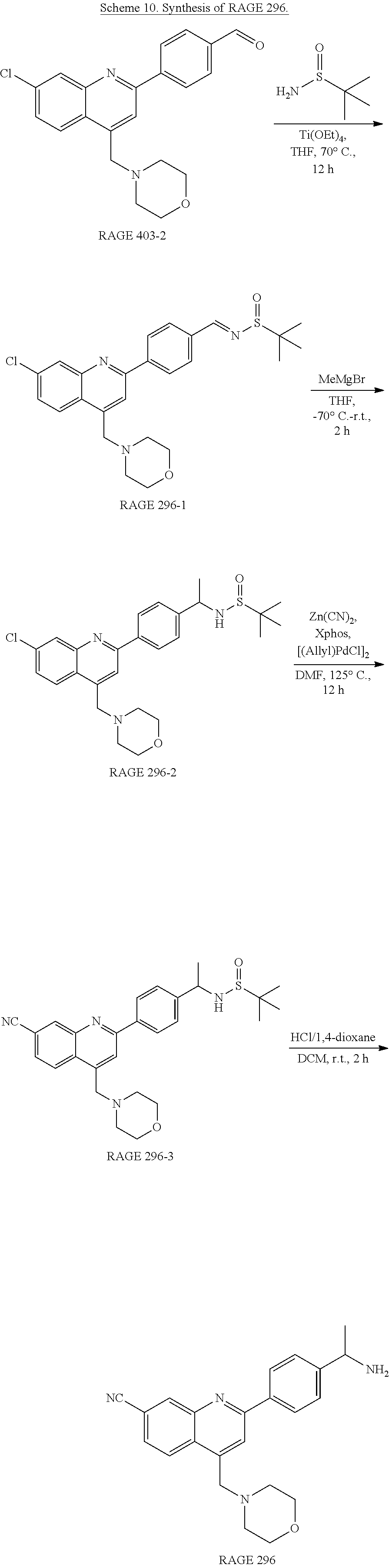

### Example 10A: Preparation of Compound RAGE 296-1

To a solution of 4-(7-chloro-4-(morpholinomethyl)quinolin-2-yl)benzaldehyde (1 g, 2.7 mmol) in THF (20 mL) was added 2-methylpropane-2-sulfinamide (496 mg, 4.1 mmol) and Ti(OEt)₄ (935 mg, 4.1 mmol). The resulting mixture was stirred at 70° C. for 12 h. After the reaction was completed, the reaction was quenched with saturated aqueous NH₄Cl (30 mL), extracted with EtOAc (40 mL×3). The combined organic layers were washed with brine (40 mL), dried over Na₂SO₄, concentrated in vacuo to give the crude product, which was purified by silica gel column chromatography (petroleum ether/ethyl acetate, 2:1) to provide RAGE 296-1 (1.1 g, yield: 86.7%) as a yellow solid. LC-MS [M+H]⁺: 470.2.

### Example 10B: Preparation of Compound RAGE 296-2

To a solution of (E)-N-(4-(7-chloro-4-(morpholinomethyl)quinolin-2-yl)benzylidene)-2-methylpropane-2-sulfinamide (1.1 g, 2.3 mmol) in THF (20 mL) was added MeMgBr (4.6 mL, 4.6 mmol) at −78° C. under nitrogen. The resulting mixture was stirred at room temperature for 2 h. After the reaction was completed, the reaction was quenched with saturated aqueous NH₄Cl (30 mL), extracted with EtOAc (20 mL×3). The combined organic layers were washed with brine (20 mL), dried over Na₂SO₄, concentrated in vacuo to give the crude product, which was purified by silica gel column chromatography (petroleum ether/ethyl acetate, 2:1) to provide RAGE 296-2 (1 g, yield: 89.5%) as a yellow solid. LC-MS [M+H]⁺: 486.2.

### Example 10C: Preparation of Compound RAGE 296-3

To a solution of (2-(6-bromopyridin-3-yl)-7-chloroquinolin-4-yl) (morpholino)methanone (1 g, 2.1 mmol), Zn(CN)₂ (731 mg, 6.3 mmol), Xphos (95 mg, 0.2 mmol) and [Pd(allyl)Cl]₂ (73 mg, 0.2 mmol) in DMF (20 mL) was stirred at 125° C. for 2 h. The mixture reaction was filtered and washed with EtOAc (20 mL×3). The filtrate was concentrated in vacuo to give the crude, which was purified silica gel chromatography (DCM/MeOH=10/1) to give RAGE 296-3 (500 mg, yield: 49.9%) as a yellow solid. LC-MS [M+H]⁺: 477.2.

### Example 10D: Preparation of Compound RAGE 296

A solution of N-(1-(4-(7-cyano-4-(morpholinomethyl)quinolin-2-yl)phenyl)ethyl)-2-methylpropane-2-sulfinamide (150 mg, 0.3 mmol) and HCl (2 mL, 4 M in dioxane, 8 mmol) in DCM (4 mL) was stirred at room temperature for 2 h. The organic layer was evaporated, and the residue was washed with saturated aqueous NaHCO₃ (15 mL), extracted with EtOAc (15 mL×3). The combined organic layers were washed with brine (25 mL), dried over Na₂SO₄, and concentrated to provide the desired RAGE 296 (14 mg, yield: 12.5%)) as a white solid. LC-MS [M+H]⁺: 373.2, Rt=0.866 min. Purity: 99.05 (214 nm), 99.59 (254 nm). ¹H NMR (400 MHz, CDCl3) δ 8.51 (s, 1H), 8.37 (d, J=12 Hz, 1H), 8.15 (d, J=12 Hz, 2H), 7.99 (s, 1H), 7.68-7.65 (m, 1H), 7.57-7.55 (m, 2H), 4.29-4.24 (m, 1H), 3.96 (s, 2H), 3.74-3.72 (m, 4H), 1.48 (d, J=4 Hz, 3H).

### Example 11: Synthesis of Analog RAGE 297 According to the Invention

Compound RAGE 297 according to the present disclosure was prepared as shown in Scheme 11 and described below.

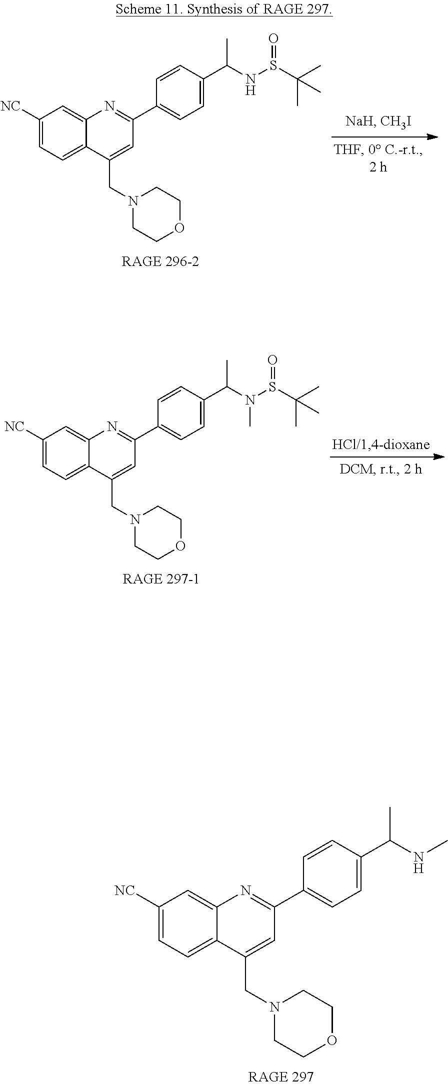 [SMILES: CC(NS(=O)C(C)(C)C)c1ccc(-c2cc(CN3CCOCC3)c3ccc(C#N)cc3n2)cc1 ; CC(c1ccc(-c2cc(CN3CCOCC3)c3ccc(C#N)cc3n2)cc1)N(C)S(=O)C(C)(C)C ; CNC(C)c1ccc(-c2cc(CN3CCOCC3)c3ccc(C#N)cc3n2)cc1]

### Example 11A: Preparation of Compound RAGE 297-1

To a solution of N-(1-(4-(7-cyano-4-(morpholinomethyl)quinolin-2-yl)phenyl)ethyl)-2-methylpropane-2-sulfinamide (400 mg, 0.8 mmol) in THF (10 mL) was added NaH (48 mg, 1.2 mmol, 60% suspension in paraffin oil) at 0° C. The mixture was stirred at 0° C. for 0.5 h. Then CH₃I (170 mg, 1.2 mmol) was added at 0° C. The resulting mixture was stirred at rt for 2 h. After the reaction was completed, the reaction was quenched with saturated aqueous NH₄Cl (15 mL), extracted with EtOAc (10 mL×3). The combined organic layers were washed with brine (20 mL), dried over Na₂SO₄, concentrated in vacuo to give the crude product which was purified by silica gel column chromatography (petroleum ether/ethyl acetate, 2:1) to provide RAGE 297-1 (300 mg, yield: 76.4%) as a white solid. LC-MS [M+H]⁺: 491.2.

### Example 11B: Preparation of Compound RAGE 297

A solution of 2-(4-(1-(methylamino)ethyl)phenyl)-4-(morpholinomethyl)quinoline-7-carbonitrile (300 mg, 0.6 mmol) and HCl (5 mL, 4 M in dioxane, 20 mmol) in DCM (10 mL) was stirred at room temperature for 2 h. The reaction was concentrated in vacuo, saturated aqueous NaHCO₃ (20 mL) was added and extracted with EtOAc (20 mL×3). The combined organic layers were washed with brine (30 mL), dried over Na₂SO₄ and concentrated in vacuo to give the crude product, which was purified by Prep-HPLC to provide the desired RAGE 297 (150 mg, yield: 64.6%)) as a white solid. LC-MS [M+H]⁺: 387.2, Rt=0.874 min. Purity: 95.74 (214 nm), 95.10 (254 nm). ¹H NMR (400 MHz, DMSO-d6) δ 8.58 (d, J=4 Hz, 1H), 8.50 (d, J=8 Hz, 1H), 8.25-8.23 (m, 3H), 7.92-7.89 (m, 1H), 7.53 (d, J=8 Hz, 2H), 4.03 (s, 2H), 3.69-3.66 (m, 1H), 3.58-3.56 (m, 4H), 2.55-2.53 (m, 4H), 2.17 (s, 3H), 1.29 (d, J=8 Hz, 3H).

### Example 12: Synthesis of Analog RAGE 298 According to the Invention

Compound RAGE 298 according to the present disclosure was prepared as shown in Scheme 12 and described below.

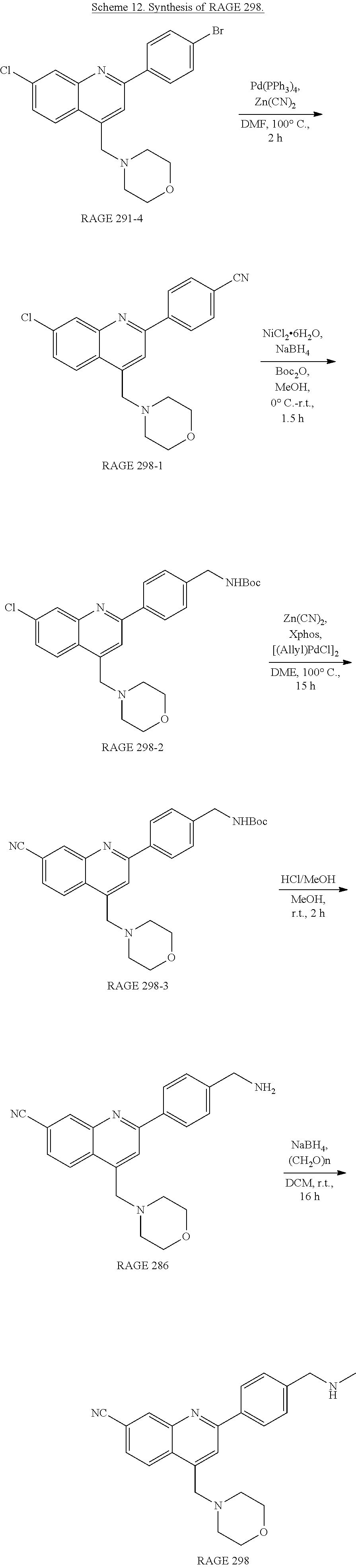 [SMILES: Clc1ccc2c(CN3CCOCC3)cc(-c3ccc(Br)cc3)nc2c1 ; N#Cc1ccc(-c2cc(CN3CCOCC3)c3ccc(Cl)cc3n2)cc1 ; CC(C)(C)OC(=O)NCc1ccc(-c2cc(CN3CCOCC3)c3ccc(Cl)cc3n2)cc1 ; CC(C)(C)OC(=O)NCc1ccc(-c2cc(CN3CCOCC3)c3ccc(C#N)cc3n2)cc1 ; N#Cc1ccc2c(CN3CCOCC3)cc(-c3ccc(CN)cc3)nc2c1 ; CNCc1ccc(-c2cc(CN3CCOCC3)c3ccc(C#N)cc3n2)cc1]

### Example 12A: Preparation of Compound RAGE 298-1

To a solution of 4-((2-(4-bromophenyl)-7-chloroquinolin-4-yl)methyl)morpholine 1 (2 g, 4.78 mmol) in DMF (20 mL) was added ZnCN₂ (297 mg, 2.53 mmol) and Pd(PPh₃)₄ (552 mg, 0.478 mmol). The resulting mixture was stirred at 100° C. for 2 h under N₂ atmosphere. After the reaction was completed, the reaction was filtered, and then diluted with H₂O (50 mL), extracted with EtOAc (40 mL×3). The combined organic layers were washed with brine (40 mL), dried over Na₂SO₄, concentrated in vacuo to give the crude product, which was purified by silica gel column chromatography (petroleum ether/ethyl acetate, 1:1) to provide RAGE 298-1 (1.1 g, yield: 63.2%) as a white solid. LC-MS [M+H]⁺: 364.1.

### Example 12B: Preparation of Compound RAGE 298-2

To a solution of 4-(7-chloro-4-(morpholinomethyl)quinolin-2-yl)benzonitrile 2 (1.1 g, 3.02 mmol) in dry MeOH (20 mL) was added Boc₂O (1.32 g, 6.04 mmol) and NiCl₂·6H₂O (71.8 mg, 0.30 mmol) at 0° C. Then NaBH₄ (800 mg, 21.14 mmol) was added in small portions over 30 min. The resulting reaction mixture was allowed to warm to room temperature and left to stir for a further 1 h. The mixture was concentrated and the residue was dissolved in EtOAc (100 mL) and washed with saturated NaHCO₃ (2×50 mL). The organic layer was dried over Na₂SO₄, and the solvent removed in vacuo provide RAGE 298-2 (850 mg, yield: 60.2%) as a pale-yellow solid. LC-MS [M+H]⁺: 468.1.

### Example 12C: Preparation of Compound RAGE 298-3

To a solution of tert-butyl (4-(7-chloro-4-(morpholinomethyl)quinolin-2-yl)benzyl)carbamate 3 (850 mg, 1.82 mmol) in DME (10 mL) was added Zn(CN)₂ (425 mg, 3.64 mmol), allylpalladium (II) chloride dimer (66.6 mg, 0.18 mmol) and X-phos (173.5 mg, 0.36 mmol). The resulting mixture was stirred at 100° C. for 15 h under N₂ atmosphere. After the reaction was completed, the reaction was filtered, and then diluted with H₂O (50 mL), extracted with EtOAc (40 mL×3). The combined organic layers were washed with brine (40 mL), dried over Na₂SO₄, concentrated in vacuo to give the crude product, which was purified by silica gel column chromatography (petroleum ether/ethyl acetate, 1:1) to provide RAGE 298-3 (500 mg, yield: 59.9%) as a pale-yellow solid. LC-MS [M+H]⁺: 459.2.

### Example 12D: Preparation of Compound RAGE 286

To a solution of tert-butyl (4-(7-cyano-4-(morpholinomethyl)quinolin-2-yl)benzyl)carbamate (500 mg, 1.09 mmol) in dry DCM (8 mL) was added 4 mol/L HCl-1,4-dioxane (1.36 mL, 5.45 mmol). The reaction was stirred at room temperature for 3 h. The reaction was concentrated in vacuo, and then neutralization with 7 mol/L NH₃/MeOH solution till pH=8. After concentrated in vacuo, the mixture was purified by silica gel column chromatography (DCM/MeOH, 10:1) to provide RAGE 286 (250 mg, yield: 64%) as a pale-yellow solid. LC-MS [M+H]⁺: 359.2.

### Example 12E: Preparation of Compound RAGE 298

To a solution of 2-(4-(aminomethyl)phenyl)-4-(morpholinomethyl)quinoline-7-carbonitrile (100 mg, 0.28 mmol) in anhydrous CH₂Cl₂ (10 mL) was added paraformaldehyde (25 mg, 0.84 mmol). The reaction was stirred at room temperature for 16 hours. The reaction was cooled to −10° C. and sodium borohydride (42 mg, 1.12 mmol) was added. Anhydrous methanol (0.5 mL) was added dropwise and the reaction was allowed to warm to room temperature with stirring for another 3 hours. The reaction mixture was concentrated under vacuum. The residue was purified by Prep-HPLC to give the desired RAGE 298 (50 mg, yield: 36.7%, TFA salt) as a white solid. LC-MS [M+H]⁺: 373.3, Rt=0.874 min. Purity: 99.40 (214 nm), 99.48 (254 nm). ¹H NMR (400 MHz, MeOD-d4) δ 8.50 (s, 1H), 8.47-8.45 (m, 2H), 8.42 (d, J=8 Hz, 2H), 7.94 (d, J=12 Hz, 1H), 7.70 (d, J=12 Hz, 2H), 4.91 (s, 2H), 4.30 (s, 2H), 3.78 (s, 4H), 3.30-3.29 (m, 4H), 2.77 (s, 3H).

### Example 13: Synthesis of Analog RAGE 299 According to the Invention

Compound RAGE 299 according to the present disclosure was prepared as shown in Scheme 13 and described below.

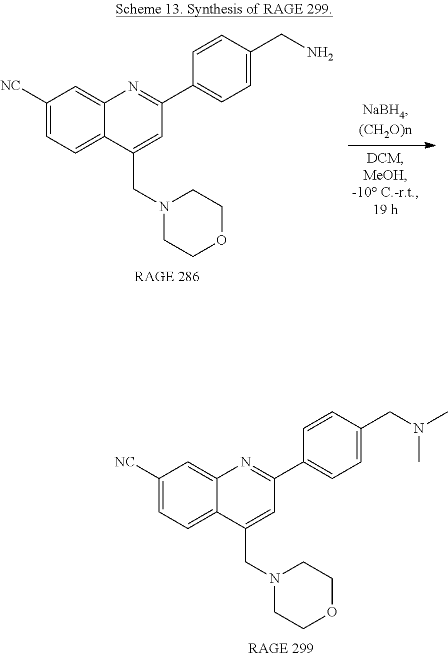 [SMILES: N#Cc1ccc2c(CN3CCOCC3)cc(-c3ccc(CN)cc3)nc2c1 ; CN(C)Cc1ccc(-c2cc(CN3CCOCC3)c3ccc(C#N)cc3n2)cc1]

### Example 13A: Preparation of Compound RAGE 299

To a solution of 2-(4-(aminomethyl)phenyl)-4-(morpholinomethyl)quinoline-7-carbonitrile 5 (250 mg, 0.70 mmol) in anhydrous DCM (8 mL) was added paraformaldehyde (210 mg, 7.0 mmol). The reaction was refluxed for 16 hours. The reaction was cooled to −10° C. and sodium borohydride (132.4 mg, 3.5 mmol) was added. Anhydrous methanol (1 mL) was added dropwise and the reaction was allowed to warm to room temperature with stirring over 3 hour. The reaction mixture was concentrated under vacuum. The residue was purified by Prep-HPLC to give the desired RAGE 299 (25 mg, yield: 8.2%, FA salt) as a white solid. LC-MS [M+H]⁺: 388.1, Rt=1.001 min. Purity: 100 (214 nm), 100 (254 nm). ¹H NMR (400 MHz, MeOD-d4) δ 8.56 (d, J=8.0 Hz, 1H), 8.52 (d, J=1.2 Hz, 1H), 8.33 (d, J=8.0 Hz, 2H), 8.21 (s, 1H), 7.82-7.78 (m, 1H), 7.65 (d, J=8.2 Hz, 2H), 4.16 (s, 2H), 4.07 (s, 2H), 3.71-3.68 (m, 4H), 2.72 (s, 6H), 2.59-2.57 (m, 4H).

### Example 14: Synthesis of Analog RAGE 400 According to the Invention

Compound RAGE 400 according to the present disclosure was prepared as shown in Scheme 14 and described below.

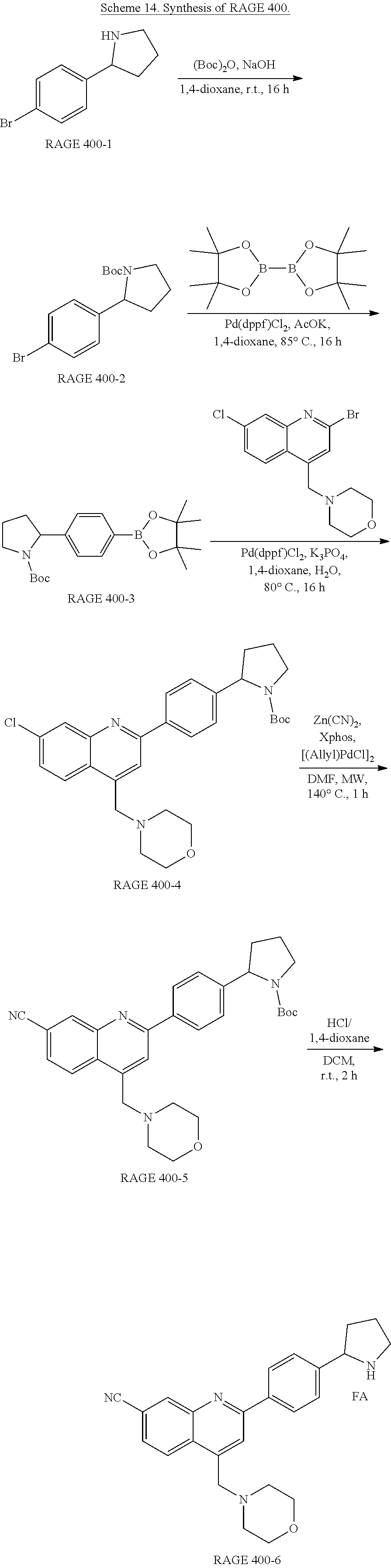

### Example 14A: Preparation of Compound RAGE 400-2

To Boc₂O (1.38 mg, 6.30 mmol) was added to a stirred solution of 2-(4-bromophenyl)pyrrolidine 1 (950 mg, 4.20 mmol) and 2M NaOH aqueous solution (4.2 mL, 8.4 mmol) in 1,4-dioxane (10 mL) at 0° C. The reaction mixture was stirred at room temperature for 16 h. H₂O (30 mL) was added and extracted with EtOAc (30 mL×3). The combined organic phases were washed with brine (30 mL), dried over Na₂SO₄, and concentrated in vacuo. The residue was purified by column chromatography on silica gel (petroleum ether/ethyl acetate, 10:1) to provide RAGE 400-2 (1.40 g, yield: quant.) as colorless sticky oil. LC-MS [M+H]⁺: 327.2.

### Example 14B: Preparation of Compound RAGE 400-3

A solution of tert-butyl 2-(4-bromophenyl)pyrrolidine-1-carboxylate (1.0 g, 3.07 mmol), 4,4,4′,4′,5,5,5′,5′-octamethyl-2,2′-bi (1,3,2-dioxaborolane) (2.3 g, 9.20 mmol), Pd(dppf)Cl₂ (449 mg, 0.61 mmol), and KOAc (602 mg, 6.13 mmol) in 1,4-dioxane (15 mL) was stirred at 85° C. for 16 h under N₂ atmosphere. The mixture was cooled to room temperature and filtered by celite. The filtrate was concentrated in vacuo to give crude product, which was purified by column chromatography on silica gel (petroleum ether/ethyl acetate, 10:1) to give RAGE 400-3 (750 mg, yield: 65%) as pale-yellow sticky oil. LC-MS [M+H]⁺: 374.2.

### Example 14C: Preparation of Compound RAGE 400-4

To a solution of 4-((2-bromo-7-chloroquinolin-4-yl)methyl)morpholine (200 mg, 0.59 mmol) and tert-butyl 2-(4-(4,4,5,5-tetramethyl-1,3,2-dioxaborolan-2-yl)phenyl)pyrrolidine-1-carboxylate (1,3,2-dioxaborolane) 3 (438 mg, 1.17 mmol) in 1,4-dioxane (15 mL) and H₂O (2 mL) was added Pd(dppf)Cl₂ (86 mg, 0.12 mmol) and K₃PO₄ (249 mg, 1.17 mmol). The mixture was stirred at 80° C. for 16 h under N₂ atmosphere. The mixture was cooled to room temperature and filtered by celite. The filtrate was concentrated in vacuo to give crude product, which was purified by column chromatography on silica gel (DCM/MeOH, 10:1) to give RAGE 400-4 (150 mg, yield: 50%) as a white solid. LC-MS [M+H]⁺: 508.1.

### Example 14D: Preparation of Compound RAGE 400-5

To a solution of tert-butyl 2-(4-(7-chloro-4-(morpholinomethyl)quinolin-2-yl)phenyl)pyrrolidine-1-carboxylate (100 mg, 0.20 mmol) and Zn(CN)₂ (47 mg, 0.40 mmol) in DMF (2 mL) was added allylpalladium (II) chloride dimer (7 mg, 0.02 mmol) and X-phos (19 mg, 0.04 mmol). The mixture was stirred in a sealed tube. After degassing with N₂ for 1 min, the reaction mixture was irradiated in microwave at 140° C. for 1 h. The mixture was cooled to room temperature, diluted with H₂O (20 mL), and then extracted with EtOAc (30 mL×3). The combined organics was washed with brine (50 mL), dried over Na₂SO₄, and concentrated in vacuo. The residue was purified by column chromatography on silica gel (petroleum ether/ethyl acetate, 2:1) to give RAGE 400-5 (80 mg, yield: 82%) as a white solid. LC-MS [M+H]⁺: 499.2

### Example 14E: Preparation of Compound RAGE 400

HCl (4 N in 1,4-dioxane, 1 mL) was added to a solution of tert-butyl 2-(4-(7-cyano-4-(morpholinomethyl)quinolin-2-yl)phenyl)pyrrolidine-1-carboxylate 6 (80 mg, 0.16 mol) in DCM (2 mL). After stirred at room temperature for 2 h, the mixture concentrated and neutralized with NaHCO₃ (sat. aq.) till the pH=8˜9. The reaction was concentrated in vacuo to give the crude product, which was purified by Prep-HPLC to provide the desired RAGE 400 (20 mg, yield: 28%, FA salt) as a white solid. LC-MS [M+H]⁺: 399.2, Rt=0.954 min. Purity: 97.99 (214 nm), 98.70 (254 nm). ¹H NMR (400 MHz, DMSO-d6) δ 8.59 (s, 1H), 8.55 (d, J=8 Hz, 1H), 8.49-8.26 (m, 4H), 7.90 (d, J=8 Hz, 1H), 7.62 (d, J=8 Hz, 2H), 4.33-4.32 (m, 1H), 4.03 (s, 2H), 3.59-3.56 (m, 4H), 3.19-3.16 (m, 1H), 3.11-3.09 (m, 1H), 2.49-2.43 (m, 4H), 2.26-2.23 (m, 1H), 1.90-1.89 (m, 2H), 1.75-1.73 (m, 1H).

### Example 15: Synthesis of Analog RAGE 401 According to the Invention

Compound RAGE 401 according to the present disclosure was prepared as shown in Scheme 15 and described below.

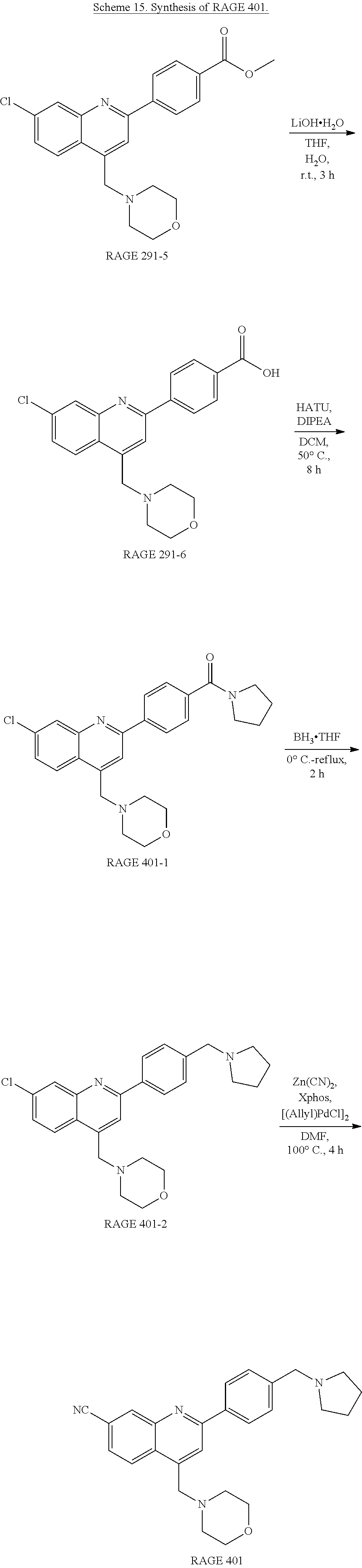 [SMILES: COC(=O)c1ccc(-c2cc(CN3CCOCC3)c3ccc(Cl)cc3n2)cc1 ; "LiOH·H2O" ; O=C(O)c1ccc(-c2cc(CN3CCOCC3)c3ccc(Cl)cc3n2)cc1 ; O=C(c1ccc(-c2cc(CN3CCOCC3)c3ccc(Cl)cc3n2)cc1)N1CCCC1 ; Clc1ccc2c(CN3CCOCC3)cc(-c3ccc(CN4CCCC4)cc3)nc2c1 ; N#Cc1ccc2c(CN3CCOCC3)cc(-c3ccc(CN4CCCC4)cc3)nc2c1]

### Example 15A: Preparation of Compound RAGE 291-6

To a solution of methyl 4-(7-chloro-4-(morpholinomethyl)quinolin-2-yl)benzoate 1 (1 g, 2.52 mmol) in THF/H₂O (10 mL/3 mL) was added LiOH·H₂O (159 mg, 3.78 mmol). The resulting mixture was stirred at room temperature for 3 h. After the reaction was completed, the reaction mixture was acidified with 1M HCl till pH=4. The mixture was diluted with water (20 mL), and then extracted with EtOAc (30 mL). The combined organic phase was washed with brine (20 mL), dried over Na₂SO₄, filtered, and concentrated in vacuo to provide RAGE 291-6 (800 mg, yield: 83%) as a white solid. LC-MS [M+H]⁺: 383.1.

### Example 15B: Preparation of Compound RAGE 401-1

A solution of 4-(7-chloro-4-(morpholinomethyl)quinolin-2-yl)benzoic acid 2 (800 mg, 2.09 mmol), DIPEA (540 mg, 4.18 mmol), HATU (1192 mg, 3.13 mmol) and pyrrolidine (223 mg, 3.13 mmol) in DCM (10 mL) was stirred at 50° C. for 8 h. The mixture was cooled to room temperature. The resulting mixture was diluted with H₂O (20 mL) and extracted with EtOAc (25 mL×3). The combined organic layers were washed with brine (20 mL), dried over Na₂SO₄, and concentrated in vacuo to give the crude product, which was purified by silica gel column chromatography (petroleum ether/ethyl acetate, 2:1) to provide RAGE 401-1 (750 mg, yield: 82%) as a white solid. LC-MS [M+H]⁺: 436.2

### Example 15C: Preparation of Compound RAGE 401-2

BH₃·THF (5.2 mL, 1M in THF, 5.2 mmol) was added dropwise to a stirred solution of (4-(7-chloro-4-(morpholinomethyl)quinolin-2-yl)phenyl) (pyrrolidin-1-yl)methanone 3 (750 mg, 1.72 mmol) in THF (10 mL) at 0° C. The mixture was stirred at 65° C. for 2 h. The reaction mixture was cooled to room temperature and quenched with MeOH (20 mL). The mixture was concentrated in vacuo to give the crude product, which was purified by silica gel column chromatography (petroleum ether/ethyl acetate, 2:1) to provide RAGE 401-2 (300 mg, yield: 41%) as a white solid. LC-MS [M+H]⁺: 422.2.

### Example 15D: Preparation of Compound RAGE 401

A solution of 4-((7-chloro-2-(4-(pyrrolidin-1-ylmethyl)phenyl)quinolin-4-yl)methyl)morpholine 4 (300 mg, 0.71 mmol), Zn(CN)₂ (167 mg, 1.42 mmol), allylpalladium (II) chloride dimer (26 mg, 0.07 mmol), and Xphos (68 mg, 0.14 mmol) in DMF (5 mL) was stirred in a sealed tube. After degassing with N₂ for 1 min, the reaction mixture was stirred at 100° C. for 4 h. The mixture was cooled to room temperature, diluted with H₂O (20 mL), and then extracted with EtOAc (30 mL×3). The combined organics was washed with brine (50 mL), dried over Na₂SO₄, and concentrated in vacuo to give the crude product which was purified by Prep-HPLC to provide the desired RAGE 401 (33 mg, yield: 8.8%, TFA salt) as a white solid. LC-MS [M+H]⁺: 413.3, Rt=0.905 min. Purity: 100 (214 nm), 99.90 (254 nm). ¹H NMR (400 MHz, MeOD-d4) δ 8.65 (s, 1H), 8.53-8.48 (m, 2H), 8.43 (d, J=8.2 Hz, 2H), 7.95-7.92 (m, 1H), 7.74 (d, J=8.0 Hz, 2H), 4.50 (s, 2H), 3.88 (s, 4H), 3.53 (s, 2H), 3.32-3.32 (m, 4H), 3.31-3.27 (m, 4H), 2.15-2.11 (m, 2H), 2.04-2.02 (m, 2H).

### Example 16: Synthesis of Analog RAGE 402 According to the Invention

Compound RAGE 402 according to the present disclosure was prepared as shown in Scheme 16 and described below.

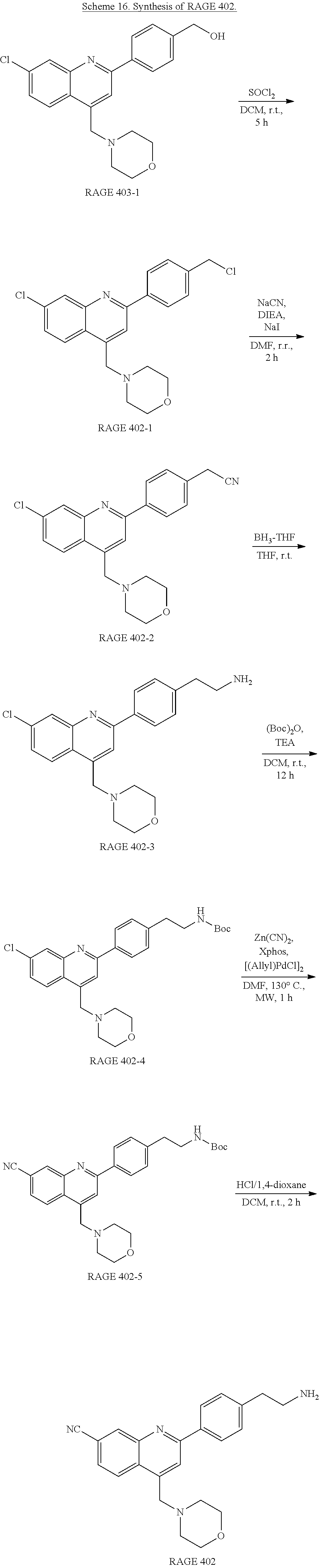

### Example 16A: Preparation of Compound RAGE 402-1

To a solution of (4-(7-chloro-4-(morpholinomethyl)quinolin-2-yl)phenyl) methanol (1 g, 2.71 mmol) in dry DCM (15 mL) was added SOCl₂ (390 mL, 5.42 mmol). The resulting mixture was stirred at room temperature for 5 h under N₂ atmosphere. The reaction was concentrated in vacuo. The residue was purified by silica gel column chromatography (petroleum ether/ethyl acetate, 3:1) to provide RAGE 402-1 (900 mg, yield: 85.7%) as a white solid. LC-MS [M+H]⁺: 387.1.

### Example 16B: Preparation of Compound RAGE 402-2

To a solution of 4-((7-chloro-2-(4-(chloromethyl)phenyl)quinolin-4-yl)methyl)morpholine (900 mg, 2.32 mmol) in dry DMF (10 mL) was added DIPEA (899 mg, 6.97 mmol), NaCN (227 mg, 4.64 mmol) and NaI (34.8 mg, 0.23 mmol). The reaction was stirred at room temperature for 2 h. The reaction was diluted with H₂O (50 mL), and then extracted with EtOAc (40 mL×3). The combined organic phase was washed with brine (50 mL), dried over Na₂SO₄, filtered and concentrated in vacuo. The residue was purified by silica gel column chromatography (petroleum ether/ethyl acetate, 3:1) to provide 402-2 (710 mg, yield: 81%) as a white solid. LC-MS [M+H]⁺: 378.1.

### Example 16C: Preparation of Compound RAGE 402-3

To a solution of 2-(4-(7-chloro-4-(morpholinomethyl)quinolin-2-yl)phenyl) acetonitrile (200 mg, 0.53 mmol) in dry THF (5 mL) was added 1 mol/L BH₃·THF (2.65 mL, 2.65 mmol) dropwise over 15 min while stirring under nitrogen atmosphere. After refluxing for 1 hour, the mixture was then cooled to 0° C., and MeOH was added to quench the reaction. The resulting mixture was stirred under reflux for 1 h then concentrated to provide RAGE 402-3 (200 mg, crude) as a pale yellow solid. LC-MS [M+H]⁺: 382.1.

### Example 16D: Preparation of Compound RAGE 402-4

To a solution of 2-(4-(7-chloro-4-(morpholinomethyl)quinolin-2-yl)phenyl)ethan-1-amine (200 mg, 0.52 mmol) in DCM (5 mL) was added TEA (157 mg, 1.56 mmol) and (Boc) 20 (170 mg, 0.78 mmol). The mixture was stirred at room temperature for 12 h. The reaction was concentrated in vacuo. The residue was purified by silica gel column chromatography (petroleum ether/ethyl acetate, 1:2) to provide RAGE 402-4 (200 mg, yield: 79.8%) as a white solid. LC-MS [M+H]⁺: 482.1.

### Example 16E: Preparation of Compound RAGE 402-5

To a solution of tert-butyl (4-(7-chloro-4-(morpholinomethyl)quinolin-2-yl) phenethyl)carbamate (200 mg, 0.41 mmol) in dry DMF (5 mL) was added Zn(CN)₂ (53 mg, 0.46 mmol), allylpalladium (II) chloride dimer (15 mg, 0.04 mmol) and X-phos (38 mg, 0.08 mmol). The resulting mixture was stirred at 130° C. for 1 h with microwave under N₂ atmosphere. After the reaction was completed, the reaction was filtered, and then diluted with H₂O (20 mL), extracted with DCM (30 mL×3). The combined organic layers were washed with brine (20 mL), dried over Na₂SO₄, concentrated in vacuo to give the crude product, which was purified by silica gel column chromatography (petroleum ether/ethyl acetate, 1:2) to provide RAGE 402-5 (80 mg, yield: 41.3%) as a pale-yellow solid. LC-MS [M+H]⁺: 473.2.

### Example 16F: Preparation of Compound RAGE 402

To a solution of tert-butyl (4-(7-cyano-4-(morpholinomethyl)quinolin-2-yl) phenethyl)carbamate (80 mg, 0.17 mmol) in dry DCM (2 mL) was added 4 mol/L HCl/1,4-dioxane (0.42 mL, 1.7 mmol). After stirring at room temperature for 2 h, the reaction was concentrated in vacuo, and then neutralized with 7 mol/L NH₃/MeOH solution till pH=8. The mixture was concentrated in vacuo, and then purified by Prep-HPLC to provide the desired RAGE 402 (18.2 mg, yield: 28.7%) as a white solid. LC-MS [M+H]⁺: 373.2, Rt=0.824 min. Purity: 97.11 (214 nm), 97.03 (254 nm). ¹H NMR (400 MHz, DMSO-d6) δ 8.59 (s, 1H), 8.50 (d, J=8.0 Hz, 1H), 8.33 (s, 2H), 8.27-8.24 (m, 3H), 7.90 (d, J=4.2 Hz, 1H), 7.46 (d, J=8.4 Hz, 2H), 4.03 (s, 2H), 3.57 (d, J=4.2 Hz, 4H), 2.89 (d, J=4.2 Hz, 2H), 2.67 (s, 2H), 2.51-2.49 (m, 4H).

### Example 17: Synthesis of Analog RAGE 403 According to the Invention

Compound RAGE 403 according to the present disclosure was prepared as shown in Scheme 17 and described below.

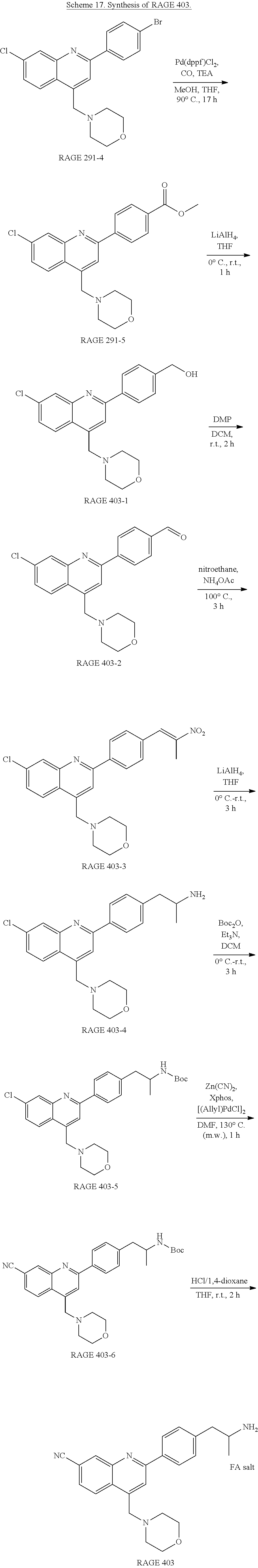

### Example 17A: Preparation of Compound RAGE 291-5

A mixture of 4-((2-(4-bromophenyl)-7-chloroquinolin-4-yl)methyl)morpholine (2 g, 4.81 mmol), Pd(dppf)Cl₂ (352 mg, 0.48 mmol) and TEA (2.42 g, 24 mmol) in MeOH/THF (25 mL/25 mL) was stirred at 90° C. under CO atmosphere for 17 hours. After the reaction was completed, the mixture reaction was filtered through Celite, washed with EtOAc (100 mL). The filtrate was concentrated in vacuo to give the crude, which was purified with silica gel choreography (petroleum ether/ethyl acetate, 1:1) to provide RAGE 291-5 (1.3 g, yield: 68.1%) as a yellow solid. LC-MS [M+H]⁺: 397.2.

### Example 17B: Preparation of Compound RAGE 403-1

To a solution of methyl 4-(7-chloro-4-(morpholinomethyl)quinolin-2-yl)benzoate (1.5 g, 3.78 mmol) in dry THF (50 mL) was added LiAlH₄ (5.67 mL, 1 M solution in THF, 5.67 mmol) at ° C. The resulting reaction was stirred at room temperature for 1 hour. After the reaction was completed, the reaction was quenched with Na₂SO₄-10 H₂O (10 g) at 0° C. The mixture was filtered, washed with EtOAc (50 mL). The filtrate was concentrated in vacuo to give the crude, which was purified with silica gel choreography (DCM/MeOH, 15:1) to give RAGE 403-1 (1.2 g, yield: 86.3%) as a yellow solid. LC-MS [M+H]⁺: 369.2.

### Example 17C: Preparation of Compound RAGE 403-2

To a solution of (4-(7-chloro-4-(morpholinomethyl)quinolin-2-yl)phenyl) methanol (1.2 g, 3.25 mmol) in DCM (35 mL) was added Dess-Martin Periodinane reagent (2.1 g, 4.87 mmol) at 0° C. The resulting reaction was stirred at room temperature for 2 hours. After the reaction was completed, the reaction was quenched with saturated NaHCO3 (50 mL), extracted with DCM (50 mL×3). The combined organic layers were was washed with brine (100 mL). The organic layers was dried over Na2SO4, concentrated in vacuo to give the crude, which was purified with silica gel choreography (DCM/MeOH, 25:1) to give RAGE 403-2 (1.08 g, yield: 90.8%) as a white solid. LC-MS [M+H]⁺: 367.1.

### Example 17D: Preparation of Compound RAGE 403-3

A mixture (E)-4-((7-chloro-2-(4-(2-nitroprop-1-en-1-yl)phenyl)quinolin-4-yl)methyl)morpholine (864 mg, 2.36 mmol) and NH4OAc (1.82 g, 23.6 mmol) in nitroethane (5 mL) was stirred at 100° C. for 3 hours. After the reaction was completed, the reaction was diluted with H₂O (50 mL), extracted with EtOAc (50 mL×3). The combined organic layers were washed with brine (100 mL), dried over Na2SO4, concentrated in vacuo to give the crude, which was purified with ilica gel choreography (DCM/MeOH, 20:1) to give RAGE 403-3 (750 mg, yield: 75.1%) as a yellow solid. LC-MS [M+H]⁺: 424.2.

### Example 17E: Preparation of Compound RAGE 403-4

To a solution of (E)-4-((7-chloro-2-(4-(2-nitroprop-1-en-1-yl)phenyl)quinolin-4-yl)methyl)morpholine (740 mg, 1.75 mmol) in THF (25 mL) was added LiAlH₄ (2.6 mL, 1 M solution in THF, 2.6 mmol) at 0° C. The resulting reaction was stirred at room temperature for 3 hours. After the reaction was completed, the reaction was quenched with Na₂SO₄-10 H₂O (5 g). The mixture reaction was filtered, washed with EtOAc (50 mL). The filtrate was concentrated in vacuo to give RAGE 403-4 (1 g, crude) as a yellow solid, which was used into the next step without further purification. LC-MS [M+H]⁺: 396.2.

### Example 17F: Preparation of Compound RAGE 403-5

To a solution of 1-(4-(7-chloro-4-(morpholinomethyl)quinolin-2-yl)phenyl)propan-2-amine (1 g, crude from previous step) and TEA (353.5 mg, 3.5 mmol) in DCM (30 mL) was added Boc₂O (567 mg, 2.6 mmol) at 0° C. The resulting reaction was stirred at room temperature for 3 hours. After the reaction was completed, the reaction was quenched with H₂O (50 mL), extracted with DCM (50 mL×3). The combined organic layers were washed with brine (70 mL), dried over Na2SO4, concentrated in vacuo to give crude, which was purified with silica gel chromatography (DCM/MeOH, 30:1) to provide RAGE 403-5 (240 mg, over 2 step yield: 27.6%) as a white solid. LC-MS [M+H]⁺: 496.2.

### Example 17G: Preparation of Compound RAGE 403-6

A mixture of tert-butyl (1-(4-(7-chloro-4-(morpholinomethyl)quinolin-2-yl)phenyl)propan-2-yl)carbamate (240 mg, 0.48 mmol), Zn(CN)₂ (112.3 mg, 0.96 mmol), Xphos (90.4 mg, 0.19 mmol) and [Pd(Allyl)Cl]₂ (35 mg, 0.096 mmol) in DMF (4 mL) was degassed for 10 mins, and heated with microwave at 130° C. for 1 hour. After the reaction was completed, the reaction cooled to room temperature, diluted with H₂O (30 mL), extracted with EtOAc (30 mL×3). The combined organic layers were washed with brine (60 mL), dried over Na₂SO₄, concentrated in vacuo to give the crude, which was purified Prep-TLC (DCM/MeOH, 20:1) to provide RAGE 403-6 (170 mg, yield: 72.9%) as a white solid. LC-MS [M+H]⁺: 487.2.

### Example 17E: Preparation of Compound RAGE 403

To a solution of tert-butyl (1-(4-(7-cyano-4-(morpholinomethyl)quinolin-2-yl)phenyl)propan-2-yl)carbamate (170 mg, 0.35 mmol) in dioxane (5 mL) was added HCl (0.7 mL, 4 M solution in 1,4-dioxane, 2.8 mmol). The reaction was stirred at room temperature for 2 hours. After the reaction was completed, the reaction was concentrated in vacuo to give the crude, which was purified with Prep-HPLC to provide the desired RAGE 403 (40 mg, yield: 29.6%) as a white solid. LC-MS [M+H]⁺: 387.1, Rt=0.892 min. Purity: 100 (214 nm), 100 (254 nm). ¹H NMR (400 MHz, DMSO-d6) δ 8.56 (s, 1H), 8.47 (d, J=8.8 Hz, 1H), 8.37 (s, 1H), 8.25-8.20 (m, 3H), 7.88 (d, J=8.9 Hz, 1H), 7.41 (d, J=8.9 Hz, 2H), 4.00 (s, 2H), 3.54 (s, 4H), 3.32-3.30 (m, 1H), 2.94-2.91 (m, 1H), 2.70-2.65 (m, 1H), 2.47 (s, 4H), 1.06 (d, J=8.9 Hz, 3H).

### Example 18: Synthesis of Analog RAGE 404 According to the Invention

Compound RAGE 404 according to the present disclosure was prepared as shown in Scheme 18 and described below.

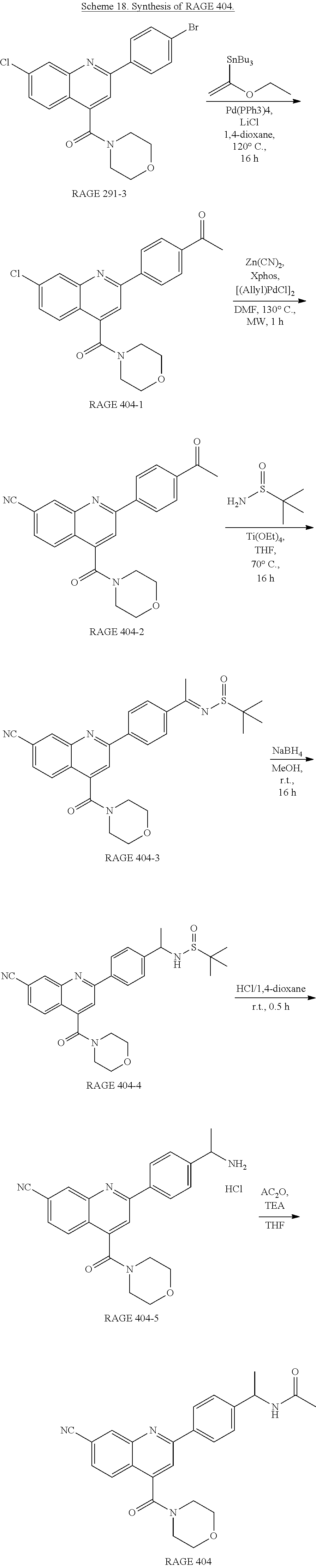

### Example 18A: Preparation of Compound RAGE 404-1

A mixture of (2-(4-bromophenyl)-7-chloroquinolin-4-yl)(morpholino)methanone (5 g, 11.6 mmol), tributyl(1-ethoxyvinyl) stannane (8.38 g, 23.2 mmol), LiCl (0.97 g, 23.2 mmol), Pd(PPh₃)₄ (1.34 g, 1.16 mmol) in 1,4-dioxane (100 mL) was stirred at 120° C. for 16 hours under N₂ atmosphere. After the reaction was completed, the reaction was filtered through celite, washed with EtOAc (100 mL×2). The filtrate was concentrated in vacuo to give the crude, which was purified by silica gel column chromatography (petroleum ether/ethyl acetate, 2:1) to provide RAGE 404-1 (4.25 g, yield: 92.8%) as a yellow solid. LC-MS [M+H]⁺: 395.2.

### Example 18B: Preparation of Compound RAGE 404-2

A mixture of 1-(4-(7-chloro-4-(morpholine-4-carbonyl)quinolin-2-yl)phenyl)ethan-1-one (2.3 g, 5.8 mmol), Zn(CN)₂ (1.36 g, 11.6 mmol), Xphos (552.2 mg, 1.16 mmol) and [Pd(Allyl)Cl]₂ (212 mg, 0.58 mmol) in DMF (15 mL) was degassed by N₂ for 10 min, then sealed and heated with microwave at 130° C. for 1 hour. After the reaction was completed, the reaction was cooled to room temperature, diluted with H₂O (50 mL), extracted with EtOAc (60 mL×3). The combined organic layers were washed with brine (60 mL), dried over Na₂SO₄, concentrated in vacuo to give the crude, which was purified with silica gel chromatography (petroleum ether/ethyl acetated, 2/1) to provide 404-2 (1.1 g, yield: 49.3%) as a yellow solid. LC-MS [M+H]⁺: 386.2

### Example 18C: Preparation of Compound RAGE 404-3

A solution of 2-(4-acetylphenyl)-4-(morpholine-4-carbonyl)quinoline-7-carbonitrile (1.1 g, 2.85 mmol), 2-methylpropane-2-sulfinamide (517.9 mg, 4.28 mmol) and Ti(OEt)₄ (1.3 g, 5.7 mmol) in THF (30 mL) was stirred at 70° C. for 16 hours under N₂ atmosphere. After the reaction was completed, the reaction was quenched with water (50 mL), extracted with EtOAc (60 mL×3). The combined organic layers was washed with brine (80 mL), dried over Na₂SO₄, concentrated in vacuo to give the crude, which was purified by silica gel chromatography (petroleum ether/ethyl acetated, 3/1) to provide RAGE 404-3 (1 g, yield: 71.9%) as a yellow solid. LC-MS [M+H]⁺: 489.2.

### Example 18D: Preparation of Compound RAGE 404-4

To a solution of (E)-N-(1-(4-(7-cyano-4-(morpholine-4-carbonyl)quinolin-2-yl)phenyl)ethylidene)-2-methylpropane-2-sulfinamide (500 mg, 1.02 mmol) in MeOH (15 mL) was added NaBH₄ (76 mg, 2 mmol). The resulting reaction mixture was stirred at room temperature for 16 hours. After the reaction was completed, the reaction was quenched with saturated aqueous NH₄Cl (30 mL), extracted with EtOAc (40 mL×3). The combined organic layers were washed with brine (50 mL), dried over Na₂SO₄, concentrated in vacuo to give the crude, which was purified with silica gel chromatography (petroleum ether/ethyl acetated, 1/1) to provide RAGE 404-4 (320 mg, 64%) as a yellow solid. LC-MS [M+H]⁺: 491.2.

### Example 18E: Preparation of Compound RAGE 404-5

To a solution of N-(1-(4-(7-cyano-4-(morpholine-4-carbonyl)quinolin-2-yl)phenyl)ethyl)-2-methylpropane-2-sulfinamide (320 mg, 0.65 mmol) in 1,4-dioxnae (5 mL) was added HCl (1.6 mL, 4 M solution in 1,4-dioxnae, 6.4 mmol). The resulting reaction was stirred at room temperature for 30 mins. After the reaction was completed, the reaction was concentrated in vacuo to provide the desired RAGE 404-5 as HCl salt (350 mg crude), which was used into next step without further purification. LC-MS [M+H]⁺: 387.2.

### Example 18F: Preparation of Compound RAGE 404

2-(4-(1-aminoethyl)phenyl)-4-(morpholine-4-carbonyl)quinoline-7-carbonitrile (350 mg crude) and TEA (262.2 mg, 2.6 mmol) in THF (10 mL) was added acetic anhydride (132.6 mg, 1.3 mmol). The reaction was stirred at room temperature for 3 hour. After the reaction was completed, the reaction quenched with water (25 mL), extracted with EtOAc (30 mL×3). The combined organic layers were washed with brine (50 mL), dried over Na₂SO₄, concentrated in vacuo to give the crude, which was purified Prep-HPLC to provide the desired RAGE 404 (35 mg, yield over 2 steps: 12.6%) as a white solid. LC-MS [M+H]⁺: 429.2, Rt=1.147 min. Purity: 99.23 (214 nm), 98.19 (254 nm). ¹H NMR (400 MHz, DMSO-d6) δ 8.67 (s, 1H), 8.39 (d, J=8.0 Hz, 1H), 8.30 (s, 1H), 8.27 (d, J=8.0 Hz, 2H), 8.00 (d, J=8.0 Hz, 1H), 7.91 (d, J=8.0 Hz, 1H), 7.52 (d, J=12.0 Hz, 2H), 5.01-4.98 (m, 1H), 3.88-3.85 (s, 1H), 3.81-3.75 (m, 3H), 3.65-3.52 (m, 1H), 3.40-3.36 (m, 1H), 3.25-3.21 (m, 1H), 3.18-3.12 (m, 1H), 1.88 (s, 3H), 1.39 (d, J=8.0 Hz, 3H).

### Example 19: Synthesis of Analog RAGE 405 According to the Invention

Compound RAGE 405 according to the present disclosure was prepared as shown in Scheme 19 and described below.

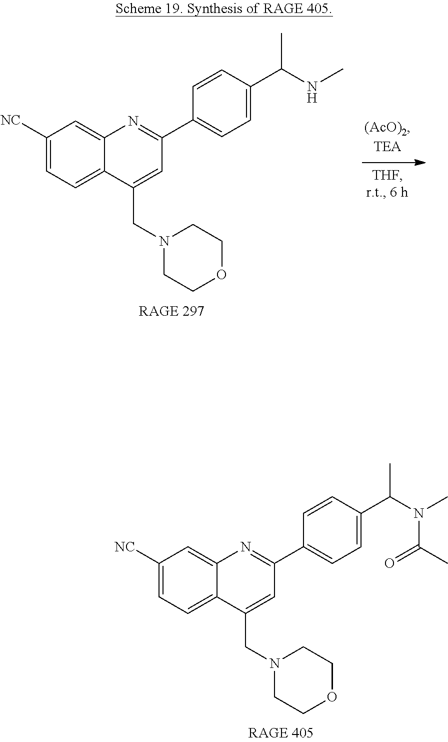 [SMILES: CNC(C)c1ccc(-c2cc(CN3CCOCC3)c3ccc(C#N)cc3n2)cc1 ; CC(=O)N(C)C(C)c1ccc(-c2cc(CN3CCOCC3)c3ccc(C#N)cc3n2)cc1]

### Example 19A: Preparation of Compound RAGE 405

To a solution of 2-(4-(1-(methylamino)ethyl)phenyl)-4-(morpholinomethyl)quinoline-7-carbonitrile (150 mg, 0.4 mmol) and TEA (121 mg, 1.2 mmol) in THF (10 mL) was added Ac2O (71 mg, 0.6 mmol) at 0° C. The mixture was stirred at room temperature for 6 h. The reaction was washed with water (25 mL) and extracted with EtOAc (20 mL×3). The combined organic layers were washed with brine (30 mL), dried over Na₂SO₄ and concentrated in vacuo to give the crude product, which was purified by silica gel column chromatography (DCM/MeOH, 20:1) to provide the desired RAGE 405 (35 mg, yield: 20.4%)) as a white solid. ESI-MS [M+H]⁺: 429.2. LC-MS [M+H]⁺: 429.3, RT=0.996 min. Purity: 97.79 (214 nm), 97.61 (254 nm). 1H NMR (400 MHz, DMSO-d6) δ 8.60 (s, 1H), 8.50 (d, J=8 Hz, 1H), 8.31-8.25 (m, 3H), 7.92 (d, J=12 Hz, 1H), 7.51-7.45 (m, 2H), 5.93-5.88 (m, 1H), 4.03 (s, 2H), 3.59-3.56 (m, 4H), 2.71 (s, 2H), 2.58 (s, 1H), 2.51-2.48 (m, 4H) 2.19 (s, 1H), 2.08 (s, 2H), 1.60 (d, J=8 Hz, 1H), 1.48 (d, J=8 Hz, 2H).

### Example 20: Synthesis of Analog RAGE 406 According to the Invention

Compound RAGE 406 according to the present disclosure was prepared as shown in Scheme 20 and described below.

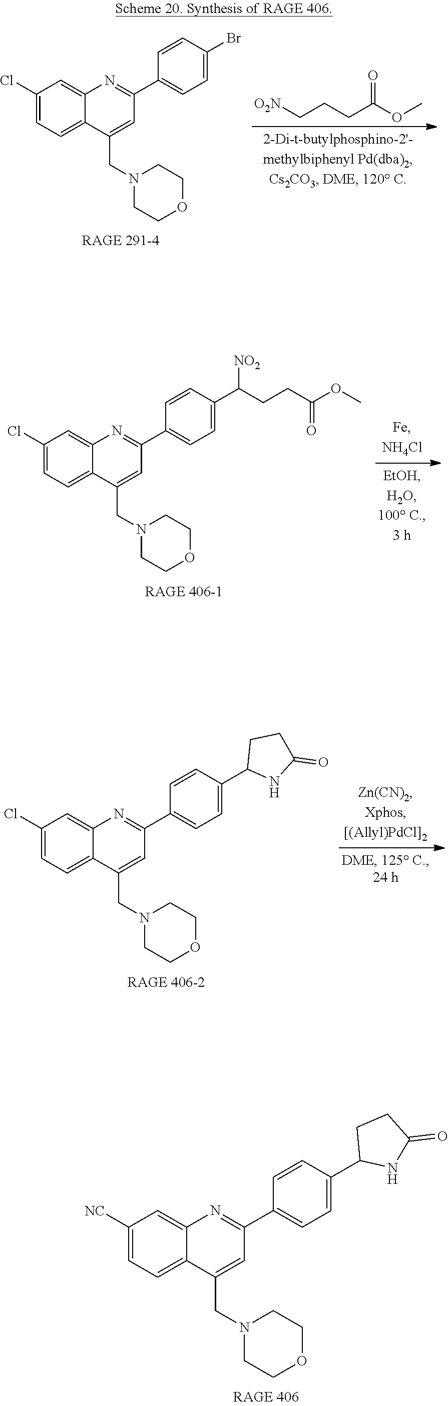

### Example 20A: Preparation of Compound RAGE 406-1

A mixture of 4-((2-(4-bromophenyl)-7-chloroquinolin-4-yl)methyl)morpholine (2 g, 4.8 mmol), methyl 4-nitrobutanoate (1.41 g, 9.6 mmol), 2-Di-t-butylphosphino-2′-methylbiphenyl (299.5 mg, 0.96 mmol), Pd2(dba)3 (351.4 mg, 0.48 mmol) and Cs2CO3 (3.91 g, 12 mmol) in DME (50 mL) was stirred at 120° C. for 12 hours. After the reaction was completed, the reaction was diluted with water (50 mL), extracted with EtOAc (60 mL×3). The combine organic layers were washed with brine (60 mL), dried over Na2SO4, concentrated in vacuo to give the crude, which was purified by silica gel column chromatography (DCM/MeOH, 15:1) to provide RAGE 406-1 (800 mg, yield: 34.5%) as a yellow solid. LC-MS [M+H]⁺: 484.2.

### Example 20B: Preparation of Compound RAGE 406-2

A mixture of methyl 4-(4-(7-chloro-4-(morpholinomethyl)quinolin-2-yl)phenyl)-4-nitrobutanoate (800 mg, 1.65 mmol), Fe (924 mg, 16.5 mmol) and NH₄Cl (1.75 g, 33 mmol) in EtOH/water (5 mL/5 mL) was stirred at 100° C. for 3 hours. After the reaction was completed, the reaction mixture was filtered, washed with MeOH (30 mL). The filtrate was concentrated in vacuo to give the crude, which was purified by silica gel column chromatography (DCM/MeOH, 10:1) to provide RAGE 406-2 (170 mg, yield: 24.5%) as a yellow solid. LC-MS [M+H]⁺: 422.2.

### Example 20C: Preparation of Compound RAGE 406

A mixture of 5-(4-(7-chloro-4-(morpholinomethyl)quinolin-2-yl)phenyl) pyrrolidin-2-one (170 mg, 0.4 mmol), Zn(CN)₂ (93.6 mg, 0.8 mmol), Xphos (38.1 mg, 0.08 mmol) and [Pd(Allyl)Cl]₂ (14.6 mg, 0.04 mmol) in DME (5 mL) was stirred at 120° C. for 24 hours. After the reaction was completed, the reaction was cooled to room temperature, diluted with H2O (20 mL), extracted with EtOAc (30 mL×3). The combined organic layers were washed with brine (40 mL), dried over Na₂SO₄, concentrated in vacuo to give the crude, which was purified by Prep-HPLC to provide RAGE 406 (20 mg, yield: 12.1%) as a white solid. LC-MS [M+H]⁺: 413.2, Rt=1.013 min. Purity: 96.19 (214 nm), 96.12 (254 nm). ¹H NMR (400 MHz, DMSO-d6) δ 8.60 (s, 1H), 8.50 (d, J=8.0 Hz, 1H), 8.30-8.28 (m, 3H), 8.19 (s, 1H), 7.93-7.90 (m, 1H), 7.51 (d, J=8.0 Hz, 2H), 4.78 (t, J=8.0 Hz, 1H), 4.03 (s, 2H), 3.56 (t, J=4.0 Hz, 4H), 2.62-2.40 (m, 5H), 2.28 (t, J=8.0 Hz, 2H), 1.86-1.74 (m, 1H).

### Example 21: Synthesis of Analog RAGE 407 According to the Invention

Compound RAGE 407 according to the present disclosure was prepared as shown in Scheme 21 and described below.

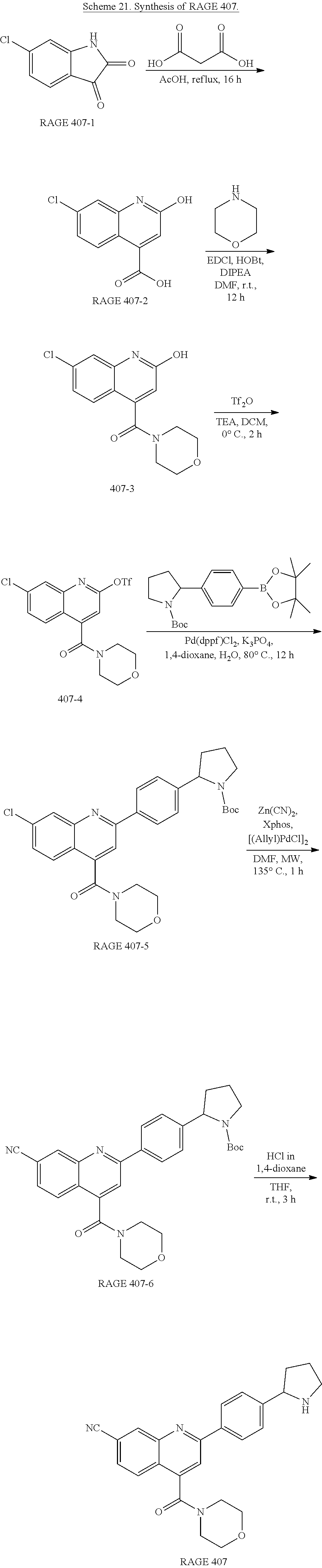

### Example 21A: Preparation of Compound RAGE 407-2

6-chloroindoline-2,3-dione (10 g, 55.2 mmol) and malonic acid (22.9 g, 220.8 mmol) in AcOH (150 mL) was stirred 100° C. for 16 hours. After the reaction was completed, the reaction was concentrated in vacuo to give the crude, which was triturated by petroleum ether/ethyl acetate (5:1, 100 mL) to provide RAGE 407-2 (8.5 g, yield: 69.1%) as a yellow solid. LC-MS [M+H]⁺: 224.1.

### Example 21B: Preparation of Compound RAGE 407-3

7-chloro-2-hydroxyquinoline-4-carboxylic acid (8.5 g, 37.95 mmol), morpholine (6.6 g, 75.9 mmol), HOBT (7.69 g, 56.93 mmol), EDCI (10.87 g, 56.93) and DIPEA (14.48 g, 113.79 mmol) in DMF (150 mL) was stirred at room temperature for 12 hours. After the reaction was completed, the reaction was diluted with water (200 mL), extracted with EtOAc (200 mL×3). The combined organic layers were washed with brine, dried over Na₂SO₄, concentrated in vacuo to give the crude, which was purified with silica gel chromatography (petroleum ether/ethyl acetate, 2:1) to provide RAGE 407-3 (7.3 g, yield: 65.9%) as a yellow solid. LC-MS [M+H]⁺: 293.1.

### Example 21C: Preparation of Compound RAGE 407-4

To a solution of (7-chloro-2-hydroxyquinolin-4-yl)(morpholino)methanone (4 g, 13.65 mmol) and TEA (4.14 g, 40.95 mmol) in DCM (65 mmol) was added trifluoromethanesulfonic anhydride (6.93 g, 24.57 mmol) at 0° C. The reaction was stirred at 0° C. for another 2 hours. After the reaction was completed, the reaction was quenched with water (65 mL), extracted with DCM (60 mL×3). The combined organic layers were washed with brine (100 mL), dried over Na2SO4, concentrated in vacuo to give the crude, which was purified by silica gel column chromatography (petroleum ether/ethyl acetate, 3:1) to provide 407-4 (3.9 g, yield: 67.36%) as a yellow solid. LC-MS [M+H]⁺: 425.1.

### Example 21D: Preparation of Compound RAGE 407-5

A mixture of 7-chloro-4-(morpholine-4-carbonyl)quinolin-2-yl trifluoromethanesulfonate (580 mg, 1.37 mmol), tert-butyl 2-(4-(4,4,5,5-tetramethyl-1,3,2-dioxaborolan-2-yl)phenyl)pyrrolidine-1-carboxylate (765.4 mg, 2.05 mmol), Pd(dppf)Cl₂ (100 mg, 0.137 mmol), K3PO4 (871.3 mg, 4.11 mmol) in 1,4-dioxane/H2O (20 mL/2 mL) was stirred at 80° C. for 12 hours under N₂ atmosphere. After the reaction was completed, the reaction diluted with water (30 mL), extracted with EtOAc (30 mL×3). The combined organic layers were washed with brine (40 mL), dried over Na₂SO₄, concentrated in vacuo to give the crude, which was purified by silica gel column chromatography (petroleum ether/ethyl acetate, 1:1) to provide RAGE 407-5 (550 mg, yield: 76.92%) as a yellow solid. LC-MS [M+H]⁺: 522.2.

### Example 21E: Preparation of Compound RAGE 407-6

A mixture of tert-butyl 2-(4-(7-chloro-4-(morpholine-4-carbonyl)quinolin-2-yl)phenyl)pyrrolidine-1-carboxylate (550 mg, 1.05 mmol), Zn(CN)₂ (246.9 mg, 2.11 mmol), X-phos (100 mg, 0.21 mmol) and [Pd(Allyl)Cl]₂ (40.3 mg, 0.11 mmol) in DMF (5 mL) was degassed by N₂ for 10 min, then sealed and heated with microwave at 130° C. for 1 hour. After the reaction was completed, the reaction was cooled to room temperature, diluted with H₂O (30 mL), extracted with EtOAc (30 mL×3). The combined organic layers were washed with brine (40 mL), dried over Na₂SO₄, concentrated in vacuo to give the crude, which was purified with silica gel chromatography (petroleum ether/ethyl acetated, 1/1) to provide RAGE 407-6 (510 mg, yield: 94.8%) as a yellow solid. LC-MS [M+H]⁺: 513.3.

### Example 21F: Preparation of Compound RAGE 407

To a solution of tert-butyl 2-(4-(7-cyano-4-(morpholine-4-carbonyl)quinolin-2-yl)phenyl)pyrrolidine-1-carboxylate (100 mg, 0.19 mmol) in 1,4-dioxane (4 mL) was added HCl (0.5 mL, 4 M solution in 1,4-dioxane, 2 mmol). The resulting reaction was stirred at room temperature for 3 hours. After the reaction was completed, the reaction was concentrated in vacuo to give the crude, which was purified with Prep-HPLC to provide RAGE 407 (20 mg, yield: 25.5%) as a white solid. LC-MS [M+H]⁺: 413.2, Rt=0.988 min. Purity: 97.60 (214 nm), 97.66 (254 nm). ¹H NMR (400 MHz, DMSO-d6) δ 8.68 (s, 1H), 8.67 (s, 1H), 8.28 (d, J=8 Hz, 2H), 7.93-7.92 (m, 1H), 7.78-7.73 (m, 1H), 7.58 (d, J=8 Hz, 2H), 4.20-4.18 (m, 1H), 4.16-4.03 (m, 4H), 3.78-3.76 (m, 1H), 3.52-3.50 (m, 1H), 3.07-2.97 (m, 3H), 2.93-2.90 (m, 1H), 2.21-2.16 (m, 1H), 1.86-1.56 (m, 2H), 1.23-1.22 (m, 1H).

### Example 22: Synthesis of Analog RAGE 408 According to the Invention

Compound RAGE 408 according to the present disclosure was prepared as shown in Scheme 22 and described below.

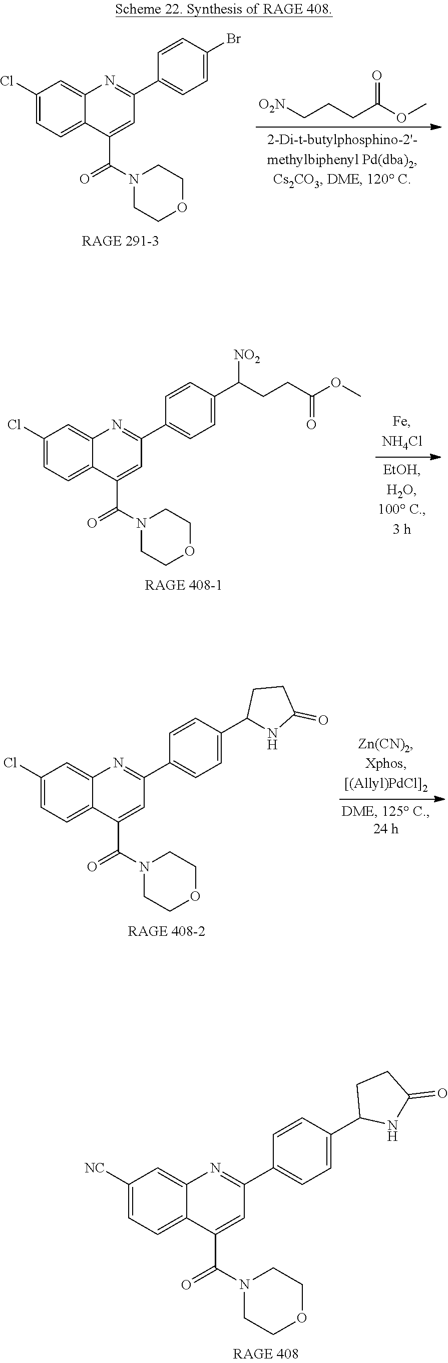

### Example 22A: Preparation of Compound RAGE 408-1

A mixture of (2-(4-bromophenyl)-7-chloroquinolin-4-yl)(morpholino)methanone (2.06 g, 4.8 mmol), methyl 4-nitrobutanoate (1.41 g, 9.6 mmol), 2-Di-t-butylphosphino-2′-methylbiphenyl (299.5 mg, 0.96 mmol), Pd₂(dba)₃ (439 mg, 0.48 mmol) and Cs₂CO₃ (3.91 g, 12 mmol) in DME (50 mL) was stirred at 120° C. for 12 hours. After the reaction was completed, the reaction was diluted with water (50 mL), extracted with EtOAc (60 mL×3). The combine organic layers were washed with brine (60 mL), dried over Na₂SO₄, concentrated in vacuo to give the crude, which was purified by silica gel column chromatography (DCM/MeOH, 15:1) to provide RAGE 408-1 (800 mg, yield: 33.5%) as a yellow solid. LC-MS [M+H]⁺: 498.2.

### Example 22B: Preparation of Compound RAGE 408-2

A mixture of methyl 4-(4-(7-chloro-4-(morpholine-4-carbonyl)quinolin-2-yl)phenyl)-4-nitrobutanoate (800 mg, 1.61 mmol), Fe (924 mg, 16.5 mmol) and NH₄Cl (1.75 g, 33 mmol) in EtOH/water (5 mL/5 mL) was stirred at 100° C. for 3 hours. After the reaction was completed, the reaction mixture was filtered, washed with MeOH (30 mL). The filtrate was concentrated in vacuo to give the crude, which was purified by silica gel column chromatography (DCM/MeOH, 10:1) to provide RAGE 408-2 (210 mg, yield: 30%) as a yellow solid. LC-MS [M+H]⁺: 436.2.

### Example 22C: Preparation of Compound RAGE 408

A mixture of 5-(4-(7-chloro-4-(morpholine-4-carbonyl)quinolin-2-yl)phenyl) pyrrolidin-2-one (174.4 mg, 0.4 mmol), Zn(CN)₂ (93.6 mg, 0.8 mmol), Xphos (38.1 mg, 0.08 mmol) and [Pd(Allyl)Cl]₂ (14.6 mg, 0.04 mmol) in DME (5 mL) was stirred at 125° C. for 24 hours. After the reaction was completed, the reaction was cooled to room temperature, diluted with H₂O (20 mL), extracted with EtOAc (30 mL×3). The combined organic layers were washed with brine (40 mL), dried over Na₂SO₄, concentrated in vacuo to give the crude, which was purified by Prep-HPCL to provide RAGE 408 (35 mg, yield: 20.6%) as a white solid. LC-MS [M+H]⁺: 427.2, Rt=1.138 min. Purity: 99.56 (214 nm), 99.41 (254 nm). ¹H NMR (400 MHz, DMSO-d6) δ8.69 (s, 1H), 8.36-8.34 (m, 2H), 8.19 (s, 1H), 8.02-8.01 (m, 1H), 7.95-7.93 (m, 1H), 7.52 (d, J=8 Hz, 2H), 4.80-4.77 (m, 1H), 3.89-3.76 (m, 1H), 3.72-3.68 (m, 3H), 3.64-3.62 (m, 1H), 3.40-3.41 (m, 1H), 3.27-3.24 (m, 1H), 3.23-3.20 (m, 1H), 3.18-3.15 (m, 1H), 2.51-2.50 (m, 1H), 2.31-2.27 (m, 2H), 1.80-1.76 (m, 1H).

### Example 23: Synthesis of Analog RAGE 409 According to the Invention

Compound RAGE 409 according to the present disclosure was prepared as shown in Scheme 23 and described below.

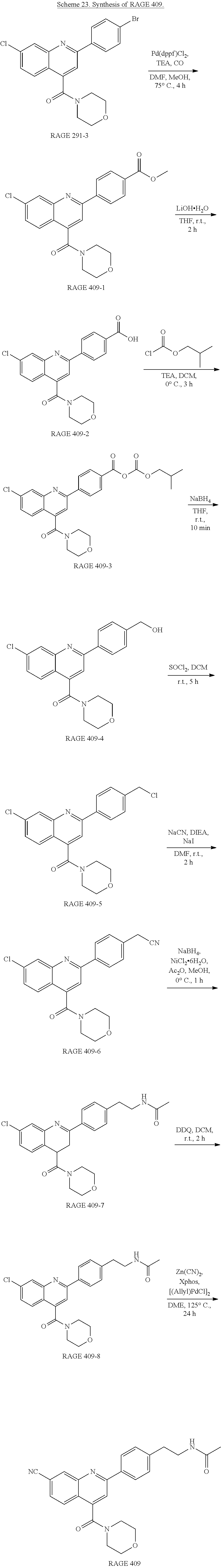

### Example 23A: Preparation of Compound RAGE 409-1

A mixture of (2-(4-bromophenyl)-7-chloroquinolin-4-yl)(morpholino)methanone (5 g, 11.6 mmol), Pd(dppf)Cl₂ (841 mg, 1.16 mmol) and TEA (5.86 g, 58 mmol) in DMF/MeOH (60 mL/60 mL) was stirred at 75° C. for 4 hours under CO atmosphere. After the reaction was completed, the reaction was diluted with water (150 mL), extracted with EtOAc (100 mL×3). The combined organic layers were washed with brine (80 mL), dried over Na₂SO₄, concentrated in vacuo to give the crude product which was purified by silica gel column chromatography (petroleum ether/ethyl acetate, 3:1) to provide RAGE 409-1 (3.2, yield: 67.28%) as a white solid. LC-MS [M+H]⁺: 411.2.

### Example 23B: Preparation of Compound RAGE 409-2

A mixture of methyl 4-(7-chloro-4-(morpholine-4-carbonyl)quinolin-2-yl)benzoate (3.2 g, 7.8 mmol) and LiOH—H₂O (0.49 g, 11.7 mmol) in THF/H₂O (60 mL/15 mL) was stirred at room temperature for 2 hours. After the reaction was completed, the reaction was concentrated in vacuo to remove THE, and the diluted was diluted with water (30 mL). The pH of aqueous layer was adjusted to 3 by HCl (1N), and the white solid was formed. The mixture was filtered, and washed with water (100 mL). The filter cake was dried in vacuo to provide the compound RAGE 409-2 (2.3 g, yield: 74.2%) as a white solid. LC-MS [M+H]⁺: 397.2.

### Example 23C: Preparation of Compound RAGE 409-3

To a solution of 4-(7-chloro-4-(morpholine-4-carbonyl)quinolin-2-yl)benzoic acid (1.5 g, 3.78 mmol) and TEA (763.6 mg, 7.56 mmol) in DCM (50 mL) was added isobutyl chloroformate (668.3 mg, 4.9 mmol) slowly at 0° C. The resulting reaction was stirred at 0° C. for 3 hours. After the reaction was completed, the reaction was quenched with water (50 mL), extracted with DCM (60 mL×3). The combined organic layers were washed with brine (60 mL), dried over Na₂SO₄, concentrated in vacuo to give the crude product which was purified by silica gel column chromatography (petroleum ether/ethyl acetate, 4:1) to provide RAGE 409-3 (1.45 g, yield: 77.1%) as a white solid. LC-MS [M+H]⁺: 497.1.

### Example 23D: Preparation of Compound RAGE 409-4

To a solution of 4-(7-chloro-4-(morpholine-4-carbonyl)quinolin-2-yl)benzoic (isobutyl carbonic) anhydride (1 g, 2.01 mmol) in THF (25 mL) was added NaBH₄ (152 mg, 4 mmol) at 0° C. The resulting mixture was stirred at room temperature for 10 min. After the reaction was completed, the reaction was quenched with water (30 mL), extracted with EtOAc (40 mL×3). The combined organic layers were washed with brine (40 mL), dried over Na₂SO₄, concentrated in vacuo to give the crude product which was purified by silica gel column chromatography (petroleum ether/ethyl acetate, 1:1) to provide RAGE 409-4 (650 mg, yield: 84.4%) as a white solid. LC-MS [M+H]⁻: 383.2.

### Example 23E: Preparation of Compound RAGE 409-5

To a solution of (7-chloro-2-(4-(hydroxymethyl)phenyl)quinolin-4-yl)(morpholino)methanone (650 mg, 1.7 mmol) in DCM (20 mL) was added SOCl₂ (2 mL). The resulting reaction was stirred at room temperature for 5 hours. After the reaction was completed, the reaction was concentrated in vacuo give the RAGE 409-5 (650 mg, crude) as a yellow solid. LC-MS [M+H]⁺: 401.2.

### Example 23F: Preparation of Compound RAGE 409-6

A mixture of (7-chloro-2-(4-(chloromethyl)phenyl)quinolin-4-yl) (morpholino)methanone (650 mg, crude), DIPEA (890.1 mg, 6.9 mmol), NaI (20.7 mg, 0.14 mmol) and NaCN (135 mg, 2.76 mmol) in DMF (20 mL) was stirred at room temperature for 2 hours. After the reaction was completed, the reaction was quenched with water (30 mL), extracted with EtOAc (40 mL×3). The combined organic layers were washed with brine (40 mL), dried over Na₂SO₄, concentrated in vacuo to give the crude product which was purified by silica gel column chromatography (petroleum ether/ethyl acetate, 3:1) to provide RAGE 409-6 (485 mg, yield: 73% over 2 steps) as a yellow solid. LC-MS [M+H]⁺: 392.2.

### Example 23G: Preparation of Compound RAGE 409-7

To a solution of 2-(4-(7-chloro-4-(morpholine-4-carbonyl)quinolin-2-yl)phenyl) acetonitrile (485 mg, 1.24 mmol) in MeOH (20 mL) was added acetic anhydride (316.2 mg, 3.1 mmol), NiCl2-6H2O (293.9 mg, 1.24 mmol), followed by NaBH₄ (70.7 mg, 1.86 mmol) at 0° C. The resulting was stirred at 0° C. for 1 hour. After the reaction was completed, the reaction was quenched with water (25 mL), extracted with EtOAc (30 mL×3). The combined organic layers were washed with brine (40 mL), dried over Na₂SO₄, concentrated in vacuo to give the crude product which was purified by silica gel column chromatography (petroleum ether/ethyl acetate, 3:1) to provide RAGE 409-7 (472 mg, yield: 86.5%) as a white solid. LC-MS [M+H]⁺: 440.1.

### Example 23H: Preparation of Compound RAGE 409-8

To a solution of N-(4-(7-chloro-4-(morpholine-4-carbonyl)-3,4-dihydroquinolin-2-yl) phenethyl) acetamide (472 mg, 1.07 mmol) in DCM (10 mL) was added 1,2-Dichloro-4,5-Dicyanobenzoquinone (487 mg, 2.14 mmol). The reaction was stirred at room temperature for 2 hours. After the reaction was completed, the reaction was quenched with water (25 mL), extracted with DCM (30 mL×3). The combined organic layers were washed with brine (40 mL), dried over Na₂SO₄, concentrated in vacuo to give the crude product which was purified by silica gel column chromatography (petroleum ether/ethyl acetate, 2:1) to provide RAGE 409-8 (245 mg, yield: 52.3%) as a yellow solid. LC-MS [M+H]⁺: 438.3.

### Example 23I: Preparation of Compound RAGE 409

N-(4-(7-chloro-4-(morpholine-4-carbonyl)quinolin-2-yl) phenethyl) acetamide (175.2 mg, 0.4 mmol), Zn(CN)₂ (93.6 mg, 0.8 mmol), Xphos (38.1 mg, 0.08 mmol) and [Pd(Allyl)Cl]₂ (14.6 mg, 0.04 mmol) in DME (5 mL) was stirred at 125° C. for 24 hours. After the reaction was completed, the reaction was cooled to room temperature, diluted with H₂O (30 mL), extracted with EtOAc (30 mL×3). The combined organic layers were washed with brine (40 mL), dried over Na₂SO₄, concentrated in vacuo to give the crude, which was purified by Prep-HPCL to provide RAGE 409 (35 mg, yield: 20.5%) as a white solid. LC-MS [M+H]⁺: 429.2, Rt=1.042 min. Purity: 96.74 (214 nm), 96.83 (254 nm). ¹H NMR (400 MHz, DMSO-d6) δ 8.67 (s, 1H), 8.34 (s, 1H), 8.28 (d, J=8.9 Hz, 2H), 8.01-7.91 (m, 3H), 7.43 (d, J=8.9 Hz, 2H), 3.89-3.77 (m, 6H), 3.33-3.30 (m, 2H), 3.24-3.14 (m, 2H), 2.82-2.78 (m, 2H), 1.80 (s, 3H).

### Example 24: Synthesis of Analog RAGE 410 According to the Invention

Compound RAGE 410 according to the present disclosure was prepared as shown in Scheme 24 and described below.

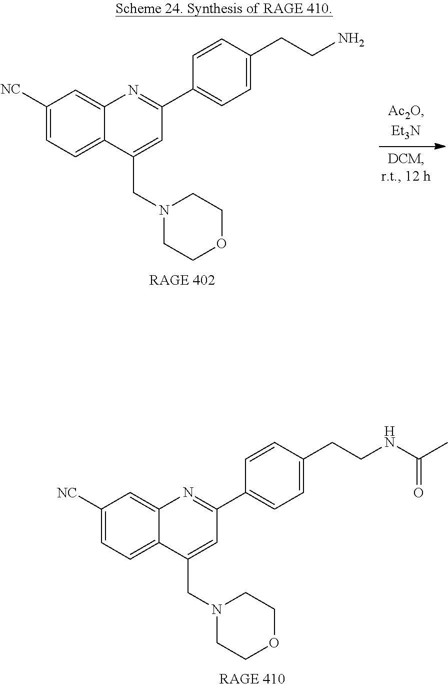 [SMILES: N#Cc1ccc2c(CN3CCOCC3)cc(-c3ccc(CCN)cc3)nc2c1 ; CC(=O)NCCc1ccc(-c2cc(CN3CCOCC3)c3ccc(C#N)cc3n2)cc1]

### Example 24A: Preparation of Compound RAGE 410

To a solution of 2-(4-(2-aminoethyl)phenyl)-4-(morpholinomethyl)quinoline-7-carbonitrile (40 mg, 0.11 mmol) in dry DCM (2 mL) was added TEA (33 mg, 0.33 mmol), followed by Ac₂O (17 mg, 0.17 mmol). The resulting mixture was stirred at room temperature for 12 h. The reaction was diluted with H₂O (20 mL), extracted with DCM (30 mL×3). The combined organic layers were washed with brine (20 mL), dried over Na₂SO₄, concentrated in vacuo to give the crude product, which was purified by Prep-TLC (DCM:MeOH=20:1) to provide the desired RAGE 410 (10.3 mg, yield: 22.6%) as a white solid. LC-MS [M+H]⁺: 415.2, Rt=0.982 min. Purity: 97.55 (214 nm), 97.37 (254 nm). ¹H NMR (400 MHz, DMSO-d6) δ 8.55 (s, 1H), 8.47 (d, J=4 Hz, 1H), 8.34-8.21 (m, 3H), 7.87 (s, 1H), 7.75 (t, J=4 Hz, 1H), 7.42 (d, J=8 Hz, 2H), 4.00 (s, 2H), 3.58 (s, 4H), 3.38-3.33 (m, 2H), 2.82-2.76 (m, 2H), 2.52-2.49 (m, 4H), 1.82 (s, 3H).

### Example 25: Delayed Type Hypersensitivity in Animals Treated with RAGE 229, RAGE 406R, RAGE 406S by Oral Gavage

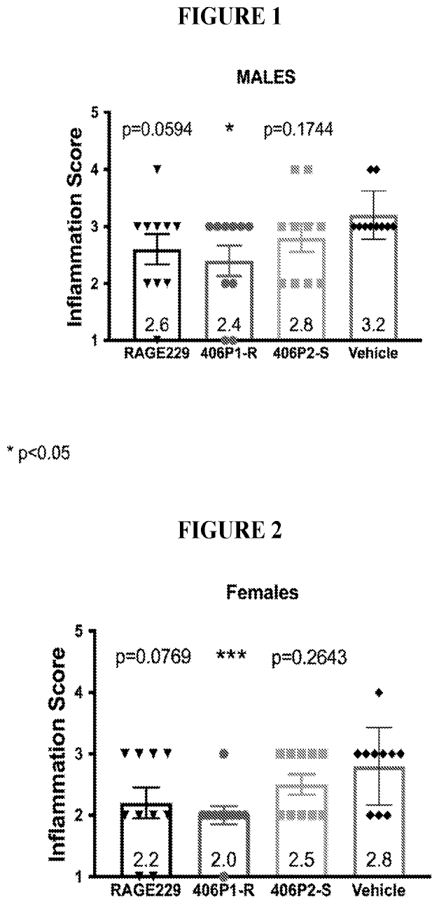

Male and female CF-1 mice were sensitized over the left inguinal lymph node with a methylated bovine serum albumin emulsion (mBSA, NaCl, dextran and Freund's incomplete adjuvant). On day 19 and 20 after sensitization, mice received oral gavage dose of the test compounds RAGE203, RAGE208 and RAGE229 (5 mg/kg body weight) or equal volumes of PBS-containing vehicle twice daily for a total of four doses, last dose given 12 hours before the final read out of the immune response. After the final compound delivery, mBSA was injected into the left plantar hind paw. The following morning, the injected paw was scored by investigators naïve to the experimental condition using a semiquantitative scoring system. Results are shown in FIGS. 1 and 2.

### Example 26: Fluorescence Binding Titration Assay

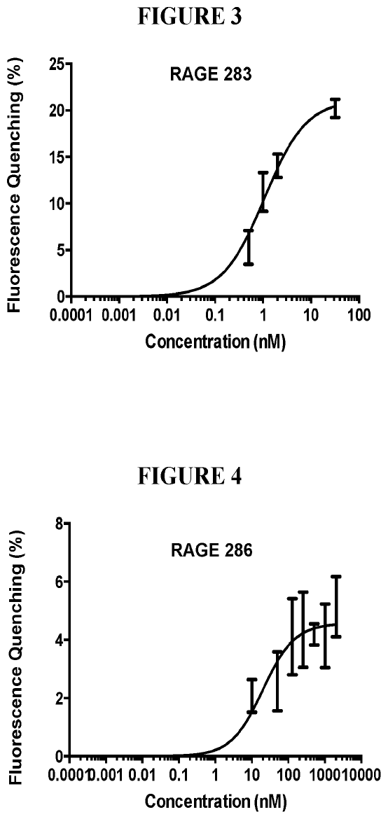

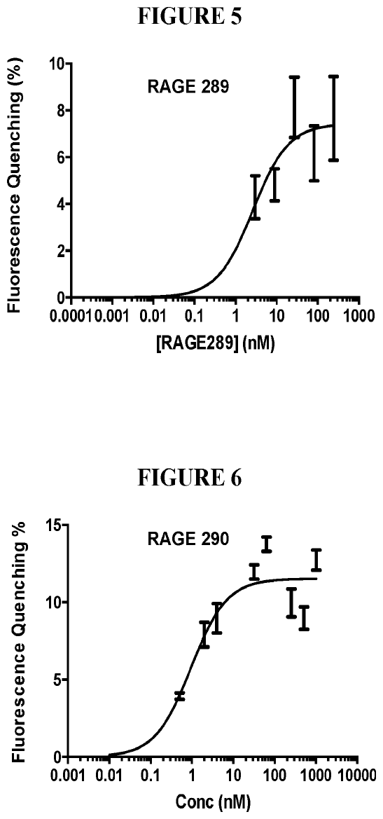

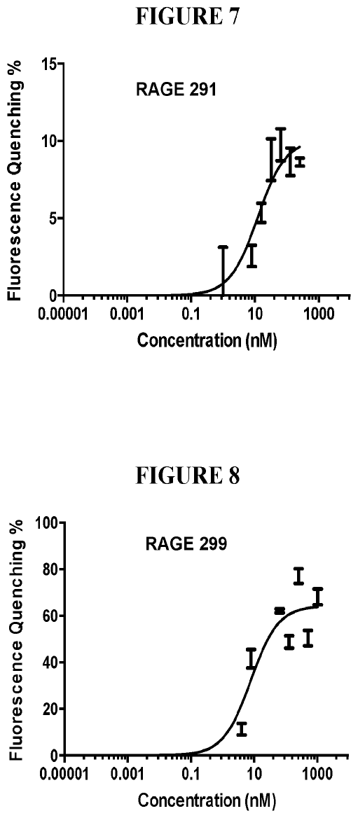

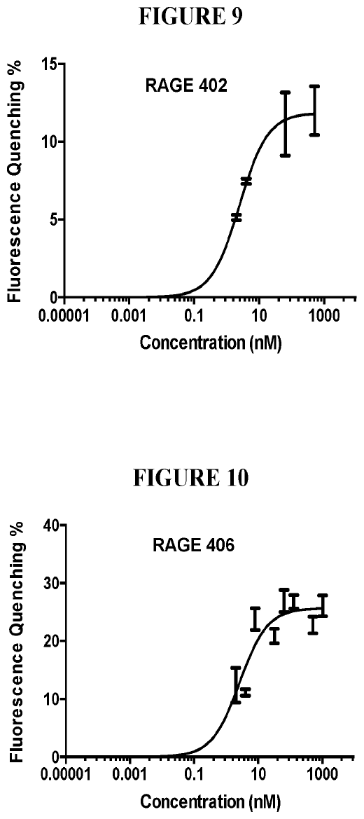

Native tryptophan fluorescence experiments were conducted using a Horiba Jobin Yvon Fluorolog spectrofluorometer. 1 mM of a compound was dissolved in 10 mM phosphate buffer [pH 7.0] and 50% DMSO. 20 nM ctRAGE solution was individually titrated from 0.1 nM-100 μM with the compounds in 100 μL of 10 mM phosphate buffer [pH 7.0] and 5% DMSO. The excitation and emission wavelengths were 280 nm and 352 nm, respectively. Dissociation constants, K_{d}, were estimated from the changes in peak fluorescence intensities as a function of the free compound concentration by using Prism 6 software (GraphPad). Data were fit to the equation, (F−F₀)/Fₘₐₓ=[compound]/(K_{d}+[compound]) where F is the fluorescence intensity at a given compound concentration, F₀ is the fluorescence intensity of the blank, and Fₘₐₓ is the maximum fluorescence intensity. Results are shown in FIGS. 3-11 and K_{D} values are presented in Table 1 below.

TABLE 1
Results of Fluorescence binding titration assay.

| Compound | |
|---|---|
| No. | K_{D} (nM) |
| RAGE 283 | 1 |
| RAGE 286 | 20 |
| RAGE 289 | 3 |
| RAGE 290 | 1 |
| RAGE 291 | 12 |
| RAGE 299 | 8 |
| RAGE 402 | 3 |
| RAGE 406 | 3 |
| RAGE 406R | 0.6 |
| RAGE 406S | 2 |
| RAGE 407 | 2 |

### Example 27: Murine Primary Aortic Smooth Muscle Cell (SMC) Migration

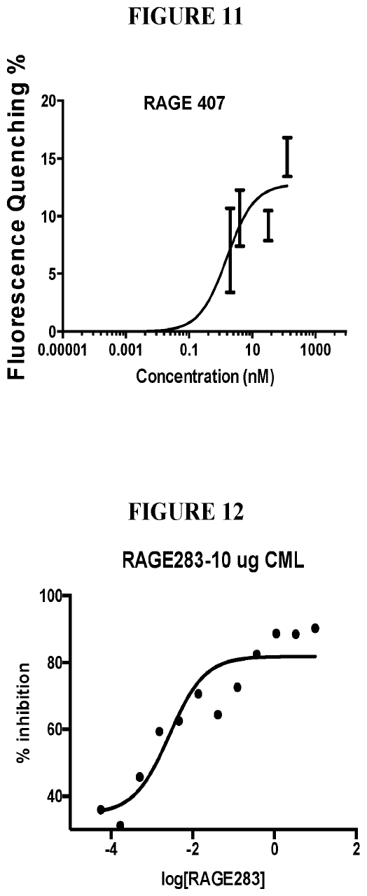

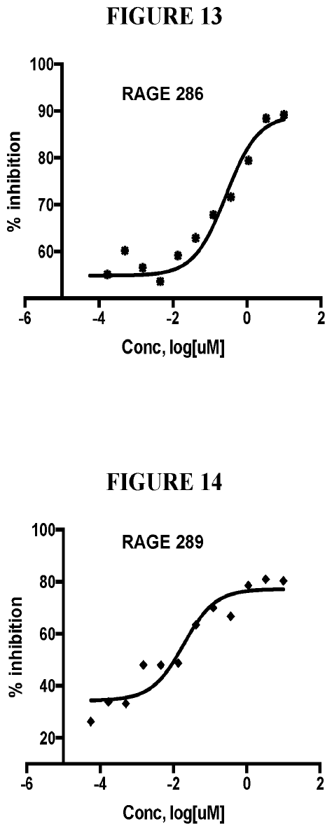

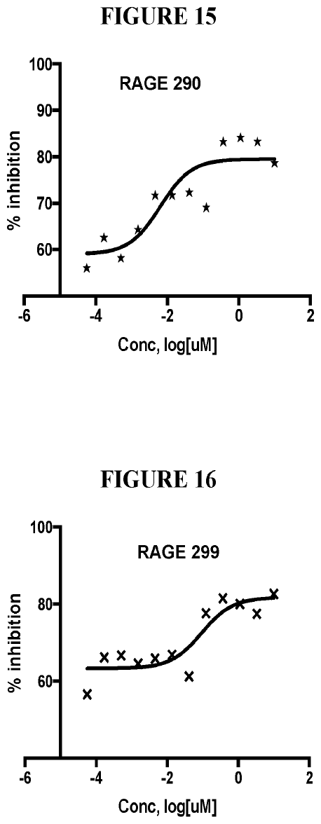

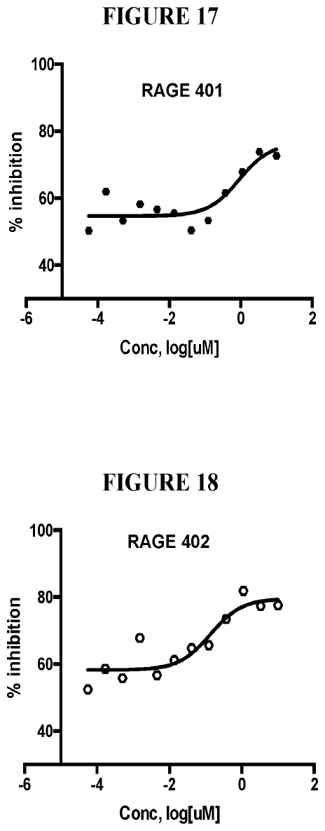

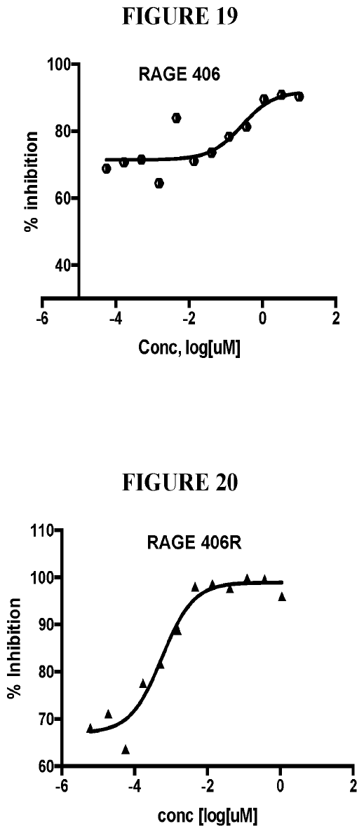

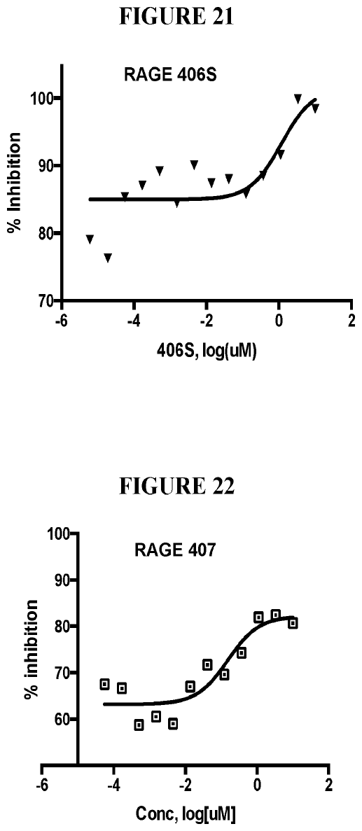

Murine SMCs were cultured from the aortas of 10-week old male mice as described in M. B. Manigrasso, J. Pan, V. Rai, J. Zhang, S. Reverdatto, N. Quadri, R. J. DeVita, R. Ramasamy, A. Shekhtman, A. M. Schmidt, Small Molecule Inhibition of Ligand-Stimulated RAGE-DIAPH1 Signal Transduction. Scientific reports 6, 22450 (2016). Wild-type murine aortic SMCs were isolated and used between passages 8 to 12. Migration in response to the RAGE ligand CML-AGE (CML, 10 μg/ml) or a general effector, not a RAGE ligand, PDGF-BB, 10 ng/ml (R&D systems) was assessed with a wounding assay as also described in in M. B. Manigrasso, J. Pan, V. Rai, J. Zhang, S. Reverdatto, N. Quadri, R. J. DeVita, R. Ramasamy, A. Shekhtman, A. M. Schmidt, Small Molecule Inhibition of Ligand-Stimulated RAGE-DIAPH1 Signal Transduction. Scientific reports 6, 22450 (2016). Briefly, cells were grown to confluence in 24-well plates and starved overnight. The following morning, serum free media (SFM) was removed and compounds were added per dose response. Immediately following the addition of compounds, the monolayer was wounded using a p10 pipette tip. The compounds were allowed to incubate for 1.5 h. Following this incubation, all compounds were removed and fresh media containing RAGE ligand, CML-AGE (10 μg/ml), or general effector, PDGF-BB (10 ng/ml), was added for 4 h. Cells were maintained at 37° C. and 5% CO2. Images were taken at 4 h. Each image was measured, and an area ingrowth of effective migrating cells was calculated. Results are shown in FIGS. 12-20 and Table 2 below.

TABLE 2
Results of Murine SMC Migration Assay.

| Compound No. | IC₅₀ (nM) |
|---|---|
| RAGE 283 | 2.6 |
| RAGE 286 | 270 |
| RAGE 289 | 18 |
| RAGE 290 | 6 |
| RAGE 299 | 93 |
| RAGE 401 | 905 |
| RAGE 402 | 136 |
| RAGE 406 | 28 |
| RAGE 406R | 1 |
| RAGE 406S | 1162 |
| RAGE 407 | 153 |

### Example 28: Wound Healing

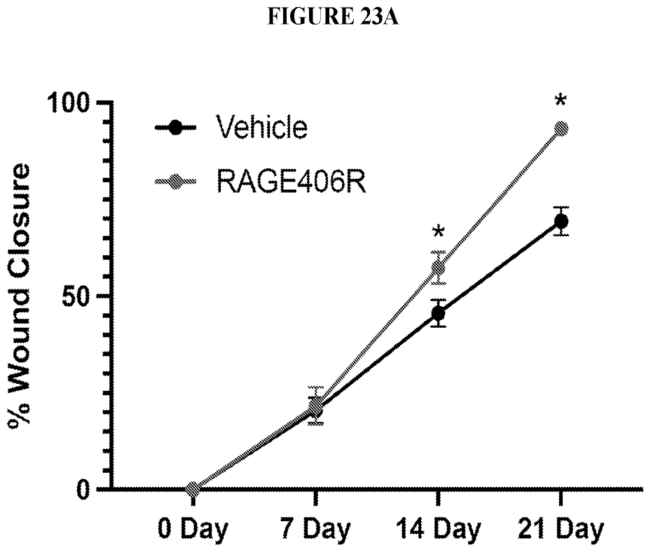

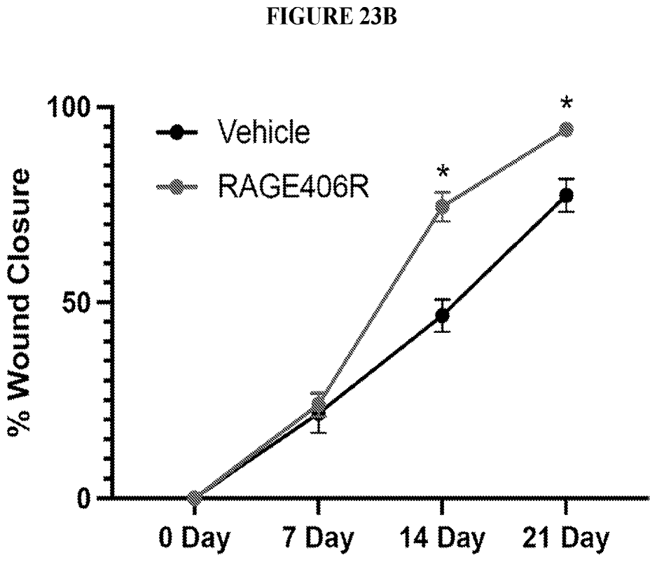

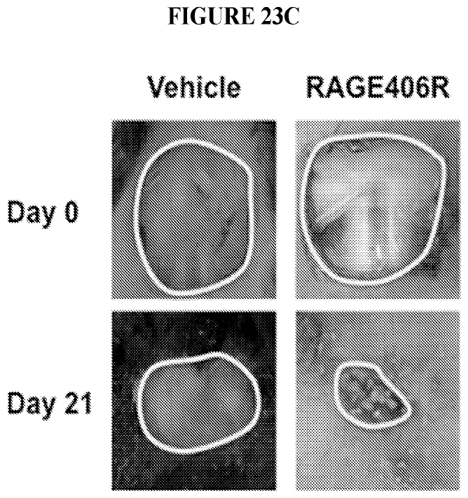

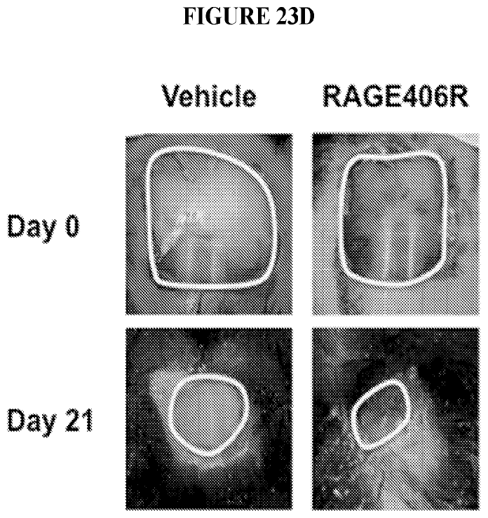

RAGE 406R was tested in male and female ob ob mice (model of obesity and type 2 diabetes) for potential benefits in wound healing. Full thickness excisional wounds were created on the backs of 8 week old ob ob mice, as described in Manigrasso et al., “Small-molecule antagonism of the interaction of the RAGE cytoplasmic domain with DIAPH1 reduces diabetic complications in mice,” Sci Transl Med. 13 (621):eabf7084 (2021). Beginning on day 3 after the surgery, mice received RAGE 406R (5 mg/kg) twice daily by topical administration under the Tegaderm through day 10 post-operatively. The wounds were photographed on days 0, 7, 14, and 21 and the % wound closure (compared to baseline) was calculated for each mouse. The Mean+/−Standard Error of the Mean is shown in FIGS. 23A-B along with representative images (FIGS. 23C-D).

TABLE 3
The Numbers of Mice Tested

| | Vehicle | RAGE406r |
|---|---|---|
| MALE | 10 | 8 |
| FEMALE | 7 | 10 |

In both male and female mice, on days 14 and 21 post-surgery, mice treated with RAGE406r demonstrated significantly higher wound closure compared to vehicle-treated animals. Bar graphs and representative images of the male/female mice wounds on days 0 and 21 are shown in FIGS. 23A-D (the reflection of the open/unhealed wound in FIGS. 23 C-D is circled).

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

1-47. (canceled)

48. A compound having a structure of Formula (I): 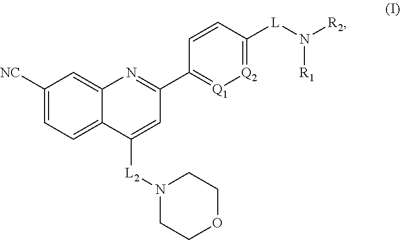
wherein:
Q₁ and Q₂ are independently ═CH— or ═N—, with a proviso that Q₁ and Q₂ are not both ═N—;
L₂ is —C(═O)—, —C(═NH)—, or a C₁-C₆ alkylene;
R₁ and L₁ are fused to make a 5- or 6-membered optionally substituted heterocycle;
R₂ is hydrogen, a C₁-C₆ alkyl, or R₁ and R₂ are fused to make a 5- or 6-membered optionally substituted heterocycle,
or a pharmaceutically acceptable salt thereof.

49. The compound of claim 48, wherein R₁ and L₁ are fused to make

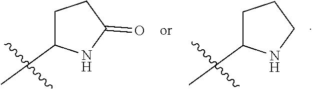 [SMILES: *C1CCC(=O)N1 ; *C1CCCN1]

50. The compound of claim 48, wherein Q₁ and Q₂ are both ═CH—.

51. The compound of claim 48, wherein L₂ is a C₁-C₆ alkylene, optionally wherein L₂ is a —CH₂—.

52. The compound of claim 48, wherein R₂ is hydrogen or is a C₁-C₆ alkyl.

53. The compound of claim 52, wherein R₂ is —CH₃.

54. The compound of claim 48, wherein the compound is selected from the group consisting of: 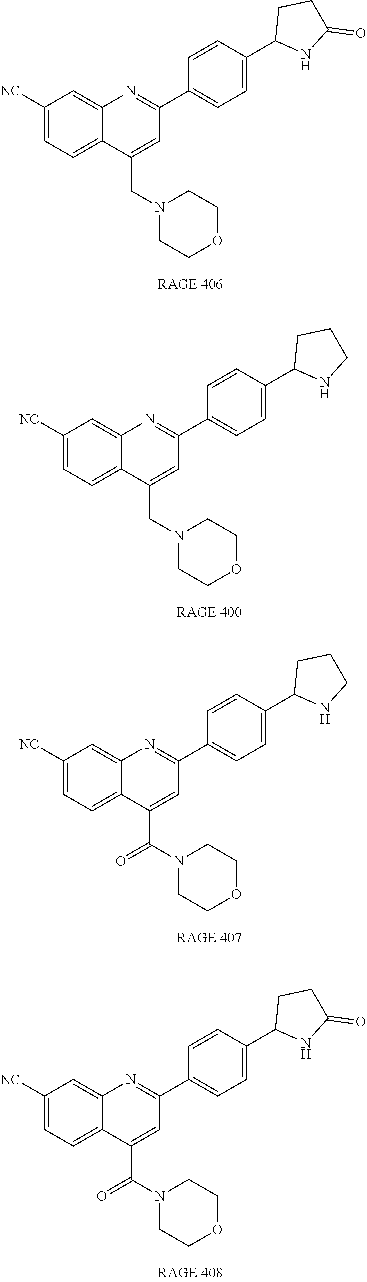 [SMILES: N#Cc1ccc2c(CN3CCOCC3)cc(-c3ccc(C4CCC(=O)N4)cc3)nc2c1 ; N#Cc1ccc2c(CN3CCOCC3)cc(-c3ccc(C4CCCN4)cc3)nc2c1 ; N#Cc1ccc2c(C(=O)N3CCOCC3)cc(-c3ccc(C4CCCN4)cc3)nc2c1 ; N#Cc1ccc2c(C(=O)N3CCOCC3)cc(-c3ccc(C4CCC(=O)N4)cc3)nc2c1]
or a pharmaceutically acceptable salt thereof.

55. The compound of claim 48, comprised of: 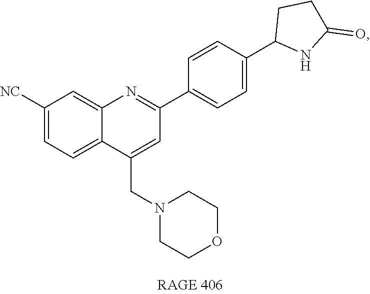 [SMILES: N#Cc1ccc2c(CN3CCOCC3)cc(-c3ccc(C4CCC(=O)N4)cc3)nc2c1]
or a pharmaceutically acceptable salt thereof.

56. The compound of claim 48, comprised of: 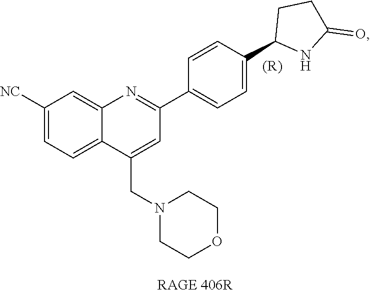
or a pharmaceutically acceptable salt thereof.

57. A pharmaceutical composition comprising the compound of claim 48 or a pharmaceutically acceptable salt thereof and a pharmaceutically acceptable carrier.

58. A pharmaceutical dosage form comprising the compound of claim 48 or a pharmaceutically acceptable salt thereof.

59. A method of modulating the activity of the receptor for advanced glycation end products (RAGE) in a cell, comprising contacting the cell with an effective amount of the compound of claim 48 or the pharmaceutically acceptable salt thereof.

60. The method of claim 59, wherein the modulating the activity of RAGE comprises inhibiting the activity of RAGE.

61. A method of treating or ameliorating a disease or condition associated with RAGE activity in a subject in need thereof, comprising administering to the subject a therapeutically effective amount of the compound of claim 48 or the pharmaceutically acceptable salt thereof.

62. The method according to claim 61, wherein the disease or condition is selected from diabetes and its complications, insulin resistance, atherosclerosis, peripheral vascular disease and associated complications, cardiovascular disease nephropathy, retinopathy, cardio- and cerebrovascular ischemia/reperfusion injury, heart attack, myocardial infarction, ischemic cardiomyopathy, cancer, tumor invasion and metastases, acute and chronic inflammation, autoimmune diseases, neurodegeneration, arthritis, allergy asthma, obesity, pollution-associated tissue or organ damage, infection and its complications, sepsis, pneumonia, liver injury/damage, amyloidoses, skin disorders, colitis, lupus, and impaired wound healing, optionally wherein the disease or condition is diabetes and its complications; or wherein the disease or condition is COVID-19 infection and its complications.

63. A method of treating or ameliorating diabetes and its complications in a subject in need thereof, comprising administering to the subject a therapeutically effective amount of the compound of claim 48 or the pharmaceutically acceptable salt thereof.

64. The method of claim 63, wherein the subject is a mammal.

65. The method according to claim 59, wherein the compound is administered orally, rectally, intraocularly, transdermally, subcutaneously, intravenously, intramuscularly, intraperitoneally, interdermally, directly into cerebrospinal fluid, intratracheally, or intranasally.

66. The method according to claim 61, wherein the compound is administered orally, rectally, intraocularly, transdermally, subcutaneously, intravenously, intramuscularly, intraperitoneally, interdermally, directly into cerebrospinal fluid, intratracheally, or intranasally.
